(19) United States
(12) Patent Application Publication
(10) Pub. No.: US 2024/0350481 A1
(43) Pub. Date: Oct. 24, 2024

(54) USE OF N-MYRISTOYL TRANSFERASE (NMT) INHIBITORS IN THE TREATMENT OF CANCER, AUTOIMMUNE DISORDERS, AND INFLAMMATORY DISORDERS

(71) Applicant: PACYLEX PHARMACEUTICALS INC., Calgary (CA)

(72) Inventors: Luc G. BERTHIAUME, Edmonton (CA); Erwan BEAUCHAMP, Edmonton (CA)

(21) Appl. No.: 18/031,772

(22) PCT Filed: Oct. 20, 2021
(86) PCT No.: PCT/CA2021/051475

§ 371 Date: Apr. 13, 2023

Related U.S. Application Data
(60) Provisional application No. 63/093,970, filed on Oct. 20, 2020.

(51) Int. Cl. A61K 31/496; A61K 31/416; A61P 37/02

(52) U.S. Cl. CPC A61K 31/496; A61K 31/416; A61P 37/02

(57) ABSTRACT

The use of N-myristoyl-transferase (NMT) inhibitors in the treatment of cancer, autoimmune disorders, and inflammatory disorders is disclosed. With respect to cancer, the preferred cancer to be treated is B-cell lymphoma, and the NMT used is PCLX-001 (DDD86481, CAS RN 1215011-08-7). Preferred NMT inhibitors for the treatment of autoimmune and inflammatory disorders include the aforementioned PCLX-001, PCLX-002 (DDD85646, CAS RN 1215010-55-10), and IMP-1088 (CAS RN 2059148-82-0), and the disorders to be treated include rheumatoid arthritis, asthma, gastritis, colitis, and other digestive and respiratory ailments.

### CROSS REFERENCE TO RELATED APPLICATION

This application claims priority to U.S. Patent Application No. 63/093,970, filed Oct. 20, 202, the entire contents of which is hereby incorporated by reference.

### FIELD

Targeting N-myristoylation for therapy of B-cell lymphomas, autoimmune disorders, and/or inflammatory disorders.

### BACKGROUND

Hematological cancers such as lymphoma account for approximately 9% of new cancer cases and cancer-related deaths worldwide^{1, 2, 3}. Although patients with aggressive non-Hodgkin lymphomas such as Burkitt lymphoma (BL) and diffuse large B-cell lymphoma (DLBCL) frequently achieve initial remission with current therapies, these are toxic and a substantial proportion of patients experience disease relapse and premature death^{2, 3}. Recent data from the Surveillance, Epidemiology, and End Results (SEER) of the National Cancer Institute (NCI) show a 5-year post diagnosis survival rate for non-Hodgkin lymphoma and DLBCL, relative to age-matched controls, of only 70% and 63%, respectively². The identification of new druggable targets and better-tolerated treatments for aggressive lymphomas are therefore much needed.

While B-cell receptor (BCR) signaling is essential for normal B-cell function, it is often deregulated and provides critical pro-survival signals for B-cell lymphomagenesis in both BL and DLBCL^{4, 5, 6, 7, 8} Indeed, the presence of self-antigens and/or mutations in key BCR effectors impact distinct signaling modes of the BCR. In addition to the ligand activated BCR signaling mode, these include the chronic active BCR signaling in activated B cell-like DLBCL (ABC-DLBCL) and chronic lymphocytic leukemia (CLL) as well as the tonic (antigen independent constitutive baseline signaling) BCR signaling in BLs^{4, 5, 6, 7, 8} Typically, engagement of the BCR leads to the translocation of this receptor to plasma membrane lipid rafts containing the myristoylated Src-family kinase (SFK) Lyn^{9, 10, 11}. Myristoylated Lyn phosphorylates select tyrosine residues in the immune-receptor tyrosine-based motif (ITAM) of the BCR associated CD79A-CD79B heterodimer^{12, 13} resulting in the recruitment of spleen tyrosine kinase (SYK). Human germinal center-associated (HGAL) protein is another myristoylated protein localized to lipid rafts and is phosphorylated upon BCR activation^{14, 15}. Phosphorylated HGAL enhances BCR signaling by augmenting the activation and recruitment of SYK to phosphorylated ITAMs, triggering the tyrosine phosphorylation of the Tec family member Bruton's tyrosine kinase (BTK)¹⁶, phospholipase C_{Υ}, and protein kinase CD (PKCO)¹³. Activated phospholipase C_{Υ} activity produces diacylglycerol (DAG) and inositol-trisphosphate (IP₃), which activate PKCs and mobilize calcium ions from endoplasmic reticulum stores respectively. These chemical mediators, in turn, activate various signaling pathways¹⁷. All these early signaling events promote cell survival and proliferation through activation of transcription via the NFκB, PI3K, extracellular signal regulated kinase (ERK) mitogen-activated protein kinase (MAPK), CREB and NF-AT pathways^{4, 5, 6, 18}. The importance of BCR signaling in lymphomagenesis has prompted the development of numerous pharmacological agents, which target effector proteins downstream of the BCR including various SFKs (dasatinib), BTK (ibrutinib), and PI3Kδ (CAL-101)^{4, 5, 19, 20}.

In humans, protein myristoylation is mediated by two ubiquitously expressed N-myristoyl-transferases, NMT1 and NMT2, which add a 14 carbon fatty acid myristate onto numerous proteins^{21, 22}. Myristoylation plays a fundamental role in cell signaling and allows for the dynamic interactions of proteins with cell membranes^{23, 24} Myristoylation occurs at the N-terminal glycine residue of proteins either co-translationally after the removal of the initiator methionine or post-translationally after caspase-cleavage during apoptosis²³. Up to 600 proteoforms²⁵ in humans are myristoylated and the proper membrane targeting and functions of these proteins require myristoylation^{23, 24, 26, 27, 28}. SFKs, Abl, G_{α} subunits, Arf GTPases, caspase truncated (ct-) Bid and ct-PAK2 are examples of myristoylated proteins that critically regulate cell growth and apoptosis^{23, 29, 30, 31, 32, 33, 34, 35} Recently, NMTs were also shown to be responsible for myristoylation of N-terminally located lysine residues of Arf6 GTPase, thereby adding to their roles in cell signaling^{36, 37}. Because NMTs are essential for the viability of parasites, small molecule inhibitors such as DDD85646 were developed as a T. brucei NMT inhibitor to treat African sleeping sickness³⁸. DDD85646 was also synthesized and validated independently as a bona fide inhibitor of human NMTs under the name IMP-366³⁹. Because NMT expression levels and activity are increased in some cancers^{40, 41, 42, 43, 44, 45}.

Traditionally, autoimmune disorders were classified as T cell mediated or autoantibody mediated. However the improved understanding of the complexity of the immune system has significantly influenced the way we view autoimmune diseases and their pathogeneses. Reciprocal roles of T-cell help for B cells during adaptive immune responses and B-cell help in CD4+ T-cell activation are being increasingly recognized. The observation that most autoantibodies in traditionally autoantibody-mediated diseases are of the IgG isotype and carry somatic mutations strongly suggests T-cell help in the autoimmune B-cell response. Likewise B cells function as crucial antigen presenting cells in autoimmune diseases that are traditionally viewed as T cell mediated. It is thought that most autoimmune diseases are driven by a dysfunction in the immune network consisting of B cells, T cells, and other immune cells.

Signaling through the BCR plays an important role in the generation of antibodies, in autoimmunity, and in the establishment of immunological tolerance.

The role of B cells in the pathogenesis and treatment of rheumatoid arthritis is discussed in Marston, B. et al (2010) Curr Opin Rheumatol. 2616 May; 22(3):307-315. The role of B-cell inhibitors as therapy for rheumatoid Arthritis: An Update, is discussed in Kwan-Morley, J., and Albert, D (2007) Current Rheumatology Reports. 9:461-466. The activation of Syk in peripheral blood B cells in patients with rheumatoid arthritis is discussed in Iwata, S., et al. (2015) Arthritis & rheumatology. Vol 67. No 1. pp 63-73. The role of B cell inhibitory receptors and autoimmunity is discussed in Pritchard, N. R., & Smith, K. G. C. (2003) Immunology. 108. 263-273. The role of B-cell kinase inhibitors in rheumatoid arthritis is discussed in Chu, A. & Chang, BY (2013) OA Arthritis. October 27; 1(2):17. The pathogenic rolls of B cells in human autoimmunity: insights from the clinic, is discussed in Martin, F., and Chan, A. C. (2004) Immunity. Vol 20, 517-527. The ligand recognition determines the role of inhibitory B cell co-receptors in the regulation of B cell homeostasis and autoimmunity, is discussed in Tsubata, T (2018) Frontiers in Immunology. Vol 9. Article 2276. The targeting B cells and plasma cells in autoimmune diseases is discussed in Hofmann, K., et al (2018) Frontiers in Immunology. Vol 9. Article 835. The role of Src Kinase in macrophage-mediated inflammatory responses, is discussed in Byeon, S. E., et al. (2012) Mediators of Inflammation. Volume 2013. 18, pages. R406, an Orally available spleen tyrosine kinase inhibitor block Fc Receptor Signallying and Reduces Immune Complex-Mediated Inflammation, is discusses in Braselmann, S., et al. (2006) JPET. 319:998-1008. The pharmacokinetics of Fostamatinib, a spleen tyrosine kinase (SYK) inhibitor, in healthy human subjects following single and multiple oral dosing in three phase I studies, is described in Bluom, M., et al (2012) Br J Clin Pharmacol. 76:1. 78-88. Regulatory T cells in human disease and their potential for therapeutic manipulation, is discussed in Taams, L. S., et al. (2006) Immunology. 118. 1-9. The role of γδ T cells and inflammatory skin diseases is discussed in Jee, M. H. et al (2020) Immunological Reviews.2020; 00:1-13.

Anti-B Cell receptor (BCR) complex antibodies have therapeutic use in the treatment of autoimmunity, cancer, inflammatory disease, and transplantation.

Inhibiting the T Cell receptor (TCR) signal has promise for treating a broad spectrum of human T cell-mediated autoimmune and inflammatory diseases.

A need remains for an inhibitor of the BCR and/or the TCR, for the use in the treatment of autoimmunity, cancer, inflammatory disease, and/or transplantation.

### SUMMARY

In one aspect there is provided a method of treating a cancer in a subject, at risk of developing said cancer, or predisposed to said cancer, comprising: administering a therapeutically effective amount of PCLX-001.

As described herein, there is provided:

1. A method of treating a cancer in a subject, at risk of developing said cancer, or predisposed to said cancer, comprising: administering a therapeutically effective amount of PCLX-001.
2. The method of item 1, wherein said cancer is a lymphoma.
3. The method of item 2, wherein said lymphoma is B-cell lymphoma.
4. The method of any one of items 1 to 3, wherein said subject is a human.
5. Use of a therapeutically effective amount of PCLX-001 for treating a cancer in a subject, at risk of developing said cancer, or predisposed to said cancer.
6. Use of a therapeutically effective amount of PCLX-001 in the manufacture of a medicament for treating a cancer in a subject, at risk of developing said cancer, or predisposed to said cancer.
7. The use of item 5 or 6, wherein said cancer is a lymphoma.
8. The use of item 7, wherein said lymphoma is B-cell lymphoma.
9. The use of any one of items 5 to 8, wherein said subject is a human.
10. A method of inducing cell death of in a lymphoma is a subject, comprising: administering a therapeutically effective amount of PCLX-001 to said subject.
11. The method of item 10, wherein said lymphoma is B-cell lymphoma.
12. The method of item 10 or 11, wherein said subject is a human.
13. Use of a therapeutically effective amount of PCLX-001 for inducing lymphoma in a subject.
14. Use of a therapeutically effective amount of PCLX-001 in the manufacture of a medicament for inducing lymphoma in a subject.
15. The use of item 13 or 14, wherein said lymphoma is B-cell lymphoma.
16. The use of any one of items 13 to 15, wherein said subject is a human.
17. A method of reducing SFK protein levels or activity in a cell of a subject comprising: contacting said cell with PCLX-001.
18. The method of item 17, where said SFK protein is Src protein, Lyn protein, or both Src protein and Lyn protein.
19. The method of item 17 or 18, wherein said cell is a lymphoma cell.
26. The method of item 19, wherein said lymphoma is a B-cell lymphoma cell.
21. The method of any one of items 17 to 20, wherein said subject is a human.
22. The method of any one of items 17 to 21, wherein said contacting is in vitro or in vivo.
23. The method of any one of items 17 to 22, comprising a plurality of said cells.
24. Use of PCLX-001 for reducing SFK protein levels or activity in a cell of a subject, wherein said PCLX-001 is formulated for contacting with said cell.
25. Use of PCLX-001 in the manufacture of a medicament for reducing SFK protein levels or activity in a cell of a subject, wherein said PCLX-001 is formulated for contacting with said cell.
26. The use of item 24 or 25, wherein said SFK protein is Src protein, Lyn protein, or both Src protein and Lyn protein.
27. The use of any one of items 24 to 26, wherein said cell is a lymphoma cell.
28. The use of item 27, wherein said lymphoma is a B-cell lymphoma cell.
29. The use of any one of items 24 to 28, wherein said subject is a human.
30. The use of any one of items 24 to 29, wherein said contacting is in vitro or in vivo.
31. The use of any one of items 24 to 36, comprising a plurality of said cells.
32. A method of reducing one or more of Src protein, Lyn protein, pan-P-SFK protein, ERK protein, P-ERK protein, NFκB protein, c-Myc protein, or CREB protein, levels or activity in a cell of a subject, comprising: contacting said cell with PCLX-001.
33. The method of item 32, wherein said cell is a lymphoma cell.
34. The method of item 33, wherein in said lymphoma cell is a B-cell lymphoma.
35. The method of any one of items 32 to 34, wherein said subject is a human.
36. The method of any one of items 32 to 35, wherein said contacting is in vitro or in vivo.
37. The method of any one of items 32 to 36, comprising a plurality of said cells.
38. Use of PCLX-001 for reducing one or more of Src protein, Lyn protein, pan-P-SFK protein, ERK protein, P-ERK protein, NFκB protein, c-Myc protein, or CREB protein levels or activity in a cell of a subject, wherein said PCLX-001 is formulated for contacting with said cell.
39. Use of PCLX-001 in the manufacture of a medicament for reducing one or more of Src protein, Lyn protein, pan-P-SFK protein, ERK protein, P-ERK protein, NFκB protein, c-Myc protein, or CREB proteinlevels in a cell of a subject, wherein said PCLX-001 is formulated for contacting with said cell.
40. The use of item 38 or 39, wherein said cell is a lymphoma cell.
41. The use of item 40, wherein said lymphoma is a B-cell lymphoma cell.
42. The use of any one of items 38 to 41, wherein said subject is a human.
43. The use of any one of items 38 to 42, wherein said contacting is in vitro or in vivo.
44. The use of any one of items 38 to 43, comprising a plurality of said cells.
45. A method of treating an autoimmune disorder in a subject, comprising: administering a therapeutically effective amount of PCLX-001.
46. A method of treating an autoimmune disorder in a subject, comprising: administering a therapeutically effective amount of DDD85646.
47. A method of treating an autoimmune disorder in a subject, comprising: administering a therapeutically effective amount of IMP 1008.
48. A method of treating an autoimmune disorder in a subject, comprising: administering a therapeutically effective amount of an NMT inhibitor.
49. The method of any one of items 45 to 48, wherein said autoimmune disorder is rheumatoid arthritis, asthma, multiple sclerosis, myasthenia gravis, lupus erythematosus, insulin-dependent diabetes (type 1), gastritis, colitis, and insulin-dependent autoimmune diabetes, graft transplant/inhibition of rejection, psoriasis, Sjogren's syndrome or graft vs host disease.
56. The method of any one of items 45 to 49, wherein the subject is a human. 51. A method of treating an inflammatory disorder in a subject, comprising: administering a therapeutically effective amount of PCLX-001.
52. A method of treating an inflammatory disorder in a subject, comprising: administering a therapeutically effective amount of DDD85646.
53. A method of treating an inflammatory disorder in a subject, comprising: administering a therapeutically effective amount of IMP 1008
54. A method of treating an inflammatory disorder in a subject, comprising: administering a therapeutically effective amount of an NMT inhibitor.
55. The method of any one of items 51 to 54, wherein said inflammatory disorder is acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, ulcerative inflammation, a gastrointestinal disorder, a peptic ulcer, a regional enteritis, diverticulitis, gastrointestinal bleeding, eosinophilic, eosinophilic esophagitis, eosinophilic gastritis, eosinophilic gastroenteritis, eosinophilic colitis, gastritis, diarrhea, gastroesophageal reflux disease (GORD, or GERD), inflammatory bowel disease (IBD), Crohn's disease, ulcerative colitis, collagenous colitis, lymphocytic colitis, ischaemic colitis, diversion colitis, Behcet's syndrome, indeterminate colitis, inflammatory bowel syndrome (IBS), or a disorder of the lung selected from pleurisy, alveolitis, vasculitis, pneumonia, chronic bronchitis, bronchiectasis, diffuse panbronchiolitis, hypersensitivity pneumonitis, asthma, idiopathic pulmonary fibrosis (IPF), and cystic fibrosis.
56. The method of any one of items 51 to 55, wherein said subject is a human.
57. A method of reducing a BCR protein level or activity and/or TCR protein level or activity in a cell of a subject, comprising: contacting said cell with PCLX-001.
58. A method of reducing a BCR protein level or activity and/or TCR protein level or activity in a cell of a subject, comprising: contacting said cell with DDD85646.
59. A method of reducing a BCR protein level or activity and/or TCR protein level or activity in a cell of a subject, comprising: contacting said cell with and NMT inhibitor.
60. The method of any one of items 57 to 59, wherein said subject is a human.
61. The method of any one of items 57 to 60, wherein said contacting is in vitro or in vivo.
62. The method of any one of items 57 to 61, comprising a plurality of said cells.
63. A method reducing the activity of an immune cell from a subject, comprising: contacting said T-cell and/or said B-cell with an NMT inhibitor.
64. A method of reducing the activity of a T-cell and/or a B-cell from a subject, comprising: contacting said T-cell and/or said B-cell with an NMT inhibitor.
65. The method of item 63 or 64, wherein said NMT inhibitor is PCLX-001.
66. The method of item 63 or 64, wherein said NMT inhibitor is DDD85646.
67. The method of item 63 or 64, wherein said NMT inhibitor is IMP 1088.
68. The method of any one of items 63 to 67, wherein said subject is a human,
69. The method of any one of items 63 to 68, wherein said contacting is in vitro or in vivo.
70. Use of a therapeutically effective amount of PCLX-001 for treating an autoimmune disorder in a subject.
71. Use of a therapeutically effective amount of DDD85646 for treating an autoimmune disorder in a subject.
72. Use of a therapeutically effective amount of IMP 1088 for treating an autoimmune disorder in a subject.
73. Use of a therapeutically effective amount of an NMT inhibitor for treating an autoimmune disorder in a subject.
74. The use of any one of items 6 3to 73, wherein said autoimmune disorder is rheumatoid arthritis, asthma, multiple sclerosis, myasthenia gravis, lupus erythematosus, insulin-dependent diabetes (type 1), gastritis, colitis, and insulin-dependent autoimmune diabetes, graft transplant/inhibition of rejection, or graft vs host disease.
75. The use of any one of items 63 to 74, wherein the subject is a human.
76. Use of a therapeutically effective amount of PCLX-001 for treating an inflammatory disorder in a subject.
77. Use of a therapeutically effective amount of DDD85646 for treating an inflammatory disorder in a subject.
78. Use of a therapeutically effective amount of IMP 1088 for treating an inflammatory disorder in a subject.
79. Use of a therapeutically effective amount of an NMT inhibitor for treating an inflammatory disorder in a subject.
80. The use of any one of items 76 to 79, wherein said inflammatory disorder is acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, ulcerative inflammation, a gastrointestinal disorder, a peptic ulcer, a regional enteritis, diverticulitis, gastrointestinal bleeding, eosinophilic, eosinophilic esophagitis, eosinophilic gastritis, eosinophilic gastroenteritis, eosinophilic colitis, gastritis, diarrhea, gastroesophageal reflux disease (GORD, or GERD), inflammatory bowel disease (IBD), Crohn's disease, ulcerative colitis, collagenous colitis, lymphocytic colitis, ischaemic colitis, diversion colitis, Behcet's syndrome, indeterminate colitis, inflammatory bowel syndrome (IBS), or a disorder of the lung selected from pleurisy, alveolitis, vasculitis, pneumonia, chronic bronchitis, bronchiectasis, diffuse panbronchiolitis, hypersensitivity pneumonitis, asthma, idiopathic pulmonary fibrosis (IPF), Sjogren's syndrome and cystic fibrosis.
81. The use of any one of items 76 to 80, wherein said subject is a human.
82. Use of a therapeutically effective amount of PCLX-001 for reducing a BCR protein level or activity and/or TCR protein level or activity in a cell of a subject.
83. Use of a therapeutically effective amount of DDD85646 for reducing a BCR protein level or activity and/or TCR protein level or activity in a cell of a subject.
84. The use of a therapeutically effective amount of NMT inhibitor for reducing a BCR protein level or activity and/or TCR protein level or activity in a cell of a subject.
85. The use of any one of items 82 to 84, wherein said subject is a human.
86. The use of any one of items 82 to 85, wherein said contacting is in vitro or in vivo.
87. The use of any one of items 82 to 86, comprising a plurality of said cells.
88. A use of an NMT inhibitor, for reducing the activity of an immune cell from a subject.
89. A use of an NMT inhibitor for reducing the activity of a T-cell and/or a B-cell from a subject.
90. The use of item 87 or 89, wherein said NMT inhibitor is PCLX-001.
91. The use of item 87 or 89, wherein said NMT inhibitor is DDD85646.
92. The use of items 87 or 89, wherein said NMT inhibitor is IMP 1088.
93. The use of any one of items 87 to 89, wherein said subject is a human,
94. The use of any one of items 87 to 93, wherein said contacting is in vitro or in vivo.
95. A method reducing the activity of a monocyte cell in a subject or reducing the number of monocyte cells in a subject, comprising: contacting said monocyte with an NMT inhibitor.
96. The method of item 95, wherein said NMT inhibitor is PCLX-001.
97. The method of item 95, wherein said NMT inhibitor is DDD85646.
98. The method of item 95, wherein said NMT inhibitor is IMP 1088.
99. The method of any one of items 95 to 98, wherein said subject is a human,
100. The method of any one of items 95 to 99, wherein said contacting is in vitro or in vivo.
101. Use of an NMT inhibitor for reducing the activity of a monocyte cell in a subject or reducing the number of monocyte cells in a subject.
102. The use of item 101, wherein said NMT inhibitor is PCLX-001.
103. The use of item 101, wherein said NMT inhibitor is DDD85646.
104. The use of item 101, wherein said NMT inhibitor is IMP 1088.
105. The use of any one of items 101 to 104, wherein said subject is a human,
106. The use of any one of items 101 to 105, wherein said contacting is in vitro or in vivo
107. A method of reducing the amount of cytokine secretion in a T-cell in a subject, comprising: administering an NMT inhibitor.
108. The method of of item 107, wherein said cytokine is IL-6, IL-8 and IFN-gamma. IL-5, IL-10, or IL-13.
109. The method of item 107 or 108, wherein said NMT inhibito is PCLX-001.
110. The method of item 107 or 108, wherein said NMT inhibitor is DDD85646.
111. The method of item 107 or 108, wherein said NMT inhibitor is IMP-1088.
112. The method of any one of items 107 to 111, wherein said subject is a human,
113. The method of any one of items 107 to 112, wherein said contacting is in vitro or in vivo.
114. Use of an NMT inhibitor for reducing the amout of cytokine secretion in a T-cell in a subject.
115. The use of of item 114, wherein said cytokine is IL-6, IL-8 and IFN-gamma. IL-5, IL-10, or IL-13.
116. The use of item 114 or 115, wherein said NMT inhibito is PCLX-001.
117. The use of item 114 or 115, wherein said NMT inhibitor is DDD85646.
118. The use of item 114 or 115, wherein said NMT inhibitor is IMP-1088.
119. The method of any one of items 114 to 118, wherein said subject is a human,
120. The method of any one of items 114 to 119, wherein said contacting is in vitro or in vivo.

### BRIEF DESCRIPTION OF THE FIGURES

Embodiments of the present disclosure will now be described, by way of example only, with reference to the attached Figures.

FIG. 1A-H. PCLX-001 selectively kills hematological cancer cell lines in comparison to cancer cell lines of other origins. Percentage of maximum growth inhibition of various cell lines following 96 hr treatment with 1.21M PCLX-001 as determined using a Horizon cell line screen (A, B), or following 72 hr treatment with 1 μM of PCLX-001 using a Oncolines™ cell line screen (C, D). Cell lines are arranged according to tumor cell type. Cross-hatched zone represents cytotoxic effect. Hematological cancer cell lines are depicted in grey while all other types of cancer cell lines are depicted in white. Corresponding violin graphs compare the average PCLX-001-mediated growth inhibition on hematological cancer cell lines to cancer cell lines of other origins combined as calculated from the Horizon (B) and Oncolines™ (D) cell screens (Unpaired t-test, two-tailed P<0.0001). Quartiles are separated by dotted lines. Error bars represent standard deviation within each group. Normalized cell viability curves of immortalized lymphocyte (IM9, VDS), BL (BL2, Ramos, BJAB), and DLBCL (DOHH2, WSU-DLCL2, SU-DHL-10) cell lines treated with 0.001-5 μM of PCLX-001 for 96 hrs as determined by CellTiter Blue Viability Assay (E). Corresponding histograms of absolute IC54 values derived from cell viability curves plotted in E (F). (***) indicates a significant difference (P≤0.001) in IC50 between IM9 cells and all other cell lines tested (Ordinary one-way Anova, Tukey's multiple comparisons test, P<0.0001). Normalized proliferation of IM9 (G) and BL2 (H) cells reated with 0-5 μM of PCLX-001 for 96 hrs as determined by cell count. Values are mean±s.e.m. of 3 independent experiments.

FIG. 2A-G. PCLX-001 selectively inhibits myristoylation in vitro and induces apoptosis in lymphoma cell lines. Click chemistry was used on alkyne-myristate labelled cell lysates to determine overall protein myristoylation levels in: BL2 cells (A) and IM9 cells (B) treated for 1 hr with 0.01-1.0 μM PCLX-001, myristoylation levels of a WT-Src-EGFP construct expressed in COS-7 cells (C) and, myristoylation of immunoprecipitated endogenous pp60-Src in IM9 cells following 1 hr treatment with 1.0-10 μM of PCLX-001 (D). Fluorescence micrographs of COS-7 cells transfected with a WT-Src-EGFP (left), non-myristoylatable G2A-Src-EGFP mutant (centre), and a WT-Src-EGFP construct treated with 10 μM PCLX-001 for 24 hrs (right) (E). Scale bars are equal to 10 μm. Endogenous Src protein levels in IM9 and BL2 cells treated with 1 μM PCLX-001 for 0-5 days measured by Western blotting (F). Western blotting of cleaved PARP-1 and cleaved caspase-3 in IM9, BL2 and Ramos cell lysates following 72 hr incubation with 0-1.0 μM PCLX-001 (composite gels) (G). All data shown are representative of at least three independent experiments. GAPDH serves as loading control

FIG. 3A,B. PCLX-001 treatment results in SFK instability and degradation by the proteasome in lymphoma cell lines. Western blot for total Src and Lyn proteins in immortalized lymphocyte (IM9, VDS), BL (BL2, Ramos, BJAB), and DLBCL (DOHH2, WSU-DLCL2, SU-DHL-10) cell line lysates following 24-48 hrs of treatment with 0.1 μM or 1.0 μM PCLX-001 (A). Western blot for total Src, Lyn, Hck, Lck, Mcl-1, total phospho-tyrosine (PY99) and pan phosphorylated-SFK (P-SFK) protein levels in BL2 treated for 24-48 hrs with 1 μM PCLX-001 in the presence or absence of 10 μM of the proteasome inhibitor MG132 for the last 6 hrs (B). GAPDH serves as a loading control. All western blots shown are representative of three independent experiments.

FIG. 4A-C. PCLX-001 treatment attenuates BCR downstream signaling events in BL2 lymphoma cells. Western blot of BL2 cells treated for 48 hrs with 0.1 μM or 1.0 μM of dasatinib, ibrutinib or PCLX-001 to detect total tyrosine phosphorylation (P-Tyr), Lyn, Lyn phosphorylated on tyrosine 396 or 507, BTK, and BTK phosphorylated on tyrosines 223 or 551 (A), HGAL, SYK, phosphorylated SYK (P-SYK) (B) or ERK, phosphorylated ERK (P-ERK), NFκB, c-Myc, CREB, Arf-1, BIP and PARP-1 (C). Western blots are representative of at least three independent experiments. GAPDH serves as a loading control. BL2 cells were activated with 25 μg/ml F(ab′)₂ anti-human IgM for 2 min and processed for western blotting. All western blots shown are representative of three independent experiments.

FIG. 5A,B. Model depicting proposed PCLX-001 mechanism of action in B cell lymphoma. (A) Upon BCR activation, first the myristoylated SFK Lyn is recruited to the lipid raft domains of the plasma membrane containing the BCR, dephosphorylated Lyn at Y507 leads to its activation and autophosphorylation at Y396. This leads to the phosphorylation and activation of BTK at Y551 and Y223. Second, myristoylated HGAL is also recruited to the plasma membrane and phosphorylated thereby enhancing BCR signaling by stimulating SYK, BTK and the release of Ca⁺⁺ ions from the endoplasmic reticulum via the inositol-3-phosphate ion channel receptor (IP3R). Altogether these early signaling events lead to transcription activation by c-Myc, P-ERK, NFκB, and CREB. (B) The NMT inhibitor PCLX-001 prevents the myristoylation of Lyn-SFK (as well as other SFKs not shown in this model), HGAL and Arf1 thereby impeding the proper membrane targeting and function of these proteins. PCLX-001 treatment impedes calcium homeostasis by reducing the BCR mediated Ca⁺⁺ release from the ER and increasing basal Ca⁺⁺ levels in cells in addition to promote the degradation of both myristoylated (Lyn, HGAL, Arf1) and, surprisingly, non-myristoylated proteins (NFκB, P-ERK, c-Myc and CREB), some via the ubiquitin-proteasome pathway thereby further abrogating downstream BCR signaling and increasing ER stress leading to apoptosis and cell death.

FIG. 6A-D. PCLX-111 selectively kills hematological cancer cells relative to benign lymphocytes in comparison to dasatinib and ibrutinib. Cell viability curves of BL2 (solid lines) and IM9 cells (dotted lines) treated for 48 hrs (A) or 96 hrs (B) with 0.001-5 μM dasatinib, ibrutinib, or PCLX-61. Normalized cell viability of immortalized lymphocyte (IM9, VDS), BL (BL2, Ramos, BJAB), and DLBCL (DOHH2, WSU-DLCL2, SU-DHL-16) cell lines treated with 0.1 μM or 1.0 μM of dasatinib, ibrutinib or PCLX-001 for 48 hrs (C) and 96 hrs (D). Cell viability for all experiments was measured using Calcein assay and is an average of three independent experiments. Errors bars depict s.e.m.

FIG. 7A-G. PCLX-001 treatment reduces tumor volumes and leads to complete tumor regression in B-cell lymphoma xenograft models. Dose-response curves for murine subcutaneous xenografts derived from cell lines measuring the size of DOHH2 (A) and BL2 (B) tumors as a function of time. Error bars represent the standard deviation of average tumor volumes. Average total NMT specific activity assessed as previously described²¹ in BL2 tumor samples from mice treated with PCLX-001, doxorubicin, or vehicle alone at the indicated doses. Tumor extracted from mice treated with 60 mg/kg/day have reduced NMT specific activity as compared to vehicle (paired t-test, P=0.0425). Error bars represent s.e.m. (C). Dose-response curve for the murine xenograft derived from patient DLBCL3. Data points represent average tumor volumes in all surviving animals. Error bars represent the standard deviation in the average tumor volumes (D). (***) indicates a significant difference in response rate between animals which received 20 mg/kg/day and 50 mg/kg/day of PCLX-001 (P<0.0001). Representative tumors from mice with patient-derived DLBCL3 xenografts (E). Representative IHC staining for cleaved caspase-3 (F) and Ki-67 (G) in the above DLBCL3 patient xenograft tumor samples. Scale bars equal to 100 μm.

FIG. 8. Combined Horizon and Oncoline cell line screen data demonstrates that PCLX-001 confers maximal growth inhibition on hematologic cancer cell lines in comparison to cell lines derived from all other cancer types. Violin graph depicting the combined percentage growth inhibition of PCLX-001 on hematological cell lines versus all other non-hematological cell lines from both the Horizon and Oncoline cell line screens following 96 hrs of treatment. Quartiles are separated by dotted lines. (***) indicates a significant difference in growth inhibition (Unpaired t-test, two-tailed P<0.0001).

FIG. 9A-D. Breadth of efficacy screen demonstrates that PCLX-001 is active against various other cancer cell lines including those derived from solid tumors. Absolute IC50 values of various cell lines treated for 3 days (A, B) or 6 days (C, D) with 0.0005-10 μM PCLX-001. Cell lines are arranged according cancer type. Individual bars represent a single cancer cell line derived from B cell lymphoma and Mantle Cell Lymphoma, Acute Myeloid Leukemia (AML), Breast, Small-cell lung carcinoma (SCLC), Non-small-cell lung carcinoma (NSCLC). ChemPartner robotic platform determined cell viability using CellTiter Blue viability assay. Growth inhibition (GI) was not calculated since the viability of the cells at Day 0 was not available from the Chempartner platform. (Unpaired t-test, two-tailed, ** P=0.0038, ns=non significant).

FIG. 10A,B. PCLX-001 selectively kills hematological cancer cell lines in comparison to immortalized lymphocytes. (A) Normalized cell viability curves of immortalized lymphocytes (IM9, VDS), BL (BL2, Ramos, BJAB), and DLBCL (DOHH2, WSU-DLCL2, SU-DHL-10) cell lines treated with 0.00-5 μM of PCLX-001 for 96 hrs, as determined by Calcein Assay, which measures the percentage of viable cells regardless of the number of cells. (B) Corresponding histograms of absolute IC₅₀ values derived from cell viability curves plotted in (A). Values are mean±s.e.m. of 3 experiments. (Ordinary one-way Anova, Tukey's multiple comparisons test, *** P<0.0001).

FIG. 11A-C. PCLX-001 treatment decreases the normalized lymphoma cell line proliferation. (A) Normalized proliferation of immortalized lymphocyte (VDS), BL (Ramos, BJAB), and DLBCL (DOHH2, WSU-DLCL2, SU-DHL-1S) cell lines treated with 0-5 μM of PCLX-001 for 96 hrs as determined by cell count. (B) Inhibition of the normalized proliferation of various cell lines after 0.1 μM PCLX-001 treatment up to 96 hrs. (C) To account for the differences in cell growth rates were transformed our data into a relative ratio of the normalized proliferation of various cell lines after 0.1 μM PCLX-001 treatment up to 96 hrs divided by the normalized proliferation of the respective untreated cell lines. Values are mean±s.e.m. of 3 experiments.

FIG. 12A,B. A large proportion of freshly isolated human lymphocytes, PBMCs and primary umbilical vein endothelial cells (HUVEC) are resistant to PCLX-001. Cell viability curve of 2 freshly isolated human peripheral blood monocytes (PBMC) and lymphocytes preparations treated for 96 hrs with 0.001-10 μM PCLX-001. Values are mean±s.e.m. (n=2). HUVECs were treated for 96 hrs with 0.001-5 μM PCLX-001 and residual cell viability was determined using a Cell-Titer Blue Assay. Values are mean±S.D. (n=4).

FIG. 13A,B. PCLX-001 does not inhibit palmitoylation of Ras and does not have any significant off-target kinase inhibitor activity at physiological level. (A) COS-7 cells transiently expressing palmitoylatable EGFP-N-Ras or non-palmitoylatable EGFP-K-Ras for 48 hrs were pre-treated for 1 hr with 100 μM 2-bromopalmitate (2-BP), a palmitoylation inhibitor or the following NMT inhibitors: 10 μM PCLX-001, 100 μM 2-hydroxymyristate (HMA) or 10 μM Tris-DBA. The cells pre-treated with inhibitors were then labelled for 4 hrs with 100 μM Alkyne-C16. EGFP-tagged constructs were immunoprecipitated as described and reacted with azido-biotin using click chemistry. Biotinylated-palmitoylated proteins were detected using neutravidin-HRP conjugate and ECL. (B) TREEspot™ is a proprietary data visualization software tool developed by DiscoverX Corporation, CA, USA. 468 pre-configured human kinases of the scanMAX KINOMEscan were tested. Mutant and lipid kinases are not represented. Possible kinases found to bind PCLX-001 are marked with red circles, where larger circles indicate higher-affinity binding. No kinases were found binding with PCLX-001 up to 10 μM, which corresponds to a ˜400 times larger concentration than the PCLX-001 EC50 for BL2 cells. At 100 μM PCLX-001 (˜4000 times the EC50 for BL2), only 3 kinases (MRCKA, PIP5K2C and SRPK1 shown in red) were found to weakly bind PCLX-001 (kinase activity score <35% of control). All western blots shown are representative of three independent experiments.

FIG. 14. Quantification of the Src protein level decrease in BL2 and IM9 cells treated with PCLX-001 for up to 5 days. Quantification of total endogenous Src protein levels detected by Western blot (FIG. 2F). Errors bars depict standard error from the mean. (*) indicates a significant difference (2way ANOVA, P=0.0174) in Src protein levels (n=3).

FIG. 15A,B. PCLX-001 treatment reduces phospho-tyrosine levels in basal (tonic or chronic) and anti-IgM activated signaling in various normal and malignant B cell lines. (A) Western blots assessing the basal (antigen independent tonic or chronic) tyrosine phosphorylation levels (PY99) in normal IM9 and VDS cell lines, and malignant B cell lines BL2, Ramos, BJAB, DOHH2, WSU-DLCL2 and SU-DHL-10 cells following 24 hrs treatment with 0.01-1 μM PCLX-001. (B) PY99 Western blot of unstimulated (left) and anti-IgM ligated BCR (right) BL2 cells treated for 24 hrs with 0.1 μM or 1 μM of dasatinib, ibrutinib or PCLX-001. BL2 cells were activated as indicated with 25 μg/ml goat anti-human IgM for 2 min. Western blots shown are representative of at least 3 independent experiments.

FIG. 16A-C. PCLX-001 treatment significantly decreases total phospho-tyrosine, phospho-ERK (P-ERK) and NFκB levels in BL2 cells. Quantification of western blots for total phospho-tyrosine levels using PY99 antibody (A), P-ERK (B) and NFκB (C) in BL2 cells treated for 48 hrs with 0.1 μM or 1.0 μM of dasatinib, ibrutinib or PCLX-001 (FIG. 4A) (n=4 for A and C, n=3 for B). BL2 cells were activated with 25 μg/ml goat anti-human IgM for 2 min where indicated. Errors bars depict standard error from the mean. (*) indicates a significant difference (P<0.05) in phosphor-tyrosine levels (Ordinary one-way Anova, Tukey's multiple comparisons test).

FIG. 17A,B. PCLX-001 treatment attenuates anti-IgM ligated BCR signaling in various lymphoma cell lines. Western blots of (A) BL (Ramos, BJAB), and (B) DLBCL (DOHH2, WSU-DLCL2, SUDHL-10) cell lines treated for 48 hrs with 0.1 μM or 1.0 μM of dasatinib, ibrutinib or PCLX-001 to detect total Src, Lyn, ERK, phosphorylated SFKs (P-SFK) and phosphorylated ERK (P-ERK) levels. Src and Lyn were not detected in DOHH2. Western blots are representative of at least three independent experiments. GAPDH serves as a loading control. Cell lines were activated with 25 μg/mL goat anti-human IgM for 2 min prior to Western blotting. All western blots shown are representative of three independent experiments.

FIG. 18A,B. Comparison of various SFK levels in BL2 cells following 48 hr treatment with PCLX-001, dasatinib, ibrutinib. Western blot (A) and quantification (B) of the protein levels of Lyn, Src, Lck, Hck, Fyn, and total phosphorylated SFKs (P-SFK blot is shown in FIG. 4A) in BL2 cells treated for 48 hrs with 0.1 μM or 1.0 μM of dasatinib, ibrutinib or PCLX-001. BL2 cells were activated with 25 μg/ml goat anti-human IgM for 2 min where indicated. Errors bars depict standard error from the mean. (***) indicates a significant difference (p<0.0001 in protein or phosphorylated protein levels (Ordinary one-way ANOVA, Tukey's multiple comparison test).

FIG. 19A-C. PCLX-001 reduces BCR receptor-dependent calcium release activated by antiIgM stimulation in BL2 cells. Endoplasmic reticulum Ca⁺⁺ release was measured in BL2 cells treated with 1 μM PCLX-001(A), Dasatinib (B) or Ibrutinib (C) for 24 h or 48 h. Following cell loading with the fluorescent Ca⁺⁺ indicator Fura-2 cells were stimulated with 10 μg/ml Goat F(ab′)2 anti-human IgM to ligate and activate BCR-receptor dependent Ca⁺⁺ release then following thapsigargin (300 nM) treatment to show BCR-receptor independent Ca⁺⁺ release from endoplasmic reticulum. Results shown are representative of multiple replicates of the experiment (n=6 for PCLX-001 incubation, n=3 for dasatinib and ibrutinib).

FIG. 20A,B. Dasatinib and ibrutinib do not synergize the cytotoxic effects of PCLX-001 in IM9 and BL2 cells. IM9 (A) and BL2 (B) cells were incubated with 0.01, 0.1 and 1 μM PCLX-001 in combination with 0.1 and 1 μM dasatinib or ibrutinib for 96 hours. No additive or synergistic effects were observed upon the addition of dasatinib or ibrutinin to PCLX-001. As seen throughout our experiments, malignant BL2 cells are more sensitive to PCLX-001 than normal IM9 B cells. Cell viability was measured using calcein assay and represents an average of three independent experiments. Errors bars depict s.e.m.

FIG. 21A-G. Influence of PCLX-001 and doxorubicin treatment on body weight and percentage survival in xenograft models. Percentage change in body weight in DOHH2 (A), BL2 (C), and (F) DLBCL3-patient derived xenograft models. Black arrows represent injections. Error bars represent the standard deviation in the average weight per mouse at each time point. Kaplan-Meier curves, where survival events include death from toxicity, death from cancer, or euthanasia for toxicity, depicting percent survival over time in (B) DOHH2, (D) BL2, and (G) DLBCL3-patient derived xenograft models. (E) Median survival estimates derived from Kaplan-Meier curve analysis of BL2 xenograft animals (D) following treatment with the indicated dosages of PCLX-001 and doxorubicin.

FIG. 22A-E. NMT expression is decreased in hematological cancer cell lines. The average number of NMTltranscripts is larger than NMT2 transcripts. However, NMT2 transcript numbers (grey) show larger variations than NMT1 transcript numbers (black) in cancer cell lines (A). NMT2 mRNA expression is significantly lower in hematological cancer cell lines (Unpaired t-test; *** P<0.0001) in comparison to cell lines originating from other types of cancers (Min to Max Box Plot, B). Expression of NMT1 (C) is relatively constant across the 1269 cell lines investigated with a slight but significant decrease in expression in breast and leukemia cancer cell lines while NMT2 expression (D) varies significantly amongst various cancers and also within a given cancer type. The data also illustrate that while the expression of NMT2 is higher in cancer cell lines of CNS, kidney and fibroblast origins there is a selective and significant reduction of NMT2 expression in hematological cancers such as leukemia, lymphoma and myeloma. Box plots are showing 10-90 percentiles (Ordinary one-way ANOVA, Dunnett's multiple comparisons test, *** P<0.0001). NMT1 expression is not increased in the 100 cells lines expressing the least NMT2 as a possible compensatory mechanism (E). All data were extracted from 20Q1 PublicRNA-sequencing (Broad Institute, 1269 cell lines) and sorted in a selection of cancers.

FIG. 23. PCLX-001 treatment attenuates TCR dependent P-ERK activation in Jurkat T cells. Jurkat T cells were activated with CD3/CD28 antibodies for up to 66 minutes (2 ug/ml). Immunoblotting analysis shows that PCLX-001 incubated for 24/48 h (1 μM) inhibit P-ERK activation.

FIG. 24. PCLX-001 treatment (24 h) attenuates TCR dependent P-ERK and P-SFK activation in Jurkat T cells. Jurkat T cells were activated with CD3/CD28 antibodies for up to 4 hours (2 ug/ml). Immunoblotting analysis shows that PCLX-001 incubated for 24 h (0.1 and 1 μM) P-ERK activation and phosphorylation of Src family kinases (P-SFK).

FIG. 25. PCLX-001 treatment (48 h) attenuates TCR dependent P-ERK and P-SFK activation in Jurkat T cells. Jurkat T cells were activated with CD3/CD28 antibodies for up to 4 hours (2 ug/ml). Immunoblotting analysis shows that PCLX-001 incubated for 48 h (0.1 and 1 μM) inhibit P-ERK activation and phosphorylation of Src family kinases (P-SFK).

FIG. 26. PCLX-001 and Dasatinib treatment attenuates TCR downstream signaling events and induce ER stress in primary cultured T cells. 96% ab primary T cells were activated with CD3/CD28 antibodies for 30 min (2 ug/ml). Immunoblotting analysis shows that PCLX-001 and Dasatinib inhibit P-tyrosine phosphorylation (PY99), P-ERK activation, phosphorylation of Src family kinases (P-SFK). In addition, PCLX-001 reduced the protein level of Src and Lyn significantly and increased Bip protein content (ER stress marker).

FIG. 27A-E. PCLX-001 reduces the viability of PBMC, B cells and monocytes but not T cells. PBMC were cultured for 4 days in the presence of increasing concentrations of PCLX-001 (0-10 ug/ml). the viability and the abundance of cell subset were tested using flow cytometry. The viability of PBMC was markedly reduced (A). Although the frequency of CD4+ and CD8+ T cells was not changed by the drug treatment (B and C). However, B cells (D) and monocyte CD14+ (E) numbers were significantly decreased after 96 hours of PCLX-001 treatment.

FIG. 28A-D. PCLX-001 reduces the expression of Lyn and HGAL in T cells. PBMC were cultured for 4 days in the presence of increasing concentrations of PCLX-001 (0-10 ug/ml). The expression of Lyn and HGAL in T cell subset were tested using intracellular staining through flow cytometry. The expression of Lyn (A) and HGAL (B) in CD4+ T cells were both decreased. In addition, PCLX-001 also reduced the expression of both Lyn (C) and HGAL (D) in CD8+ T cells.

FIG. 29A-D. PCLX-001 reduces the expression of Lyn and HGAL in monocytes but not in B cells. PBMC were cultured for 4 days in the presence of increasing concentrations of PCLX-001 (0-10 ug/ml). The expression of Lyn and HGAL in B cells and monocyte subset were tested using intracellular staining through flow cytometry. Although PCLX-661 couldn't reduce the expression of Lyn (A) and HGAL (B) in B cells, both protein markers were significantly reduced in monocytes (C and D).

FIG. 30A-E. PCLX-001 induces the production of inflammatory cytokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory cytokines IL-6 (A), TNF-α (B), IL-8 (C), IFN-γ (D), and IL-17a (E) in live PBMC.

FIG. 31A-D. PCLX-001 induces the production anti-inflammatory cytokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the anti-inflammatory cytokines IL-1RA (A), IL-10 (B), IL-13 (C), and IL-16 (D) in live PBMC.

FIG. 32A-D. PCLX-001 induces the production of inflammatory chemokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory chemokines MIP-1a (A), MCP-2 (B), TARC (C), and GRO-α (D) in live PBMC.

FIG. 33A-D. PCLX-001 induce sthe production of inflammatory chemokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory chemokines RATES (A), MIP-1β (B), MCP-4 (C), and MDC (D) live PB<C.

FIG. 34A-C. PCLX-001 induces the production of T helper 2-mediated chemokines and GM-CSF. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the granulocyte-monocyte colony stimulating factor I-349 (A), Eotaxin-2 (B) as T helper 2 mediated chemokines and GM-CSF (C) in live PBMC.

FIG. 35A-D. NMT inhibitors (PCLX-001, PCLX-002, IMP-1088) reduce the normalized secretion of pro-inflammatory cytokines; IL-6 (A), IL-8 (B), TNF-α (C), and IFN-γ (D). T cells were incubated for 48 h with increasing concentration of NMT inhibitors, then induced by T cell activator (STEMCELLS) in the presence of the drugs for 2 more days. NMT inhibitors significantly reduced the level of IL-6, IL-8 and IFN-gamma. (Two-way ANOVA, P value against untreated: *<0.05-0.01 **<0.01-0.001 ***<0.001-0.0001. It is noteworthy to mention that reduction of cytokine secretion is stronger in the more potent NMT inhibitor PCLX-001 than PCLX-002 and that the survival of cells after 4 days of treatment was within 10% of untreated samples.

FIG. 36A-D. NMT inhibitors (PCLX-001, PCLX-002, IMP-1088) reduce the normalized secretion of anti-inflammatory cytokines; IL-4 (A), IL-5 (B), IL-10 (C), and IL-13 (D). T cells were incubated for 48 h with increasing concentration of NMT inhibitors, then induced by T cell activator (STEMCELLS) in the presence of the drugs for 2 more days. NMT inhibitors significantly reduced the level of IL-5, IL-10 and IL-13.(Two-way ANOVA, P value against untreated: *<0.05-0.01 **<0.01-0.001 ***<0.001-0.0001. It is noteworthy to mention that reduction of cytokine secretion is stronger in the more potent NMT inhibitor PCLX-001 than PCLX-002 and that the survival of cells after 4 days of treatment was within 10% of untreated samples.

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

In one aspect described herein, we tested the sensitivity of 300 cancer cell lines encompassing all major cancer types to NMT inhibition by PCLX-001 in three independent screens. PCLX-001 is an orally bioavailable derivative of the NMT inhibitor DDD85646, and is more selective and potent towards human NMTs (Table 1)³⁸. We demonstrate that PCLX-001 inhibits the viability and growth of hematological cancer cells in vitro more effectively than the inhibition of viability and growth of other cancer cell types or select normal cells. PCLX-001 disrupts early BCR-mediated survival signaling in several B-cell lymphoma cell lines and promotes the degradation of numerous myristoylated and non-myristoylated BCR effectors, triggering apoptosis. More importantly, PCLX-001 produces dose-dependent tumour regression and complete tumor regressions in 2 of 3 lymphoma murine xenograft models.

TABLE 1
Structure and basic comparison of DDD85646 (PCLX- [symbol]
2) and DDD86418 (PCLX- [symbol] 1) NMT inhibitors.

| | | | | DDD [symbol] | |
|---|---|---|---|---|---|
| | | DDD [symbol] | | (PCLX- [symbol] 1) | |
| Structure | | | | | |
| Human NMT IC [symbol] | | 4 | nM | <1 | nM |
| [symbol] hepatic | Mouse | [symbol] .6 | | <0.5 | |
| clearance | (mL/min/g) | | | | |
| | Rat (mL/min/g) | [symbol] .5 | | 1.0 | |
| | Human | 1.2 | | 0.7 | |
| | (mL/min/g) | | | | |
| Fraction of drug [symbol] to | | 0.11 [symbol] /0.17 [symbol] | | 0.067 | |
| plasma [symbol] (mouse/human) | | | | ( [symbol] ) | |
| Mouse IV | Clearance | 5 | [3-7] | 3 | [2-3] |
| | (mL/min/kg) | | | | |
| | Volume [symbol] drug | [symbol] .6 | [0.4- [symbol] .7] | 0.4 | [0.3- [symbol] .4] |
| | distribution at | | | | |
| | [symbol] state | | | | |
| | (L/kg) | | | | |
| | T [symbol] (hours) | 1.3 | [1.3-1.4] | 1.5 | [1.0-2.1] |
| Mouse [symbol] | Cmax (ng/mL) | 2 [symbol] | [2122-3755] | 112 [symbol] 1 | [ [symbol] -13416] |
| 10 mg/kg | Tmax (hours) | 0.25 | [0.25-2] | 2 | |
| | T [symbol] (hours) | 1.2 | [1.0-1.4] | 5.7 | [2.7- [symbol] ] |
| | Orally absorbed | 20 | [11-32] | [symbol] 3 | [51-100] |
| | drug (%) | | | | |

| | | |
|---|---|---|
| Blood:Brain ratio | [symbol] .08 | 0.04 |

[symbol]

[symbol]

indicates data missing or illegible when filed

The structure of PCLX-001, also known as DDD86481, is as follows.

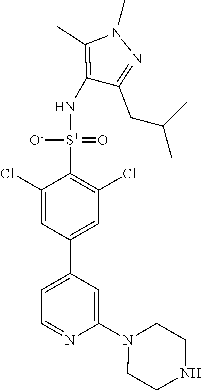 [SMILES: Cc1c(N[S+](=O)([O-])c2c(Cl)cc(-c3ccnc(N4CCNCC4)c3)cc2Cl)c(CC(C)C)nn1C]

The structure of DDD85646 (PCLX-002) is as follows.

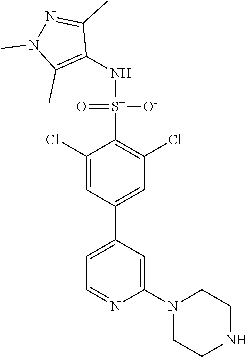 [SMILES: Cc1nn(C)c(C)c1N[S+](=O)([O-])c1c(Cl)cc(-c2ccnc(N3CCNCC3)c2)cc1Cl]

In another aspect described herein, PCLX-001 may be used as an anti-inflammatory agent.

In another aspect described herein, PCLX-001 may be used as an anti-autoimmune agent.

In one aspect, there is provided a method of treating a subject having a cancer, or suspected of having cancer, comprising: administering a therapeutically effective amount of PCLX-001. In a specific example, the cancer is a lymphoma. In a more specific example, the cancer is B-cell lymphoma.

In one aspect, there is provided a method of treating a subject having an inflammatory disease or disorder, or suspected of having an inflammatory disease or disorder, comprising: administering a therapeutically effective amount of PCLX-001. Thus, in some examples, PCLX-001 may be used as an anti-inflammatory agent.

In one aspect, there is provided a method of treating a subject having an auto-immune disease or disorder, or suspected of having an auto-immune disease or disorder, comprising: administering a therapeutically effective amount of PCLX-001. Thus, in some examples, PCLX-001 may be used as an anti-autoimmune agent.

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. Cancer cells may be in the form of a tumour, but such cells may also exist alone within a subject, or may be a non-tumorigenic cancer cell. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

It will be appreciated that, in general, determination of the severity of disease requires identification of certain disease characteristics, for example, whether the cancer is pre-metastatic or metastatic, the stage and/or grade of cancer, and the like.

Staging is a process used to describe how advanced a cancer is in a subject. Staging may be important in determining a prognosis, planning treatment and evaluating the results of such treatment. While different cancer staging systems may need to be used for different types of cancer, most staging systems generally involve describing how far the cancer has spread anatomically and attempt to put subjects with similar prognosis and treatment in the same staging group.

Examples of common staging systems used for most solid tumours, some leukemias and lymphomas are the Overall Stage Grouping system and the TMN system. In the Overall Stage Grouping system, Roman numerals I through IV are utilized to denote the four stages of a cancer. Generally, if a cancer is only detectable in the area of the primary lesion without having spread to any lymph nodes it is called Stage I. Stage II and III cancers are generally locally advanced and/or have spread to the local lymph nodes. For example, if the cancer is locally advanced and has spread only to the closest lymph nodes, it is called Stage II. In Stage III, the cancer is locally advanced and has generally spread to the lymph nodes in near proximity to the site of the primary lesion. Cancers that have metastasized from the primary tumour to a distant part of the body, such as the liver, bone, brain or another site, are called, Stage IV, the most advanced stage. Accordingly, stage I cancers are generally small localized cancers that are curable, while stage IV cancers usually represent inoperable or metastatic cancers. As with other staging systems, the prognosis for a given stage and treatment often depends on the type of cancer. For some cancers, classification into four prognostic groups is insufficient and the overall staging is further divided into subgroups. In contrast, some cancers may have fewer than four stage groupings.

A cancer that recurs after all visible tumour has been eradicated is called recurrent disease, with local recurrence occurring in the location of the primary tumour and distant recurrence representing distant metastasis.

Variations to the staging systems may depend on the type of cancer. Moreover, certain types of cancers. The staging system for individual cancers maybe revised with new information and subsequently, the resulting stage may change the prognosis and treatment for a specific cancer.

The “grade” of a cancer may be used to describe how closely a tumour resembles normal tissue of its same type. Based on the microscopic appearance of a tumour, pathologists identify the grade of a tumour based on parameters such as cell morphology, cellular organization, and other markers of differentiation. As a general rule, the grade of a tumour corresponds to its rate of growth or aggressiveness and tumours are typically classified from the least aggressive (Grade I) to the most aggressive (Grade IV).

Accordingly, the higher the grade, the more aggressive and faster growing the cancer. Information about tumour grade is useful in planning treatment and predicting prognosis.

In some examples, in the case of lymphoma, Stage 1 refers to lymphoma in only one group of lymph nodes. Stage II refers to two or more groups of lymph nodes are affected but they are all either above or below the diaphragm, either all in the chest or all in the abdomen. Stage III refers to two or more groups of lymph nodes are affected in both the chest and the abdomen. Stage IV refers to lymphoma is in at least one organ (e.g., bone marrow, liver or lungs) as well as the lymph nodes. Additional designations may be added to the foregoing stages. For example, “A” generally means the patient has not experiences any troublesome symptoms. “B” means the patient has experienced B symptoms (e.g., fever, night sweats, weight loss). X means the patient has bulky disease (e.g., large tumour greater than 10 cm in size). E means the patient has extranodal disease (e.g., disease outside the lymph nodes).

In a specific example, the cancer is a lymphoma.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyterich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NHL; cutaneous T-cell NHL; T-cell and natural killer cell NHL; mature (peripheral) T-cell NHL; Burkitt's lymphoma; mycosis fungoides; Sezary Syndrome; precursor B-lymophoblastic lymphoma; B-cell small lymphocytic lymphoma; lymphoplasmacytic lymphoma; spenic marginal zome B-cell lymphoma; nodal marginal zome lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic Tcell lymphoma; and primary systemic anaplastic large T/null cell lymphoma.

In a specific example, the lymphoma is a B-cell lymphoma.

In some examples, the compositions and/or compositions described herein (for example, PCLX-001) may be used to treat various stages and grades of cancer development and progression. In some examples, PCLX-001 may be used in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, PCLX-001 may be used in the treatment of low grade cancers, intermediate grade cancers and or high grade cancers.

In some examples, it is contemplated that PCLX-001 may be used in the treatment of indolent cancers, recurrent cancers including locally recurrent, distantly recurrent and/or refractory cancers (i.e., cancers that have not responded to treatment), metastatic cancers, locally advanced cancers and aggressive cancers.

In some examples, PCLX-001 may be used alone or in combination with one or more therapeutic agents as part of a primary therapy or an adjuvant therapy. “Primary therapy” or “first-line therapy” refers to treatment upon the initial diagnosis of cancer in a subject. Exemplary primary therapies may involve surgery, a wide range of chemotherapies, immunotherapy and/or radiotherapy. When first-line or primary therapy is not systemic chemotherapy or immunotherapy, then subsequent chemotherapy or immunotherapy may be considered as “first-line systemic therapy”. In one example, PCLX-001 may be used for first-line systemic therapy.

The term “adjuvant therapy” refers to a therapy that follows a primary therapy and that is administered to subjects at risk of relapsing. Adjuvant systemic therapy is typically begun soon after primary therapy to delay recurrence, prolong survival or cure a subject. Treatment of a refractory cancer may be termed a “second-line therapy” and is a contemplated use of the present invention, in addition to first-line therapy.

The term “sample” as used herein refers to any sample from a subject, including but not limited to a fluid, cell or tissue sample that comprises one or more cells, , which can be assayed for gene expression levels, proteins levels, enzymatic activity levels, and the like. The sample may include, for example, a blood sample, a fractionated blood sample, a bone marrow sample, a biopsy, a frozen tissue sample, a fresh tissue specimen, a cell sample, and/or a paraffin embedded section, material from which RNA can be extracted in sufficient quantities and with adequate quality to permit measurement of relative mRNA levels, or material from which polypeptides can be extracted in sufficient quantities and with adequate quality to permit measurement of relative polypeptide levels.

In one embodiment of the present invention, the combinations are used in the treatment of an early stage cancer. In another embodiment, the combinations are used as a first-line systemic therapy for an early stage cancer.

In an alternate example, PCLX-001 may be used in the treatment of a late stage and/or advanced and/or metastatic cancer. In a further embodiment, PCLX-001 may be adminstered as a first-line systemic therapy for the treatment of a late stage and/or advanced and/or metastatic cancer.

In a specific example, PCLX-001 may be used in the treatment of lymphoma. In a more specific example, PCLX-001 may be used in the treatment of B-cell lymphoma.

As shown herein, PCLX-001 inhibits the BCR, and thus may be used an anti-inflammatory agent, and/or may be used as an anti-autoimmune agent.

The term “immune cell” generally encompasses any cell derived from a hematopoietic stem cell that plays a role in the immune response. The term is intended to encompass immune cells both of the innate or adaptive immune system. The immune cell as referred to herein may be a leukocyte, at any stage of differentiation (e.g., a stem cell, a progenitor cell, a mature cell) or any activation stage. Immune cells include lymphocytes (such as natural killer cells, T-cells (including, e.g., thymocytes, Th or Tc; Th1, Th2, Th17, T1 [symbol] αβ, CD4+, CD8+, effector Th, memory Th, regulatory Th, CD4+/CD8+ thymocytes, CD4−/CD8− thymocytes, γδ T cells, etc.) or B-cells (including, e.g., pro-B cells, early pro-B cells, late pro-B cells, pre-B cells, large pre-B cells, small pre-B cells, immature or mature B-cells, producing antibodies of any isotype, T1 B-cells, T2, B-cells, naive B-cells, GC B-cells, plasmablasts, memory B-cells, plasma cells, follicular B-cells, marginal zone B-cells, B-1 cells, B-2 cells, regulatory B cells, etc.), such as for instance, monocytes (including, e.g., classical, non-classical, or intermediate monocytes), (segmented or banded) neutrophils, eosinophils, basophils, mast cells, histiocytes, microglia, including various subtypes, maturation, differentiation, or activation stages, such as for instance hematopoietic stem cells, myeloid progenitors, lymphoid progenitors, myeloblasts, promyelocytes, myelocytes, metamyelocytes, monoblasts, promonocytes, lymphoblasts, prolymphocytes, small lymphocytes, macrophages (including, e.g., Kupffer cells, stellate macrophages, M1 or M2 macrophages), (myeloid or lymphoid) dendritic cells (including, e.g., Langerhans cells, conventional or myeloid dendritic cells, plasmacytoid dendritic cells, mDC-1, mDC-2, Mo-DC, HP-DC, veiled cells), granulocytes, polymorphonuclear cells, antigen-presenting cells (APC), etc.

The term “B cell” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface.

The term “T cell” refers to a type of lymphocyte that matures in the thymus. T cells play an important role in cell-mediated immunity and are distinguished from other lymphocytes, such as B cells, by the presence of a T-cell receptor on the cell surface.

The B Cell Receptor (BCR) and The BCR Complex B cells are immune system cells that are responsible for producing antibodies. The B cell response to antigen is an essential component of the normal immune system. B cells possess specialized cell surface receptors (B cell receptors; “BCR”). If a B cell encounters an antigen capable of binding to that cell's BCR, the B cell will be stimulated to proliferate and produce antibodies specific for the bound antigen. To generate an efficient response to antigens, BCR associated proteins and T cell assistance are also required.

Signaling through the BCR plays an important role in the generation of antibodies, in autoimmunity, and in the establishment of immunological tolerance.

Anti-BCR complex antibodies have therapeutic use in the treatment of autoimmunity, cancer, inflammatory disease, and transplantation.

Thus, and as shown herein, PCLX-001 inhibits the BCR, and thus may be used an anti-inflammatory agent, and/or may be used as an anti-autoimmune agent.

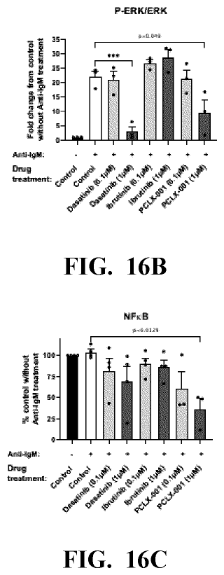

As shown in Supplementary FIGS. 16-18 PCLX-001 inhibits TCR, and thus may be used as an anti-inflammatory agent, and/or may be use as an anti-autoimmune agent. The term “T cell receptor” (TCR) refers to a heterodimer found on the surface of T cells comprising an α chain and a β chain or a γ and a δ chain. T cell receptors recognize processed antigens associated with MHC molecules.

Inhibiting the T Cell receptor (TCR) signal has promise for treating a broad spectrum of human T cell-mediated autoimmune and inflammatory diseases.

In other examples, an NMT inhibitor may inhibit BCR and TCR.

In still other examples, DDD85646 may be used to inhibit BCR and TCR.

In other examples, the NMT inhibitors described in WO 2010/026365, the entire contents of which is hereby incorporated by reference, may be used to inhibit BCR and TCR.

The term “inhibit” or “inhibitor” as used herein, refers to any method or technique which inhibits protein synthesis, levels, activity, or function, as well as methods of inhibiting the induction or stimulation of synthesis, levels, activity, or function of the protein of interest. In some example, the term also refers to any metabolic or regulatory pathway, which can regulate the synthesis, levels, activity, or function of the protein of interest. The term includes binding with other molecules and complex formation. Therefore, the term “inhibitor” refers to an agent or compound, the application of which results in the inhibition of protein function or protein pathway function. However, the term does not imply that each and every one of these functions must be inhibited at the same time.

Accordingly, in some examples, the compounds and compositions herein may be used for treating a subject with, or suspected of having, an inflammatory disorder. In a specific example, PCLX-001 may be used for treating a subject with, or suspected of having, an inflammatory disorder. Thus, in some examples, PCLX-001 may be used as an anti-inflammatory agent.

In other example, DDD85646 may be used for treating a subject with, or suspected of having, an inflammatory disorder. Thus, in some examples, DDD85646 may be used as an anti-inflammatory agent.

In yet other examples, the NMT inhibitors described in WO 2010/026365 may be used for treating a subject with, or suspected of having, an inflammatory disorder. Thus, in some examples, the NMT inhibitors described in WO 2010/026365 may be used as an anti-inflammatory agent.

The term anti-inflammatory refers to the property of a substance or treatment that prevents or reduces inflammation.

As used herein, the terms “disorder” and “disease” are used interchangeably to refer to a condition in a subject. In particular, the term “inflammatory disease” is used interchangeably with the term “inflammatory disorder”.

The term “inflammation”, “inflammatory state” or “inflammatory response” as used herein indicate the complex biological response of vascular tissues of an individual to harmful stimuli, such as pathogens, damaged cells, or irritants, and includes secretion of cytokines and more particularly of pro-inflammatory cytokine, i.e. cytokines which are produced predominantly by activated immune cells such as microglia and are involved in the amplification of inflammatory reactions. In some examples, inflammations include acute inflammation and chronic inflammation.

The term “acute inflammation” as used herein indicates a short-term process characterized by the classic signs of inflammation (swelling, redness, pain, heat, and loss of function) due to the infiltration of the tissues by plasma and leukocytes. An acute inflammation typically occurs as long as the injurious stimulus is present and ceases once the stimulus has been removed, broken down, or walled off by scarring (fibrosis).

The term “chronic inflammation” as used herein indicates a condition characterized by concurrent active inflammation, tissue destruction, and attempts at repair. Chronic inflammation is not characterized by the classic signs of acute inflammation listed above. Instead, chronically inflamed tissue is characterized by the infiltration of mononuclear immune cells (monocytes, macrophages, lymphocytes, and plasma cells), tissue destruction, and attempts at healing, which include angiogenesis and fibrosis.

In some example, the inflammatory disorder is acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

In some examples, the inflammatory disorder is from gastrointestinal disorders (such as peptic ulcers, regional enteritis, diverticulitis, gastrointestinal bleeding, eosinophilic) gastrointestinal disorders (such as, eosinophilic esophagitis, eosinophilic gastritis, eosinophilic gastroenteritis, eosinophilic colitis), gastritis, diarrhea, gastroesophageal reflux disease (GORD, or GERD), inflammatory bowel disease (IBD) (such as Crohn's disease, ulcerative colitis, collagenous colitis, lymphocytic colitis, ischaemic colitis, diversion colitis, Behcet's syndrome, indeterminate colitis) and inflammatory bowel syndrome (IBS)).

In some example, the inflammatory disorder is a disorder of the lung selected from pleurisy, alveolitis, vasculitis, pneumonia, chronic bronchitis, bronchiectasis, diffuse panbronchiolitis, hypersensitivity pneumonitis, asthma, idiopathic pulmonary fibrosis (IPF), and cystic fibrosis.

Accordingly, in some examples, the compounds and compositions herein may be used for treating a subject with, or suspected of having, an autoimmune disease or disorder. In a specific example, PCLX-001 may be used for treating a subject with, or suspected of having, an autoimmune disease or disorder. Thus, in some examples, PCLX-001 may be used as an anti-autoimmune agent.

In other examples, DDD85646 may be used for treating a subject with, or suspected of having, an autoimmune disorder. Thus, in some examples, DDD85646 may be used as an anti-autoimmune agent.

In yet other examples, the NMT inhibitors described in WO 2010/026365 may be used for treating a subject with, or suspected of having, an autoimmune disorder. Thus, in some examples, the NMT inhibitors described in WO 2010/026365 may be used as an anti-autoimmune agent.

As used herein, the terms “disorder” and “disease” are used interchangeably to refer to a condition in a subject. In particular, the term “autoimmune disease” is used interchangeably with the term “autoimmune disorder”.

As used herein, the term “autoimmune disease” refers to any disease state or condition associated with the formation of autoantibodies reactive with the patient's own cells to form antigen-antibody complexes. The term “autoimmune disease” includes conditions which are not normally triggered by a specific external agent, including but not limited to, systemic lupus erythematosus, rheumatoid arthritis, autoimmune thyroiditis and autoimmune hemolytic anemia, as well as those disorders which are triggered by a specific external agent, e.g., acute rheumatic fever.

Other examples of autoimmune disease include, but are not limited to, rheumatoid arthritis, asthma, multiple sclerosis, myasthenia gravis, lupus erythematosus, and insulin-dependent diabetes (type 1) are believed to be examples of autoimmune conditions.

Additional example of autoimmune disease include, but are not limited to, gastritis, colitis, and insulin-dependent autoimmune diabetes, graft transplant/inhibition of rejection, graft vs host disease.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal.

In a specific example, the subject is a human.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early cancer, for example an early stage lymphoma, can be treated to prevent progression or alternatively a subject in remission can be treated with a compound or composition described herein to prevent recurrence.

The term “prevent” or “prevention” refers to prophylactic or preventative measures that prevent and/or slow the development of a targeted pathologic condition or disorder. Thus, those in need of prevention include those at risk of or susceptible to developing the disorder. In certain embodiments, a disease or disorder is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

In some examples, treatment results in prevention or delay of onset or amelioration of symptoms of a disease in a subject or an attainment of a desired biological outcome.

The term “diagnosis” as used herein, refers to the identification of a molecular and/or pathological state, disease or condition, such as the identification of lymphoma, or other type of cancer.

The term “alleviates” as used herein refers to a decrease, reduction or elimination of a condition, disease, disorder, or phenotype, including an abnormality or symptom.

In some example, a pharmaceutically effective amount of PCLX-001 is used. In some examples, a therapeutically effective amount of PCLX-001 is used.

The term “pharmaceutically effective amount” or “effective amount” as used herein refers to the amount of a drug or pharmaceutical agent that will elicit the biological or medical response of a tissue, system, animal or human that is being sought by a researcher or clinician. This amount can be a “therapeutically effective amount”. These terms refer to the amount of a compound and/or compositions described herein which treats, upon single or multiple dose administration, a subject with a disease or condition. An effective amount can be readily determined by the attending diagnostician, as one skilled in the art, by the use of known techniques and by observing results obtained under analogous circumstances. In determining the effective amount, the dose, a number of factors are considered by the attending diagnostician, including, but not limited to: the species of the subject; its size, age, and general health; the specific condition, disorder, or disease involved; the degree of or involvement or the severity of the condition, disorder, or disease, the response of the individual subject; the particular compound administered; the mode of administration; the bioavailability characteristics of the preparation administered; the dose regimen selected; the use of concomitant medication; and other relevant circumstances.

Thus, the term “therapeutically effective amount”, as used herein, refers to an amount effective, at dosages and for periods of time necessary to achieve the desired result. Effective amounts may vary according to factors such as the disease state, age, sex and/or weight of the subject. The amount of a given compound or composition that will correspond to such an amount will vary depending upon various factors, such as the given drug or compound, the pharmaceutical formulation, the route of administration, the identity of the subject being treated, and the like, but can nevertheless be routinely determined by one skilled in the art.

The term “pharmaceutically acceptable” as used herein includes compounds, materials, compositions, and/or dosage forms (such as unit dosages) which are suitable for use in contact with the tissues of a subject without excessive toxicity, irritation, allergic response, or other problem or complication, commensurate with a reasonable benefit/risk ratio. Each carrier, excipient, etc. is also be “acceptable” in the sense of being compatible with the other ingredients of the formulation.

The term “excipient” means a pharmacologically inactive component such as a diluent, lubricant, surfactant, carrier, or the like. Excipients that are useful in preparing a pharmaceutical composition are generally safe, non-toxic and are acceptable for human pharmaceutical use. Reference to an excipient includes both one and more than one such excipient.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), stabilizers and preservatives, and the like.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrastemal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain die active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase maybe a vegetable oil, for example, olive oil or arachis oil, or a mineral oil, for example, liquid paraffin, or it may be a mixtures of these oils.

Suitable emulsifying agents maybe naturally-occurring gums, for example, gum acacia or gum tragacanth; naturally-occurring phosphatides, for example, soybean, lecithin; or esters Or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents, and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

In some examples, treatment methods comprise administering to a subject a therapeutically effective amount of a compound or composition described herein and optionally consists of a single administration or application, or alternatively comprises a series of administrations or applications.

In some examples, formulation(s) may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intratumoral, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

As used herein, the terms “contacting” refers to a process by which, for example, a compound may be delivered to a cell. The compound may be administered in a number of ways, including, but not limited to, direct introduction into a cell (i.e., intracellularly) and/or extracellular introduction into a cavity, interstitial space, or into the circulation of the organism.

Thus, in some example, contacting occurs in vivo. In other examples, contacting may occur in vitro.

A “cell” refers to an individual cell or cell culture. In one example, the cell is a cell obtained or derived from a subject. The culturing of cells and suitable culture media are known.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection.

The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

The compounds and/or compositions described herein may be administered either simultaneously (or substantially simultaneously) or sequentially, dependent upon the condition to be treated, and may be administered in combination with other treatment(s). The other treatment(s), may be administered either simultaneously (or substantially simultaneously) or sequentially.

A “treatment or dosage regimen” as used herein refers to a combination of dosage, frequency of administration, or duration of treatment, with or without addition of a second medication.

A compound or composition may be administered alone or in combination with other treatments, either simultaneously or sequentially, dependent upon the condition to be treated.

In treating a subject, a therapeutically effective amount may be administered to the subject.

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

Compounds and/or compositions comprising compounds disclosed herein may be used in the methods described herein in combination with standard treatment regimes, as would be known to the skilled worker.

In some examples, therapeutic formulations comprising the compounds or compositions as described herein may be prepared for by mixing compounds or compositions having the desired degree of purity with optional physiologically acceptable carriers, excipients or stabilizers, in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 16 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as Tween™ Pluronics™ or polyethylene glycol (PEG).

The therapeutic formulation may also contain more than one active compound as necessary for the particular indication being treated, typically those with complementary activities that do not adversely affect each other. Such molecules are suitably present in combination in amounts that are effective for the purpose intended.

A skilled worked will be able to determine the appropriate dose for the individual subject by following the instructions on the label. Preparation and dosing schedules for commercially available second therapeutic and other compounds administered in combination with or concomitantly with compounds or compositions described herein may be used according to manufacturers' instructions or determined empirically by the skilled practitioner.

Factors which may be taken into account when determining an appropriate dosage include the severity of the disease state, general health of the subject, age, weight, and gender of the subject, diet, time and frequency of administration, the particular components of the combination, reaction sensitivities, and tolerance/response to therapy.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in any way.

### EXAMPLES

### Abstract

Myristoylation, the N-terminal modification of proteins with the fatty acid myristate, is critical for membrane targeting and cell signaling. Because cancer cells often have increased N-myristoyltransferase (NMT) expression, NMTs were proposed as anti-cancer targets. To systematically investigate this, we performed robotic cancer cell line screens and discovered a marked sensitivity of hematological cancer cell lines, including B-cell lymphomas, to the potent pan-NMT inhibitor PCLX-001. PCLX-001 treatment impacts the global myristoylation of lymphoma cell proteins and inhibits early B-cell receptor (BCR) signaling events critical for survival. In addition to abrogating myristoylation of Src family kinases, PCLX-001 also promotes their degradation and, unexpectedly, that of numerous non-myristoylated BCR effectors including c-Myc, NFkB and P-ERK, leading to cancer cell death in vitro and in xenograft models. Because some treated lymphoma patients experience relapse and die, targeting B-cell lymphomas with a NMT inhibitor potentially provides an additional much needed treatment option for lymphoma.

### Results

### PCLX-001 Selectively Kills Blood Cancer Cells In Vitro.

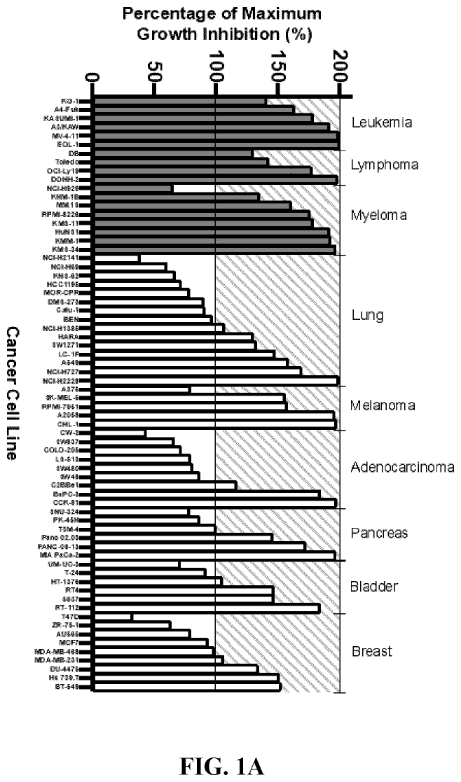

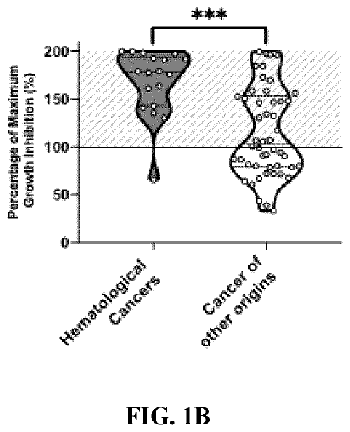

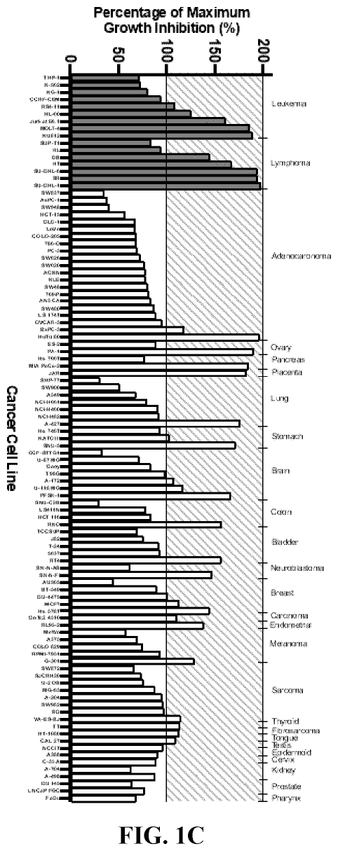

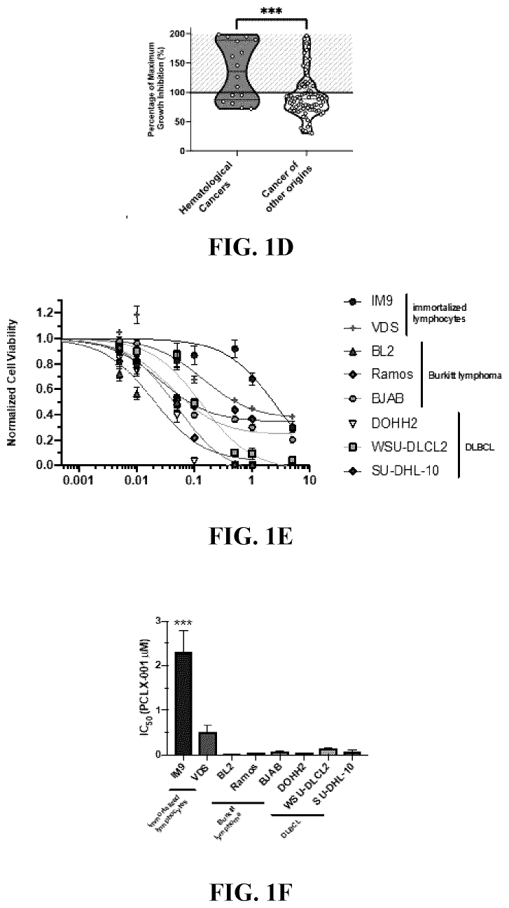

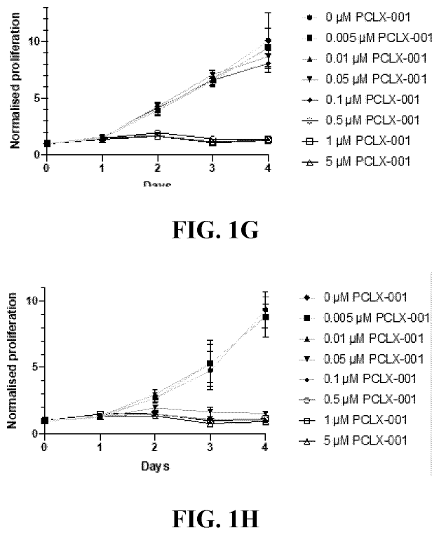

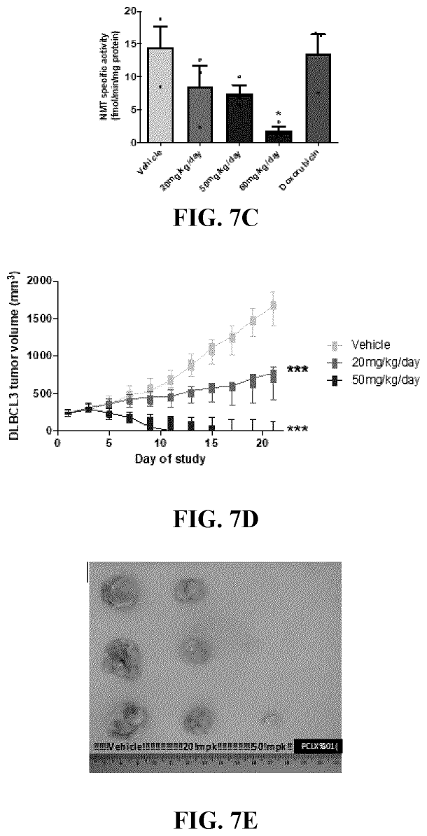

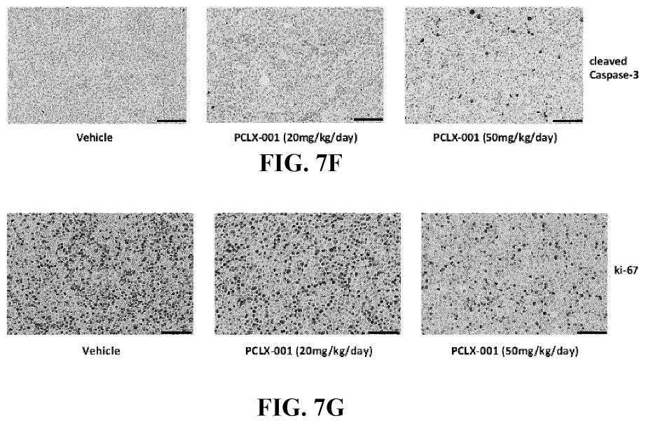

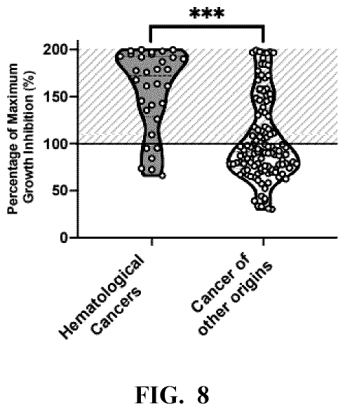

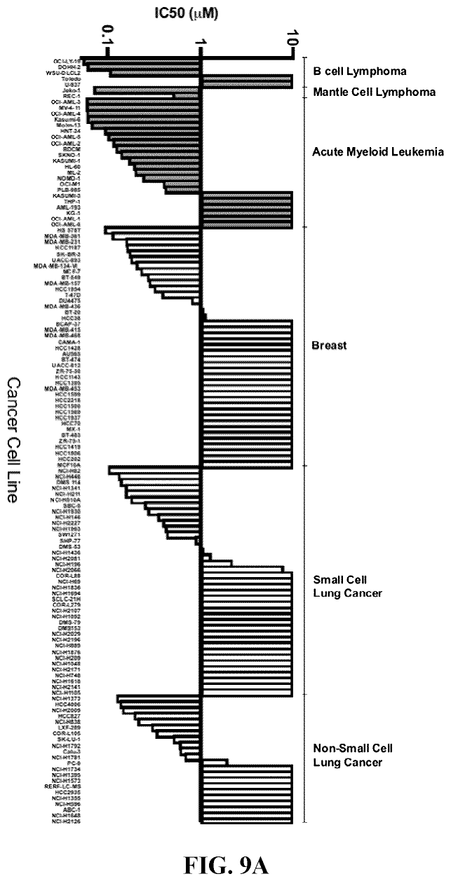

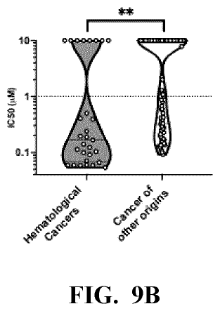

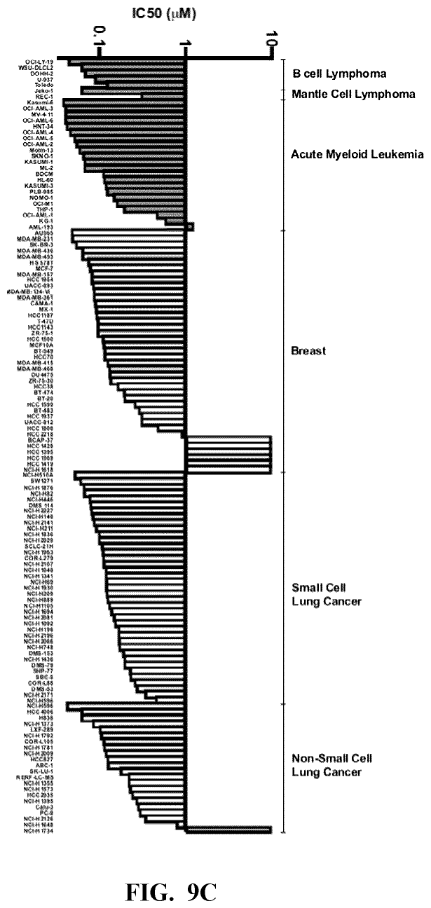

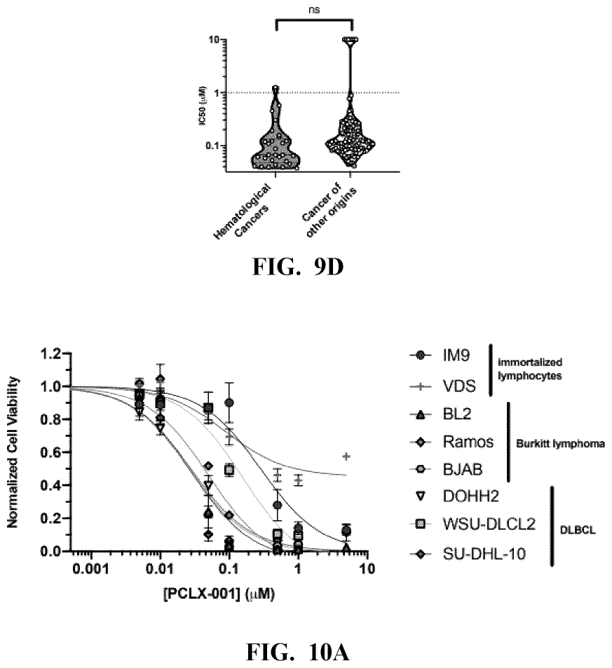

To investigate the therapeutic potential of NMT inhibition in cancer, we performed three independent robotic screens to measure the percentage growth inhibition (GI) of PCLX-001 in a variety of cancer cell lines. Using 68 cell lines on the Horizon (St. Louis, MO) platform, we show PCLX-001 inhibits the growth of a variety of cell lines (FIG. 1A). GI is significantly higher (P<0.0001) however, in hematological (blood) cancer cells including lymphomas, leukaemia, and myelomas than in other cancer cell line types (FIG. 1B). These results were recapitulated using a 161 cell line Oncolines™ (Oss, Netherlands) screen (FIG. 1C, D, P=0.0001, FIG. 7), and in a third screen (Chempartner, Shanghai, China) whereby 131 cancer cell lines were exposed to PCLX-001 for 3 and 6 days (FIG. 9). The median ICs. following 3 days of PCLX-001 treatment is significantly lower in hematological cancer cell lines (0.166 μM) in comparison to cell lines originating from solid tumors (10 μM, the highest dose tested; P=0.0038) including breast cancer, non-small cell lung cancer (NSCLC) and small cell lung cancer (SCLC) (FIG. 9A, B). By day 6 however, PCLX-001 effectively kills nearly all types of cancer cell lines tested (FIG. 9C, D).

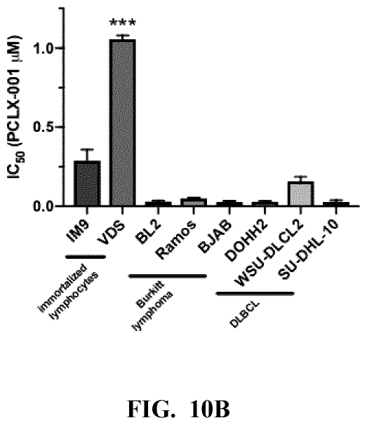

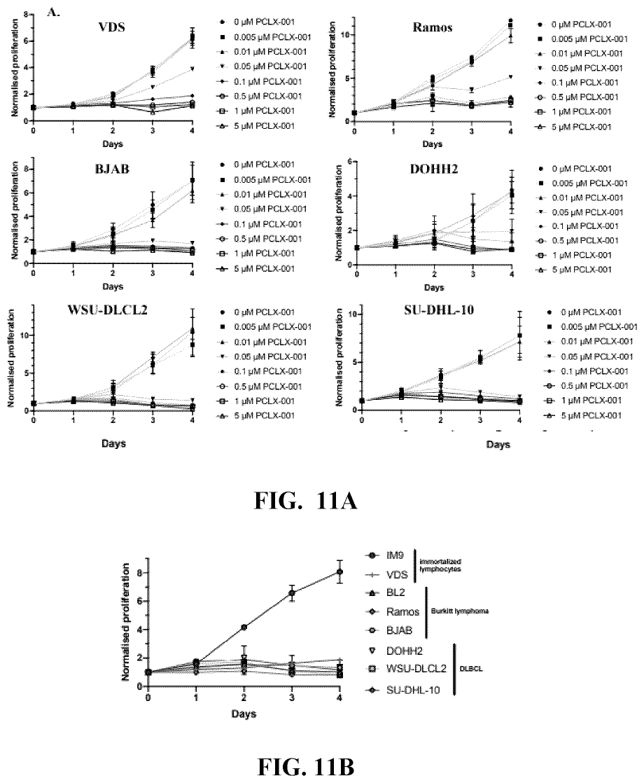

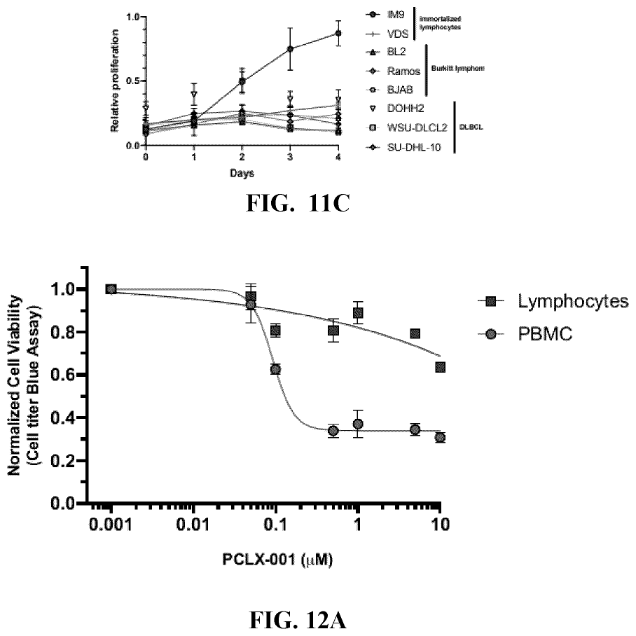

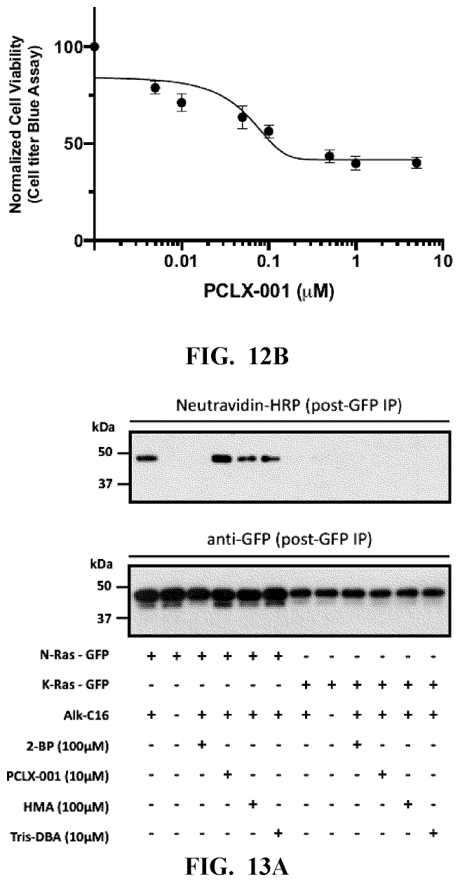

To confirm the data obtained using screens, we tested the effects of PCLX-001 treatment on several common B-cell lymphoma cell lines including the BL cell lines BL2, Ramos, and BJAB, the DLBCL cell lines DOHH2, WSU-DLCL2, and SU-DHL-10, and the immortalized B-cells IM9 and VDS⁴⁶ as controls. We performed three types of assays on these cells: 1) CellTiter Blue assay, whose readouts are dependent on both proliferation (number of total cells) and viability (percentage of viable cells) to evaluate the total number of viable cells, 2) Calcein assay, whose readout is independent of proliferation rate as it only measures the percentage of viable cells and, 3) a cell proliferation assay to simply count the total number of cells over time, independently of their viability. Incubation of malignant cell lines with PCLX-001 kills these cells in a time and concentration dependent manner in all three assays. Furthermore, PCLX-001 treatment kills malignant cell lines at significantly lower concentrations than that needed to kill benign IM9 and VDS B-cells as measured by both CellTiter Blue (4 to 111 fold less PCLX-001 needed; FIG. 1E, F) and Calcein (2 to 40 fold less PCLX-001 needed; FIG. 10) assays. PCLX-001 is also better at inhibiting the proliferation and viability of the 6 malignant B-lymphoma cell lines in comparison to benign IM9 and VDS B-cells (FIG. 1 E-H, FIGS. 10 and 11). To illustrate this, we show the treatment of malignant BL2 cells with 0.05 and 0.1 μM PCLX-001 completely inhibits their proliferation over time with little effect on benign IM9 cells at these concentrations (FIG. 1G, H). Importantly, 96 hr PCLX-001 treatment of freshly isolated human lymphocytes and peripheral blood mononuclear cells (PBMCs) only marginally affects lymphocyte survival, whereas 0.1 μM PCLX-001 causes an ˜50% decrease in PBMC survival (FIG. 12). The surviving PBMCs however, endure PCLX-001 treatment up to a concentration of 10 μM, a dose ˜100× greater than the IC₅₀ (˜0.050-0.100 μM) for most hematological cancer cell lines in vitro. A similar trend is observed in primary human umbilical vein endothelial cells (HUVECs; FIG. 12B). Taken together, these data show that PCLX-001 treatment selectively inhibits the proliferation and viability of a variety of cancer cell lines in a time and concentration dependent manner, and is particularly efficient at killing malignant hematologic cancer cells in vitro.

### Myristoylation Inhibition Induces Lymphoma Cell Apoptosis

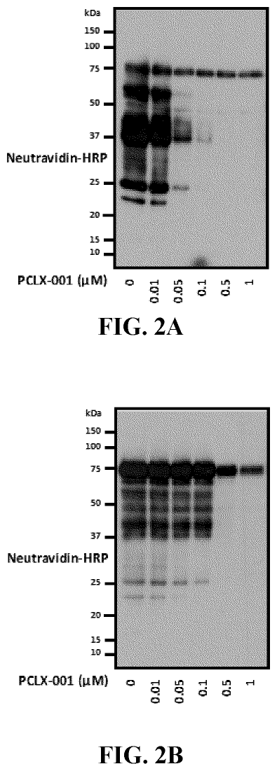

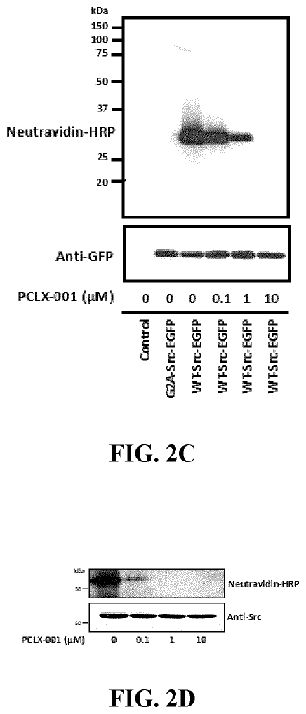

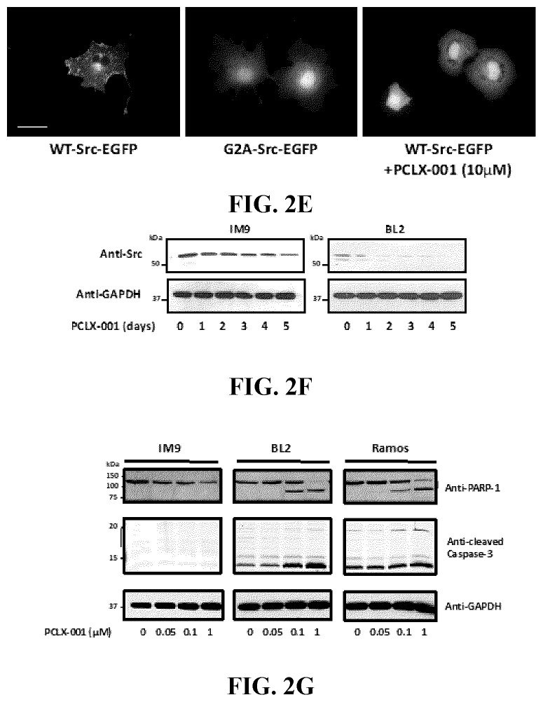

To verify that PCLX-001 acts on target, we used click chemistry as described⁴⁷ to visualize the inhibition of endogenous protein myristoylation in malignant BL2 lymphoma cells and benign IM9 B-cells (FIG. 2A, B). PCLX-001 inhibits total protein myristoylation in a concentration dependent manner in both cell lines. However, only ˜0.1 μM of PCLX-001 is required to decrease BL2 myristoylation compared to 5 times this amount in IM9 cells (FIG. 2A, B). This suggests that protein myristoylation processes in malignant BL2 cells may somehow be more sensitive to PCLX-001 inhibition. Although PCLX-001 (Table 1)³⁸ is a closely related analog of DDD85646/IMP-366 and part of s series of recently validated NMT inhibitors^{38, 39}, we further evaluated its effect on palmitoylation and phosphorylation. PCLX-001 does not inhibit the palmitoylation of an EGFP-N-Ras construct expressed in COS-7 cells (FIG. 13A), nor does it significantly inhibit any of the 468 human kinases of the pre-configured scanMAX KINOMEscan™ (Eurofins DiscoverX, San Diego, USA) at concentrations up to 10 μM (FIG. 13B). Of note, only 3 possible positive hits were found at 196 μM PCLX-001, a concentration ˜4000 times greater than the EC50 of PCLX-001 for BL2 cells. Thus, the time and concentration dependent effects of PCLX-001 on cellular function and viability appear NMT-specific.

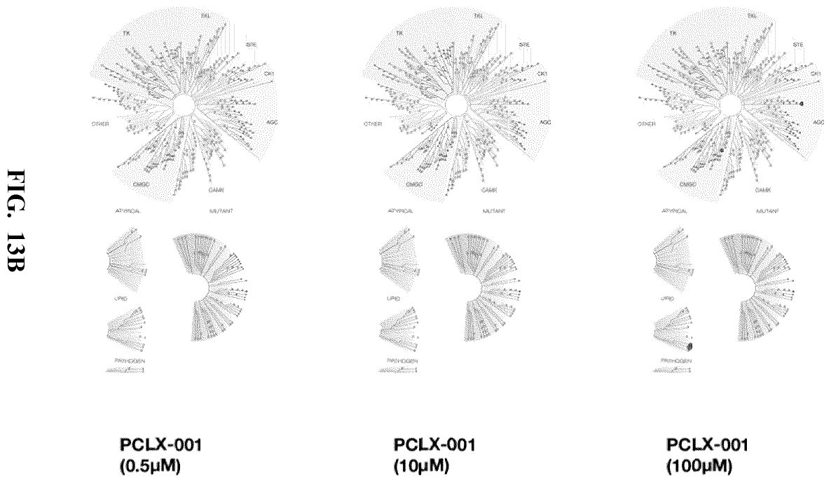

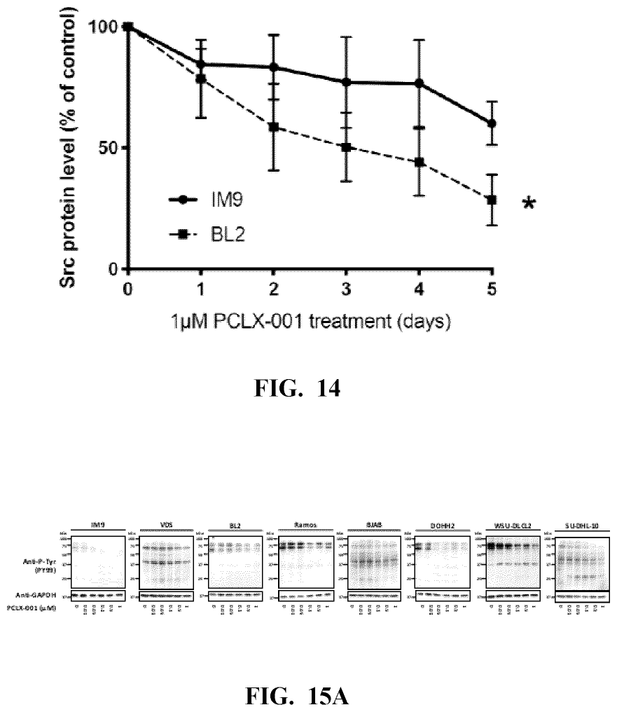

We next verified PCLX-001 inhibition of NMT function by monitoring the myristoylation and localization of Src protein tyrosine kinase, a known myristoylated protein, using truncated Src-EGFP⁴⁸ constructs expressed in COS-7 cells by click chemistry⁴⁷ and fluorescence microscopy. PCLX-001 inhibits the myristoylation of both the WT-Src-EGFP construct and endogenous Src in a concentration dependent manner in COS-7 and IM9 cells, respectively (FIG. 2C, D). Notably, myristoylation inhibition relocalizes WT-Src-EGFP from the plasma and endosomal membranes to the cytoplasm in COS-7 cells, producing a distribution pattern comparable to that of the non-myristoylatable Gly2Ala-Src-EGFP mutant construct⁴⁸ (FIG. 2E). Inhibiting endogenous Src myristoylation also produces an unexpected time-dependent reduction in Src protein levels in BL2 and IM9 cells treated with PCLX-001 for up to 5 days (FIG. 2F) that is accelerated in malignant BL2 cells in comparison to IM9 controls (P=6.6174; FIG. 14). Furthermore, PCLX-001 treatment selectively induces apoptosis in the BL cell lines BL2 and Ramos, but not immortalized IM9 B-cells as measured by PARP-1 and caspase-3 cleavage (FIG. 2G), consistent with benign, immortalized B-cells exhibiting a higher threshold for PCLX-001 toxicity (FIG. 1E, F, FIG. 10). Altogether, these data suggest that PCLX-001 preferentially abrogates myristoylation in malignant lymphoma cells in comparison to normal immortalized B cells leading to selective cell death.

### PCLX-001 Reduces SFK Levels and BCR Downstream Signaling

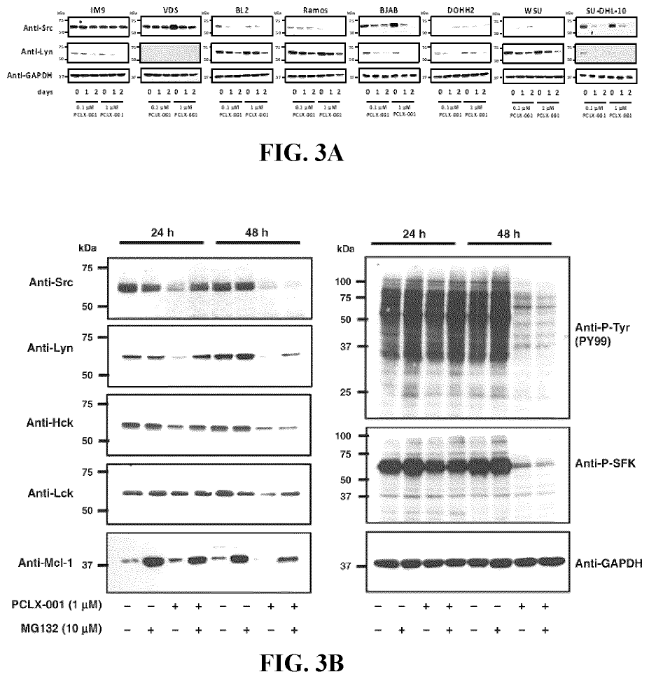

BCR signaling provides key survival signals in B-cell lymphomas, and SFKs (especially Lyn) play a critical role in initiating BCR signaling in both normal B-cells and lymphomas^{5, 6, 11, 49, 50}. Since PCLX-001 treatment preferentially reduces endogenous Src protein levels in malignant BL2 cells in comparison to benign IM9 controls (FIG. 2F), we sought to determine if a similar effect could be observed on other SFKs in various lymphoma cell lines. We found that PCLX-001 treated BL2, Ramos, BJAB, DOHH2, WSU-DLCL2 and SU-DHL-10 lymphoma cells all exhibit a more pronounced dose and time dependent decrease in Src and Lyn SFK protein levels in comparison to benign IM9 and VDS controls (FIG. 3A). To investigate whether the proteasome degradation mechanism was involved, after addition of PCLX-001 to BL2 cells for 24 or 48 hours, we treated BL2 cells with the proteasome inhibitor MG132 or not for 6 hrs prior to harvesting and lysing the cells, and, measuring residual protein levels of not only Src and Lyn SFKs, but also hematopoietic cell kinase (Hck) and lymphocyte specific kinase (Lck) SFKs, both of which are also linked to lymphoma progression. PCLX-001 treatment reduces Hck and Lck protein levels to a lesser degree than Src and Lyn (FIG. 3B). However, the addition of MG132 to PCLX-001 treated cells results in partial or complete restoration of the 4 SFK proteins in comparison to controls, especially at the 24 h time point (FIG. 3B). This indicates that the degradation of non-myristoylated-SFKs can be attributed in part to the ubiquitin-proteasome system. The efficacy of the proteasome inhibition by MG132 was confirmed by monitoring Mcl-1 levels, a protein actively degraded by the proteasome⁵¹ (FIG. 3B).

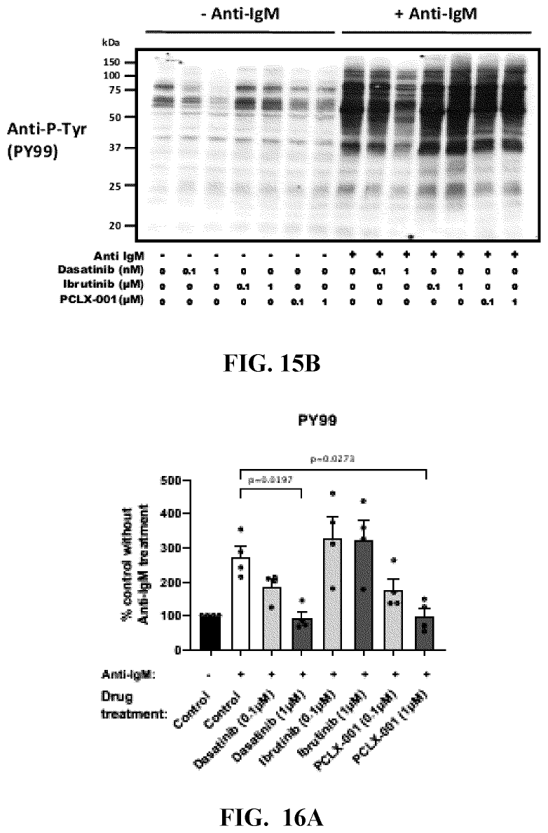

Because antigen independent basal BCR signaling is often elevated in lymphoma cells^{6, 49}, we assessed the impact of PCLX-001 treatment on ligand independent BCR signaling by monitoring endogenous tyrosine phosphorylation levels in the above cell lines using an anti-phospho-tyrosine (P-Tyr) antibody (PY99). 24 hr treatments with PCLX-001 decreases antigen independent global phospho-tyrosine levels in all cell lines tested in a concentration dependent manner (FIG. 15A). In addition, 1 μM PCLX-001 abrogates nearly all ligand dependent BCR mediated phospho-tyrosine and pan-phospho-SFK levels in BL2 cells after BCR ligation with anti-IgM (FIG. 3B). While proteasome inhibition results in the stabilization of SFKs as suggested by their increased protein levels, it does not reverse the impact of PCLX-001 on ligand independent tyrosine phosphorylation, or overall SFK phosphorylation in BL2 cells (FIG. 3B) supporting the established notion that non-myristoylated SFKs are no longer functional because of their mislocalization and their inability to phosphorylate their substrates. Altogether, these results indicate that the myristoylation of SFKs is essential for both their activity and stability, and is required for downstream BCR signaling in lymphoma cells.

### PCLX-001 Potently Inhibits BCR Survival Signaling Components

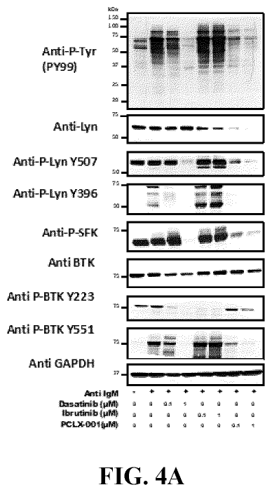

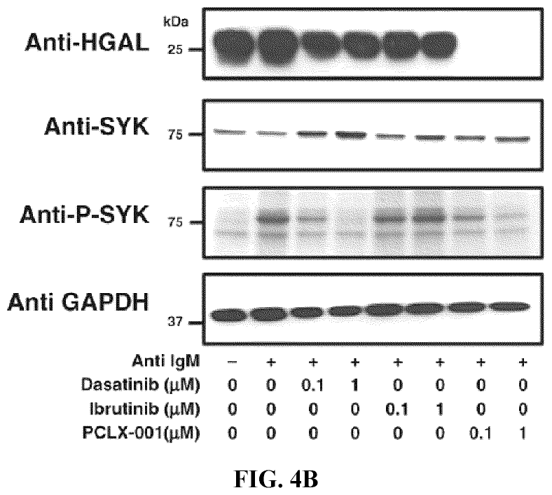

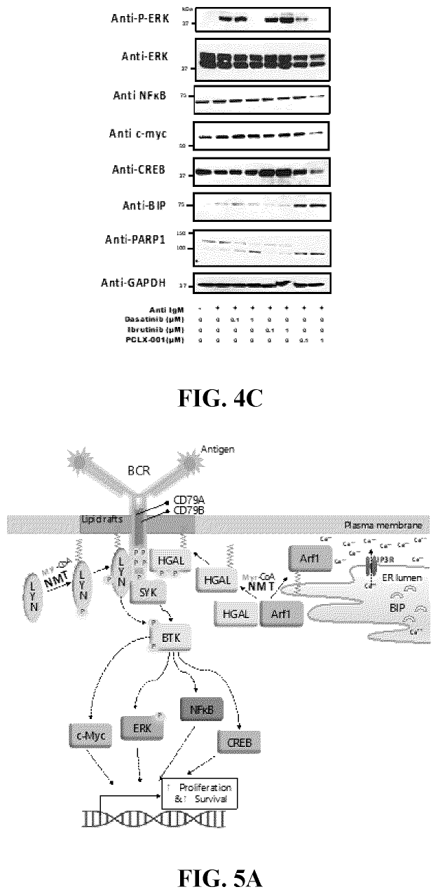

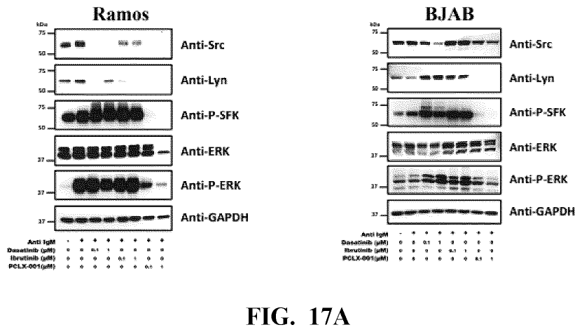

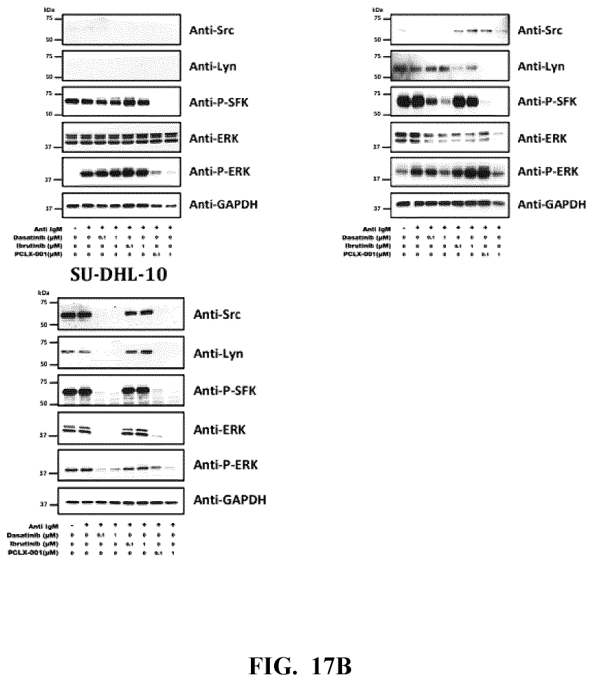

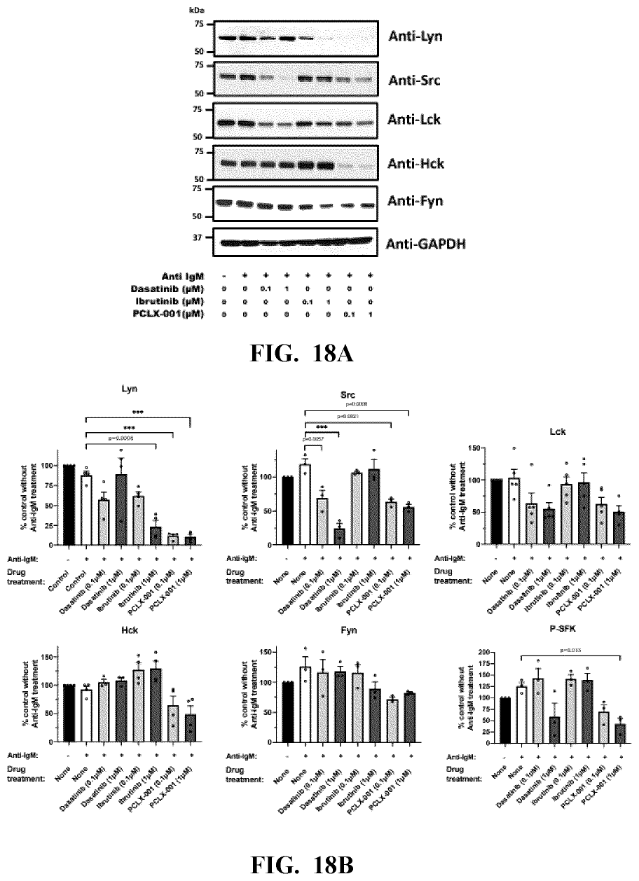

Since PCLX-001 impacts SFK protein levels and ligand dependent BCR mediated tyrosine phosphorylation, we next evaluated its effects on other BCR mediated signaling intermediates using two clinically approved BCR signaling inhibitors: dasatinib (a broad spectrum tyrosine kinase inhibitor) and ibrutinib (a BTK inhibitor) as controls⁵². Because BL2 cells were found to be most responsive to anti-human IgM BCR stimulation (FIG. 15B), these cells were chosen as a model for studying PCLX-001-mediated effects on activated BCR signaling. BL2 cells treated with 0.1 or 1.0 μM PCLX-001 exhibit concentration dependent partial (at 24 hrs, FIG. 15B) and near complete abrogation (at 48 hrs) of anti-IgM stimulated BCR mediated tyrosine phosphorylation (FIG. 4A, quantification in FIG. 16). The overall reduction in tyrosine phosphorylation is more pronounced in BL2 cells treated with PCLX-001 than those treated with dasatinib or ibrutinib at the same concentrations. PCLX-001 treatment also reduces or abolishes levels of total Lyn, activated-phosphorylated-Lyn (Y396), as well as that of total BTK and activated-phosphorylated-BTK (Y223) in BL2 cells (FIG. 4A). These findings were confirmed in several other lymphoma cell lines (FIG. 17) and for several other SFKs including Src, Lck, Hck, and Fyn, as well as for activated-pan-phospho-SFKs in BL2 cells (FIG. 4A, FIG. 18). Dasatinib and ibrutinib selectively inhibited their respective targets as measured using anti-P-Lyn, anti-P-SFKs and anti-P-BTK antibodies (FIG. 4A).

PCLX-001 treatment also mediates the reduction of other myristoylated protein levels including the BCR signaling enhancer protein HGAL and Arf1 GTPases while dasatinib and ibrutinib have no effect on the levels of either of these proteins (FIG. 4B,C). Of note, the loss of HGAL protein was much faster than that of SFKs and Arf1 GTPase and the loss of HGAL protein levels is associated with a reduction in the phosphorylated and active form of SYK as expected^{14, 15} (FIG. 4B). Since the levels of both myristoylated HGAL and myristoylated small GTPase Arf1 are also diminished upon PCLX-001 treatment, the ability of PCLX-001 to promote the degradation of myristoylated proteins is therefore not restricted to myristoylated SFKs (FIG. 4).

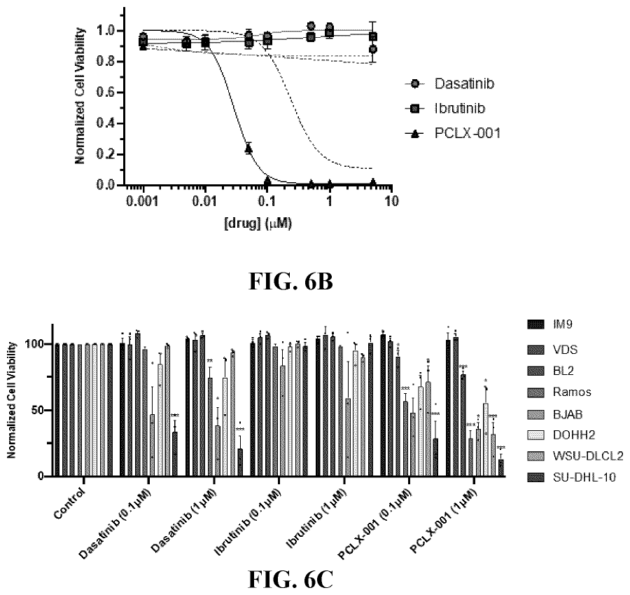

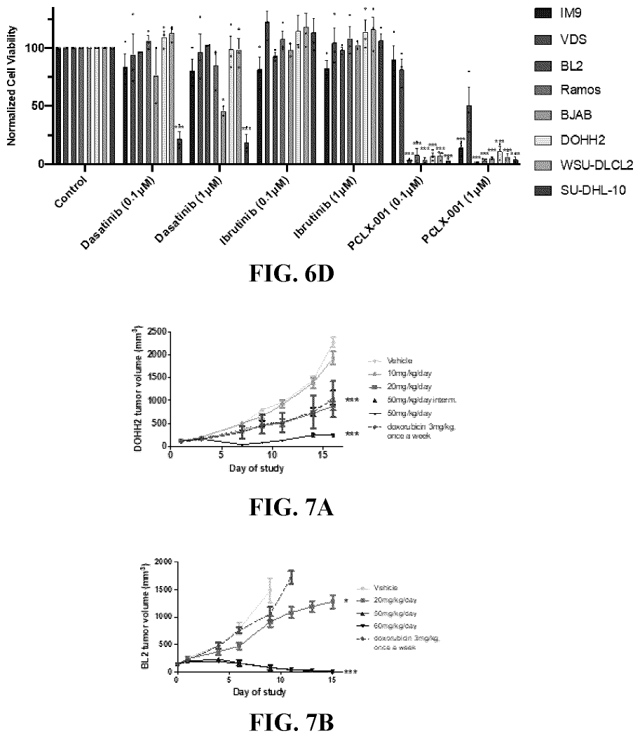

BCR signaling ultimately converges on transcription factors involved in B-cell proliferation and survival including phospho-ERK (P-ERK), NFκB, c-Myc and CREB^{4, 5}. Thus, we evaluated the effects of PCLX-001, dasatinib and ibrutinib on these effectors at 0.1 and 1.0 μM for 48 hours on BL2 cells. Of note, these treatments resulted in less than 25% cell death for PCLX-001 at 48 hours and less than 5% for dasatinib and ibrutinib at either concentrations used (FIGS. 6A and 6C). Consistent with an impairment in BCR signaling, PCLX-001 reduces the levels of P-ERK, NFκB, c-Myc and CREB in a concentration dependent manner with statistically significant decreases (P<0.05) detected in phospho-ERK and NFκB levels (FIG. 4C, quantification in FIG. 16). Again, these effects tend to be more marked in PCLX-001 treated cells than those treated with either dasatinib or ibrutinib. These findings, including decreased levels of Src, Lyn, pan-P-SFK, ERK and P-ERK, are also observed in several other malignant lymphoma cell lines (FIG. 17). We also show PCLX-001 treatment increased the levels of the ER stress pro-apoptotic marker Bip more than dasatinib and ibrutinib treatments leading to an overall increase apoptosis as measured by caspase-cleaved PARP1 (FIG. 4C). Therefore, the ability of PCLX-001 to promote the degradation of proteins is not restricted to its effects on myristoylated proteins such as SFKs, HGAL and Arf1 but also includes effects on non-myristoylated proteins such as phospho-ERK and NFκB signaling downstream the BCR.

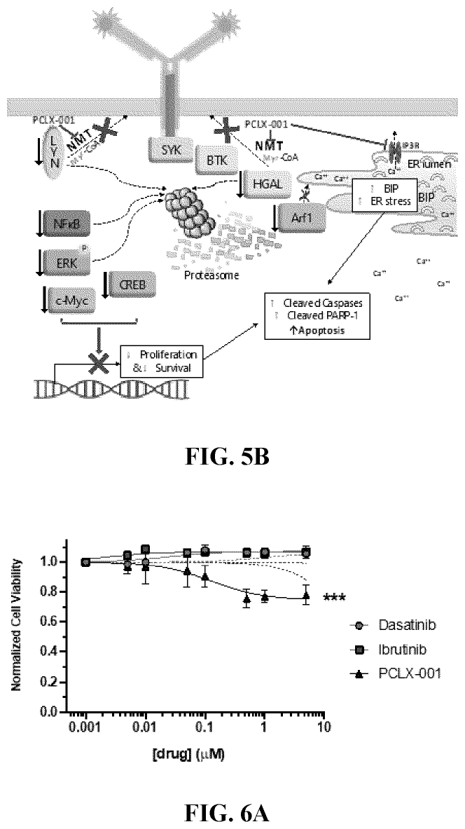

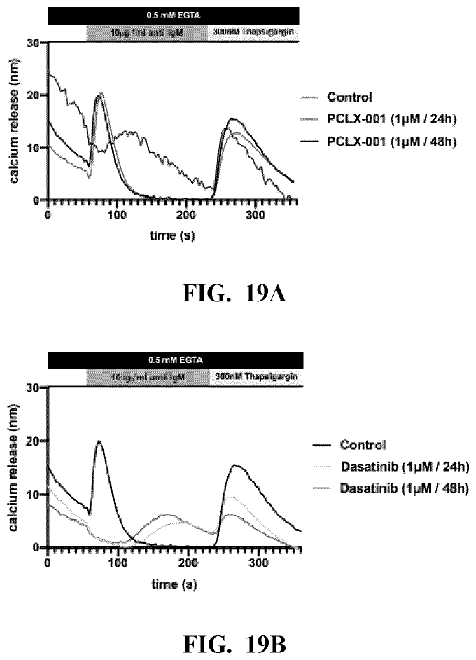

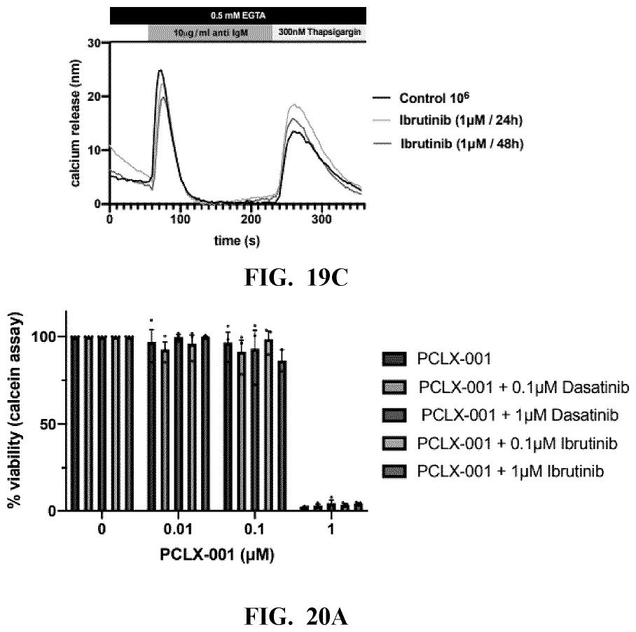

Early events in BCR signaling also culminate in the activation of phospholipase Cr and calcium mobilization in the cytosol. We demonstrate that PCLX-001 (1 μM) treatment of BL2 cells for 48 hours potently inhibits anti-IgM BCR-induced calcium mobilization from intracellular stores using a fluorescent ratiometric Fura-2 Ca⁺⁺-chelator assay⁵³ (FIG. 19). In addition to drastically reducing the intensity of the calcium release peak, and similarly to dasatinib treatment, PCLX-001 delayed the calcium release process. Overall, PCLX-001 inhibited calcium mobilization more than either dasatinib and ibrutinib used at the same concentration. Of note, extended treatment of BL2 cells with PCLX-001 for 48 hours interfered with calcium homeostasis and lead to increased basal levels of cytosolic calcium (FIG. 19), perhaps contributing to ER calcium depletion and apoptosis. In all, our data indicate that PCLX-001 treatment effectively impairs BCR-mediated pro-survival signaling and induces apoptosis in lymphoma cells (FIG. 5).

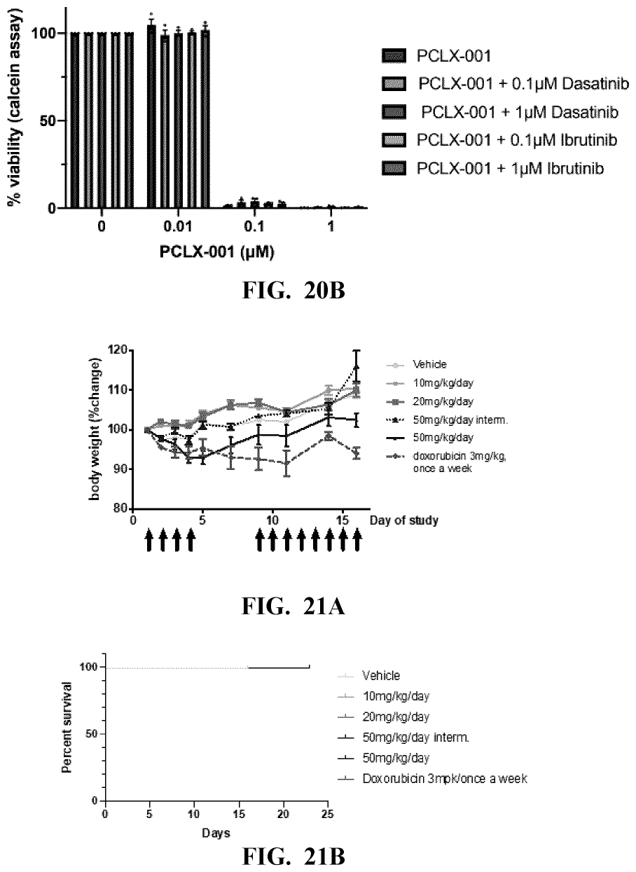

Because PCLX-001, dasatinib and ibrutinib varied in potency and differentially affected downstream BCR signaling, we next compared the effects of these drugs on the overall viability of the lymphoma cell lines tested above. Dasatinib and ibrutinib treatments have minimal effect on BL2 (solid lines) and IM9 (dotted lines) cells following 48 and 96 hrs of treatment, whereas PCLX-001 kills malignant BL2 cells (solid line) at a substantially lower concentration than that required to kill benign, IM9 controls (dotted line) (FIG. 6A, B). Similar trends in cell viability are observed across all other cell lines with exception of SU-DHL-10, which was equally sensitive to both PCLX-001 and dasatinib (FIG. 6C, D). Importantly, the combination treatment of either dasatinib or ibrutinib at concentrations of 0.1 and 1.0 μM to PCLX-001 at 0.01, 0.1 and 1.0 μM does not further decrease viability suggesting that PCLX-001 effects are mediated upstream of dasatinib and ibrutinib targets (FIG. 20). Altogether, PCLX-001 has the broadest spectrum of potency against malignant lymphoma cell lines at both 48 and 96 hrs in comparison to dasatinib and ibrutinib, and is better at sparing benign, immortalized IM9 and VDS B-cell controls, demonstrating higher selectivity and an in vitro therapeutic window superior to that of two clinically approved drugs.

### NMT Expression is Altered in Hematologic Cancer Cells

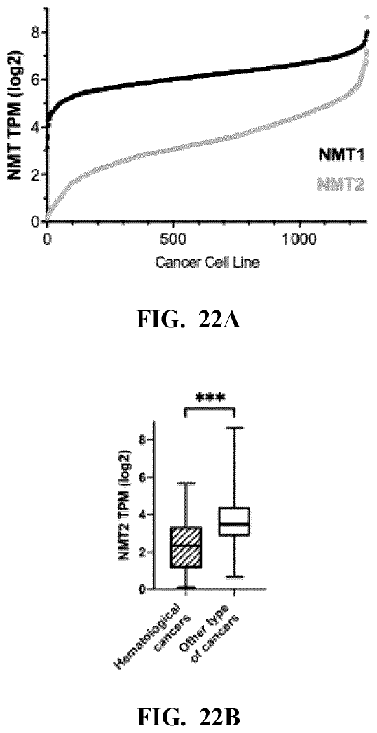

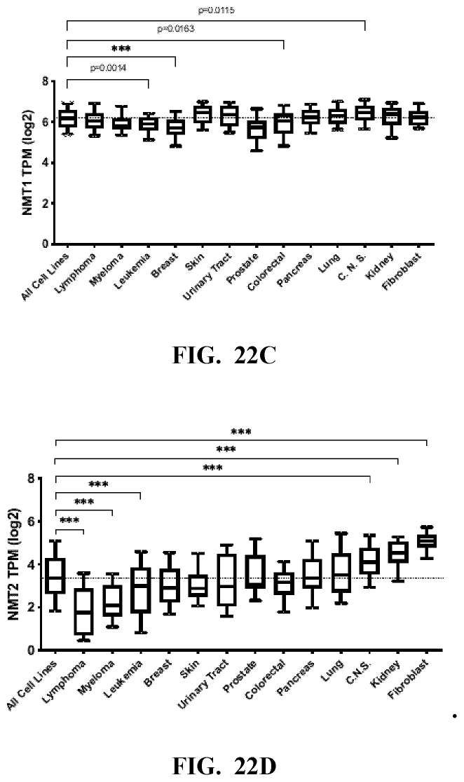

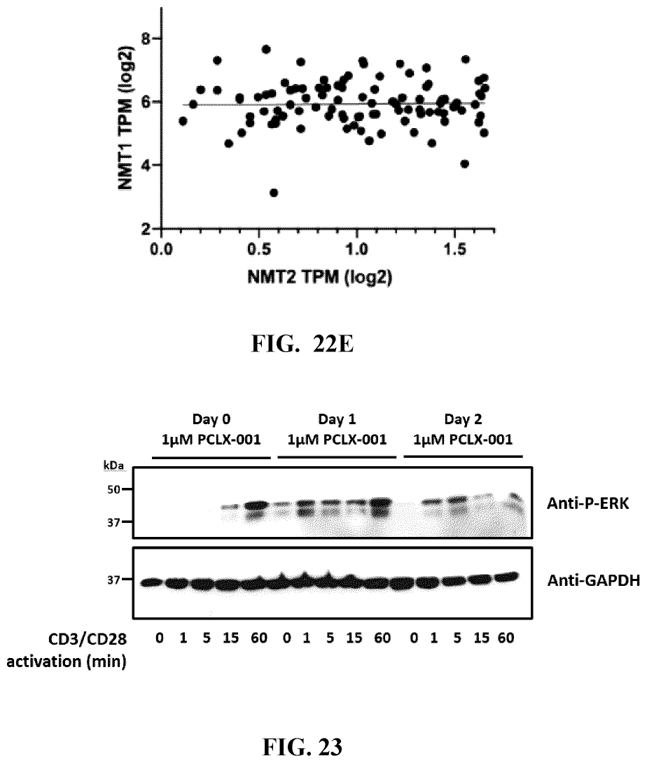

While we still do not know why hematological cancer cells are more vulnerable to PCLX-001 than other cancer cell types, we think this might be related to alterations in NAMT or NMT2 expression in hematological cancer cells. To substantiate this possibility, we performed in silico analyses of gene expression data from the Cancer Cell Line Encyclopedia⁵⁴. We first find that the NMTJ number of transcripts is about eight times (2³) the number of NMT2 transcripts in all cell lines on average, and second, that there is a heterogenous but significant reduction of NMT2 expression in numerous hematological cancer cell lines in comparison to other types of cancer cell lines (FIG. 22A,B). Expression of NMT1 is relatively constant across the 1269 cell lines investigated with a slight but significant decrease in expression in breast and leukemia cancer cell lines while NMT2 expression varies significantly amongst various cancers and also within a given cancer type (FIG. 22C,D). The data also illustrate that while the expression of NMT2 is higher in cancer cell lines of CNS, kidney and fibroblast origins there is a selective and significant reduction of NMT2 expression in hematological cancers such as leukemia, lymphoma and myeloma (FIG. 22D). Interestingly, the low NMT2 expression levels seen in lymphomas, leukemia and other cell lines were not compensated by an increase in NMT1 expression (FIG. 22E). Altogether, we find a reduction in NMT2 expression in hematological cancer cell lines, which may account for their increased sensitivity to PCLX-001.

### PCLX-001 Treatment has Potent Anti-Tumor Activity In Vivo

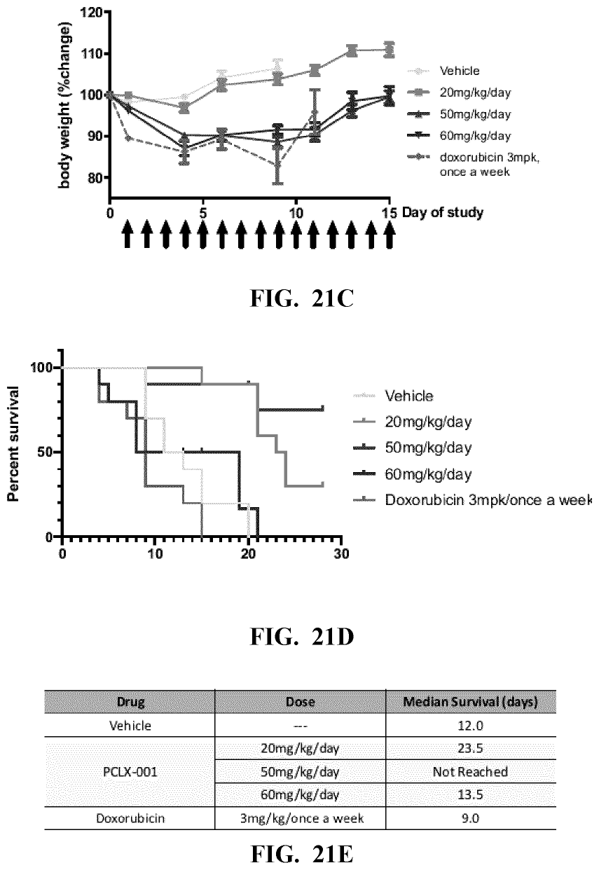

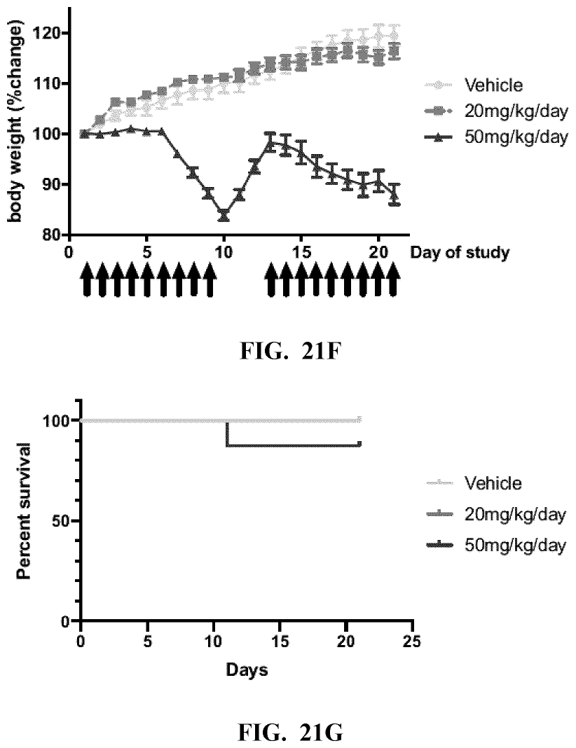

Based on lymphoma cell sensitivity to NMT inhibition in vitro, we investigated whether PCLX-001 could mitigate tumor progression in vivo in two murine lymphoma cell line-derived subcutaneous tumor xenograft models and used doxorubicin as a clinically approved drug reference. In mice bearing DOHH2 tumors, PCLX-001 demonstrates a significant tumoricidal effect when given daily at 20 mg/kg or every other day at 50 mg/kg (P<0.001) (FIG. 7A). At 50 mg/kg daily, PCLX-001 reduces tumor size by up to 70% by day 7 (average tumor size at day 7=44.0±8.1 mm³), but this was accompanied by significant weight loss, necessitating a 5-day treatment interruption (FIG. 21A). Upon resuming treatment, a mean tumor growth inhibition (TGI) of 95% is observed by day 16. By comparison, doxorubicin treatment causes a 57% TGI and reduced body weight by up to 8% (FIG. 21A). Importantly, treatment with PCLX-001 does not increase mortality at any dose (FIG. 21B).

In mice bearing BL2 xenografts, PCLX-001 shows partial TGI at doses of 20 mg/kg daily reaching 42.5% tumor regression by day 9 (P=0.016) (FIG. 7B). Furthermore, 56 or 60 mg/kg daily doses of PCLX-001 cause 100% tumor regression in 9 of 9 and 7 of 7 surviving mice, respectively, when administered for 13 days. Kaplan-Meier survival analysis of this xenograft model also shows that PCLX-001 doses between 20-50 mg/kg/day prolongs the survival of BL2 tumor bearing mice in comparison to untreated, vehicle controls (FIG. 21D, E). Doxorubicin by contrast has no effect on BL2 tumor growth (FIG. 7B), and treatment was terminated at day 11 due to the adverse effects (FIG. 21C). At the conclusion of treatment, we measured NMT activity²¹ in BL2 tumor lysates and find it to be reduced in a PCLX-001 concentration-dependent manner (P=0.03; FIG. 7C) showing that PCLX-001 acts on target in vivo.

Because cell line derived xenografts lack the complexity of human tumors, we dissected and propagated a DLBCL lymphoma derived from patient DLBCL3 whose cancer was refractory to multiple lines of chemotherapy including CHOP, RICE, intrathecal methotrexate/cytarabine, and DHAP (Table 2) to establish a patient-derived xenograft model in NODscid mice. Treatments were assessed in groups of 8 mice each. A 20 mg/kg subcutaneous daily dose of PCLX-001 treatment for 21 days results in 66% TGI (P<0.001; FIG. 7D). This dose was then increased to 50 mg/kg daily in another set of mice for two 9-day periods separated by a 3-day treatment interruption to allow the mice to recover from ˜15% loss of body weight (FIG. 21F). Following this higher dose regimen, PCLX-001 administration results in complete tumor regression in 6 of 7 surviving mice at day 13 (FIG. 7D) with one mouse with no detectable tumors dying at day 11 (FIG. 21G). Surgically removed tumors from vehicle-control and PCLX-001 treated mice confirm a concentration-dependent reduction in overall tumor size following 21 days of PCLX-001 treatment (FIG. 7E) concomitant with increased in apoptosis (increased cleaved caspase-3; FIG. 7F) and reduction in cell proliferation (as determined by Ki-67 analysis; FIG. 7G). Thus, PCLX-001 treatment induces apoptosis and cell-cycle arrest in a patient-derived lymphoma tumor in vivo in a dose-specific manner. The effect of doxorubicin treatment could not be assessed due to severe drug toxicity and death in the majority of tumor bearing mice within the first 7 days of the experiment.

TABLE 2
Description of tumor and patient DLBCL3 used in
murine patient-derived tumor xenograft study.

| | DLBCL3 |
|---|---|
| Age at PDX tissue | [symbol] |
| harvest | |
| Gender | Male |
| Clinical | Previous diagnosis of |
| presentation | DLBCL, now presenting |
| | with [symbol] |
| | and [symbol] involvement |
| Diagnosis | Diffuse large B-cell [symbol] |
| Cell of origin | [symbol] |
| Immunophenotype | [symbol] |
| EBER | Negative |
| Generic | Rearrangements of [symbol] |
| alterations | and [symbol] |

[symbol]

indicates data missing or illegible when filed

### Mice Tolerate PCLX-001 at Efficacious Dose Levels

Mice tolerated PCLX-001 at efficacious doses without specific end-organ toxicity. All mice treated with PCLX-001 survived the first xenograft study (FIG. 7A), while some mice treated with PCLX-001 at higher dose levels died in the other two studies (FIG. 7B,D). Neither the clinical pathology nor anatomic pathology evaluations identified the cause of death. Findings suggesting toxicity were seen in two studies. Of three mice bearing BL2 xenografts and given PCLX-001 at 50 mg/kg daily with a short treatment holiday, all had lower-than-normal neutrophil and lymphocyte counts at the end of the dosing period, and one also had lower-than-normal monocyte and platelet counts. In mice bearing DLBCL lymphocyte xenografts and given PCLX-001 at 20, 50, or 60 mg/kg daily, signs of ill health (e.g. rough and scruffy coats; piloerection) were noted in most mice at all dose levels, and dehydration and weight loss were noted at 50 and 60 mg/kg daily (Tables 3-8).

TABLE 3
Influence of PCLX-001 and doxorubicin treatment on serum chemistry values in DOHH2
NODscid mouse xenograft model (Supplementary Note 1). Measurements were averaged
by treatment group (n = 3). Standard error of the mean was calculated (SEM).

| | | | | | | |
|---|---|---|---|---|---|---|
| Aspartate | 94.33 ± 26.21 | 104.33 ± 27.34 | 132.67 ± 55.29 | 125.67 ± 68.18 | 279.7 ± 34.17 | 91 ± 18.04 |
| Transaminase U/L | | | | | | |
| Creatine | 119 ± 79.7 | 81.7 ± 25.1 | 54 ± 14.7 | 92.3 ± 29.5 | 266.67 ± 69.9 | 80.7 ± 45.3 |
| PhosphoKinase U/L | | | | | | |
| Creatinine mg/dL | 0.2 ± 0.033 | 0.3 ± 0.067 | 0.3 ± 0 | 0.3 ± 0 | 0.2 ± 0 | 0.2 ± 0 |
| Bilirubin (mg/dL) | 0.1 ± 0.03 | 0.1 ± 0 | 0.1 ± 0 | 0.1 ± 0 | 0.1 ± 0 | 0.2 ± 0.03 |

TABLE 4
Influence of PCLX-001 and doxorubicin treatment on hematology values
in DOHH2 NODscid mouse xenograft model (Supplementary Note 1).

| | | | | | | Doxorubicin |
|---|---|---|---|---|---|---|
| | | | | 50 mg/kg/day | | 3 mg/kg |
| | Vehicle | 10 mg/kg/day | 20 mg/kg/day | Interm. | 50 mg/kg/day | Once a week |
| WBC (K/ul) [1.8-10.7] | 4.38 ± 0.81 | 6.3 ± 0.12 | 5.41 ± 1.05 | 5.94 ± 1.19 | 1.89 ± 0.31 | 8.67 ± 1.58 |
| Absolute Neutrophil | 2. [symbol] ± 0.43 | 4.89 ± 0.34 | 3.94 ± 0.94 | 3.47 ± 0. [symbol] 5 | 1.45 ± 0.22 | 6.86 ± 1.13 |
| cells (K/ul) [0.1-2.4] | | | | | | |
| Absolute Lymphocyte | 0.89 ± [symbol] .27 | 0.83 ± [symbol] .19 | 0.84 ± [symbol] .15 | 1.51 ± 0.47 | 0.23 ± 0.06 | 0.92 ± 0.17 |
| cells (K/ul) [0.9-9.3] | | | | | | |
| Absolute Monocyte | 0.52 ± 0.13 | 0.52 ± 0.06 | 0. [symbol] 1 ± 0.09 | 0.91 ± 0.14 | 0.19 ± 0.06 | 0.85 ± 0.32 |
| cells (K/ul) [0-0.4] | | | | | | |
| Absolute Eosinophil | 0.05 ± 0.02 | 0.05 ± 0.02 | 0.02 ± 0.01 | 0.05 ± 0.03 | 0.01 ± 0.01 | 0.01 ± 0 |
| cells (K/ul) [0-0.2] | | | | | | |
| Absolute Basophil | 0.01 ± 0.00 | 0.01 ± 0 | 0.01 ± 0 | 0 ± 0 | 0 ± 0 | 0.03 ± 0 |
| cells (K/ul) [0-0.2] | | | | | | |
| RBC (M/ul) [6.36-9.42] | 9. [symbol] 2 ± 0.2 | 9.45 ± 0.1 | 9.03 ± 0.32 | 8.07 ± 0.12 | 9.3 ± 0.17 | 8.24 ± 0.35 |
| Hemoglobin (g/dL) | 14.83 ± 0.48 | 14.13 ± 0.18 | 13.97 ± 0.44 | 12.6 ± 0.15 | 14.77 ± 0.23 | 12.73 ± 0.41 |
| [11-15.1] | | | | | | |
| Hematocrit % | 44.8 ± 1.52 | 43.2 ± 0.61 | 42.33 ± 1.48 | 39.53 ± 0.84 | 43.3 ± 0.7 | 38.23 ± 1.97 |
| [35.1-45.4] | | | | | | |
| MCV (fL) [45.4-60.3] | 46.57 ± 0.69 | 45.7 ± 0.2 | 46.9 ± 0.65 | 49.07 ± 1.72 | 46.6 ± 0.2 | 46.36 ± 0.61 |
| MCH (pg) [14.1-19.3] | 15.4 ± 0.2 | 14.93 ± 0.03 | 15.43 ± 0.3 | 15.63 ± 0.12 | 15.9 ± 0.1 | 15.47 ± 0.23 |
| MCHC (g/dL) | 33.13 ± 0.12 | 32.73 ± 0.07 | 33.03 ± 0.22 | 31.90 ± 1.01 | 34.1 ± 0.1 | 33.37 ± 0.92 |
| [30.2-34.2] | | | | | | |
| RDW % [12.4-27] | 25.63 ± 0.68 | 25.97 ± 0.46 | 25.13 ± 0.65 | 27.2 ± 1.82 | 25.8 ± 0.3 | 32.83 ± 0.87 |
| Platelets (K/uL) | 1218.7 ± 211.27 | 1490 ± 91.31 | 1234.7 ± 38.49 | 1673.7 ± 309.81 | 900.7 ± 60.8 | 2210.7 ± 297.9 |
| [592-2972] | | | | | | |
| MPV (fL) [5-20] | 7.23 ± 0.2 | 6.77 ± 0.18 | 7.63 ± 0.23 | 7.17 ± 0.09 | 7 ± 0.3 | 6.2 ± 0.15 |
| Reticulocyte % | 6.82 ± 1.86 | 8.08 ± 1.58 | 7.21 ± 0.49 | 13.28 ± 6.74 | 6.7 ± 0.3 | 15.04 ± 4.87 |

[symbol]

indicates data missing or illegible when filed

Toxicology summary of the DOHH-2 NODscid xenograft (Charles River).

Design: One group of mice were given vehicle and four groups were given PCLX-001 using the dose levels and dose regimens shown in the table below.

| Group | | Dose level (mg/kg) | |
|---|---|---|---|
| no. | Group name | Free base equivalent | Regimen |
| 1 | Vehicle control | [symbol] | Daily for 16 days |
| 2 | Low-dose PCLX- [symbol] 01 | 1 [symbol] | Daily for 16 days |
| 4 | Mid-dose PCLX- [symbol] 1 | 2 [symbol] | Daily for 1 [symbol] days |
| 5 | High-dose PCLX-0 [symbol] 1 | 50 | Every other day for 1 [symbol] days ( [symbol] doses) |
| 6 | High-dose PCLX- [symbol] 01 | 5 [symbol] | Daily for [symbol] days, then [symbol] -day holiday, |
| | | | then daily for 14 days |

[symbol]

indicates data missing or illegible when filed

Mice were observed daily for clinical signs of toxicity and effects on body weight. After the last dose, three mice/group were euthanized and necropsied. At euthanasia, blood samples were taken for hematology analyses and to measure AST and CK activities and bilirubin and creatinine concentrations. At necropsy, samples of samples of femur, both kidneys, liver, small intestine, and injection site were taken and fixed. These were processed and examined microscopically by pathologist Dr. Wei-feng Dong.

Results: The only adverse findings potentially related to PCLX-001 were in the groups given PCLX-001 at 50 mg/kg (Groups 5 and 6). With PCLX-001 every other day, RBC counts were lower than normal in all three mice, and reticulocyte and platelet counts were higher than normal in one of them. With PCLX-001 daily, neutrophil and monocyte counts were lower than normal in all three mice, and monocyte and platelet counts were lower than normal in one of them. There were no histopathologic findings in the femoral bone marrow of any of these mice.

These data are summarized in the table below.

| | | Dose level | | |
|---|---|---|---|---|
| | | (mg/kg) | | |
| Group | Group | Free base | Effect on mean body | |
| no. | name | equivalent | weight | Noteworthy findings after last dose |
| 1 | Vehicle | [symbol] | No change [symbol] | None |
| | control | | | |
| 2 | Low-dose | 10 | No change for [symbol] | None |
| | PCLX-001 | | | |
| 4 | Mid-dose | 20 | No change for [symbol] | None |
| | PCLX-0 [symbol] | | | |
| 5 | High-dose | [symbol] | No change for [symbol] | [symbol] |
| | PCLX [symbol] | | | |
| 6 | High-dose | [symbol] | [symbol] g | [symbol] |
| | PCLX- [symbol] | | | |

[symbol]

indicates data missing or illegible when filed

At the end of the dosing period, serum AST and CK activities were higher-than-normal in one or more mice in each group, including the vehicle control group.

Supplementary Discussion/Conclusions: It is not unusual for mice to sustain some muscle damage (bruising) or liver damage from the handling required to restrain them—for example, to measure tumor size—and this can lead to increased serum AST and/or CK activity. The hematology findings in mice given PCLX-001 at 50 mg/kg were relatively mild and may reflect hematopoietic toxicity, which has been seen in rats and dogs given PCLX-001 at high dose levels⁵⁵.

TABLE 5
Influence of PCLX- [symbol] 1 and doxorubicin treatment on average
organ weight in BL2 NODscid mouse xenograft model (Supplementary
Note 2). Measurements were averaged by treatment group (n = 2).

| | | | | | Doxorubicin |
|---|---|---|---|---|---|
| | | | | | 3 mg/kg |
| | Vehicle | 20 mg/kg/day | 50 mg/kg/day | [symbol] mg/kg/day | Once a week |
| Liver [symbol] | 0.22 | [symbol] | [symbol] | [symbol] | [symbol] |
| Kidney [symbol] | [symbol] | [symbol] | 1.03 | [symbol] | [symbol] |
| Small Intestine (g) | [symbol] | [symbol] | [symbol] | [symbol] | [symbol] |

[symbol]

indicates data missing or illegible when filed

TABLE 6
Influence of PCLX- [symbol] 1 and doxorubicin treatment on serum
chemistry values in BL2 NODscid mouse xenograft model (Supplementary
Note 2). Measurements were averaged by treatment group (n =
3). Standard error of the mean was calculated (SEM).

| | | | | | Doxorubicin |
|---|---|---|---|---|---|
| | | | | | 3 mg/kg |
| | Vehicle | 20 mg/kg/day | 50 mg/kg/day | 80 mg/kg/day | Once a week |
| Alanine [symbol] | [symbol] | [symbol] | [symbol] | [symbol] | [symbol] |
| Aspartate [symbol] | [symbol] | [symbol] | [symbol] | [symbol] | [symbol] |
| Creating Kinase | [symbol] | [symbol] | [symbol] | [symbol] | [symbol] |
| [symbol] | | | | | |
| Blood [symbol] Nitrogen | [symbol] | [symbol] | [symbol] | [symbol] | [symbol] |
| mg/dL | | | | | |
| Creatinine mg/dL | [symbol] | [symbol] | [symbol] | [symbol] | [symbol] |

[symbol]

indicates data missing or illegible when filed

TABLE 7
Influence of PCLX- [symbol] 1 and doxorubicin treatment on hematology values in BL2 NODscid mouse xenograft
model (Supplementary Note 2). Average measurements by treatment group (n = 3). Standard error
of the mean was calculated (SEM). [Normal Range]; Blue for low, Black for normal range, Red for high.

| | | | | | [symbol] |
|---|---|---|---|---|---|
| | Vehicle | [symbol] mg/kg/day | [symbol] mg/kg/day | [symbol] mg/kg/day | Once a week |
| WBC [symbol] | 4.37 ± 1.07 | 4.51 ± 1.44 | 2. [symbol] ± 0.33 | 3.43 ± 0. [symbol] | 4.07 ± 0. [symbol] |
| Absolute Neutrophil cells | 1.63 ± 0.41 | 3.15 ± 1.11 | 1.95 ± 0.25 | 1.44 ± 0.3 | 0. [symbol] ± 0.13 |
| [symbol] | | | | | |
| Absolute Lymphocyte cells | 1.86 ± 0. [symbol] | [symbol] | 0.56 ± 0.14 | 0.42 ± [symbol] | 2.28 ± 0.4 |
| [symbol] | | | | | |
| Absolute Monocyte cells | 0.81 ± 0.1 | [symbol] | 0.26 ± 0.06 | 0.46 ± 0.2 | 0.7 ± 0.19 |
| [symbol] | | | | | |
| Absolute [symbol] cells | 0.0 [symbol] ± 0.0 [symbol] | 0.2 ± 0.0 [symbol] | 0.11 ± 0.05 | 0.0 [symbol] ± 0.05 | 0.0 [symbol] ± 0.03 |
| [symbol] | | | | | |
| [symbol] | [symbol] | [symbol] | [symbol] | [symbol] | [symbol] |
| Neutrophil % | 38.47 ± [symbol] | [symbol] | [symbol] ± 1.46 | [symbol] ± 12.37 | 25.27 ± 3. [symbol] |
| [symbol] | | | | | |
| Lymphocyte % | [symbol] | [symbol] | 19.15 ± 2.71 | 37.17 ± 10.76 | [symbol] |
| [symbol] | | | | | |
| Monocyte % | [symbol] | 10.41 ± 2.55 | [symbol] | [symbol] | [symbol] |
| [symbol] | | | | | |
| [symbol] % | 1.90 ± [symbol] | [symbol] | [symbol] | 3.52 ± 2.49 | 2.18 ± 0. [symbol] |
| [symbol] | | | | | |
| [symbol] % | 0.28 ± 0.17 | 0.27 ± 0.11 | 0.33 ± [symbol] | 0.48 ± 0.39 | 0.37 ± 0.15 |
| [symbol] | | | | | |
| [symbol] | 10.76 ± [symbol] | 8.18 ± 0.28 | 8.94 ± 0.81 | 9.86 ± 0.17 | 10.01 ± 0.13 |
| Hemoglobin (g/dL) | 14.23 ± 0.81 | 11.77 ± [symbol] | 12.4 ± 1 | 13.17 ± 0.65 | 13.6 ± 0.21 |
| [symbol] | | | | | |
| Hematocrit % | [symbol] | [symbol] ± 1.02 | 39.07 ± 3.3 | 46 ± 0.49 | [symbol] ± 0.17 |
| [symbol] | | | | | |
| MCV [symbol] | [symbol] | [symbol] | [symbol] | [symbol] | [symbol] |
| [symbol] | | | | | |
| MCV ( [symbol] L) | 46.17 ± 0.24 | 45.1 ± 0.35 | [symbol] ± 0.47 | 46.67 ± 0.84 | 45.6 ± 0.5 |
| [symbol] | [symbol] | [symbol] | [symbol] | [symbol] | [symbol] |
| MCH [symbol] | 13.23 ± [symbol] | 14.4 ± 0.49 | 13.9 ± 0.15 | 13.33 ± 0.43 | 13.6 ± 0.12 |
| [14.1- [symbol] ] | | | | | |
| MCHC (g/dL) | 28.67 ± 0.37 | 31.83 ± 1.1 | 31.77 ± 0.19 | 28.63 ± [symbol] | [symbol] ± 0.46 |
| [symbol] | | | | | |
| ROW % | 18.3 ± 0.25 | 20.63 ± 0.26 | 20.93 ± 0.32 | 22.73 ± 0.03 | 17.03 ± 0.15 |
| [12.4-27] | | | | | |
| [symbol] | 534 ± 148.85 | 1188.3 ± 158.16 | #z,899 ± 20 [symbol] | 1136.33 ± 246 | 975.6 ± 44.91 |
| [592-2972] | | | | | |
| MPV [symbol] | 5.47 ± 0.13 | 5.8 ± [symbol] | 5.9 ± 0.45 | 5.77 ± 0.18 | 5.7 ± 0.1 |
| [5-20] | | | | | |

[symbol]

indicates data missing or illegible when filed

### Toxicology Summary of the BL2 NODscid Xenograft (Jackson Lab, JAX)

Design: One group of mice were given vehicle and three groups were given PCLX-001 using the dose levels and dose regimens shown in the table below.

| Group | | Dove level | |
|---|---|---|---|
| no. | Group name | (mg/kg) | Regimen |
| 1 | Vehicle control | 0 | Daily for 21 days |
| 2 | Low dose PCLX-001 | 20 | Daily for 26 days |
| 6 | Mid-dose PCLX-001 | 50 | Daily for 21 days |
| 7 | High-doxe PCLX-001 | 60 | Daily for 21 days |

Data collected were the same as in the Charles River xenograft study—clinical signs, body weight, tumor volume, blood samples from 3 mice/group for hematology and clinical chemistry (ALT, AST, BUN, creatinine, CK), and tissue samples collected and fixed from the same 3 mice. Liver, kidneys, and small intestine also were weighed.

Results: Adverse findings potentially related to PCLX-001 were:

Signs of ill health (e.g., rough and scruffy coats, piloerection) in most mice in groups given PCLX-001. These signs developed earlier at 50 or 60 mg/kg/day than at 20 mg/kg/day.

Dehydration and weight loss in groups given PCLX-001 at 50 or 60 mg/kg/day. Weight loss seems to have stopped after about a week, despite continued dosing, after which mice started to gain weight.

Discussion/Conclusions: There were no clinical pathology or anatomic pathology findings related to PCLX-001. There was a trend toward higher neutrophil counts and lower RBC counts with PCLX-001 at 20 mg/kg/day; however, this was unrelated to dose level and so was likely due to chance. Greater mean CK (and to a lesser extent, AST and ALT) activity were seen in one mouse each in Group 1 (control) and Group 4. This pattern of increase in enzyme activities strongly suggests skeletal muscle injury, which was unrelated to PCLX-111.

TABLE 8
Influence of PCLX- [symbol] 1 and doxorubicing treatment on hematology
values in DLBCL NODscid mouse patient derived xenograft model (Supplementary
Note 3) Measurements were averaged by treatment group (n =
3). Standard error of the mean was calculated (SEM). [Normal Range];
Blue for low, Black for normal range, Red for high.

| | Vehicle | 20 mg/kg/day | 50 mg/kg/day |
|---|---|---|---|
| WBC (K/ul) | 2.11 ± 0.19 | 1.49 ± 0.24 | 2.17 ± 0.5 |
| [1.8-10.7] | | | |
| Absolute Neutrophil cells [symbol] | 1.39 ± 0.13 | 0.86 ± 0.22 | 1.28 ± 0.34 |
| [0.1- 2.4] | | | |
| Absolute Lymphocyte cells [symbol] | 0.41 ± [symbol] | [symbol] | 0.52 ± [symbol] .12 |
| [0.9-9.3] | | | |
| Absolute Monocyte cells [symbol] | 0.28 ± 0.03 | 0.23 ± 0.06 | 0.37 ± 0.07 |
| [0-0.4] | | | |
| Absolute [symbol] cells [symbol] | 0.01 ± 0.01 | 0 ± 0 | 0 ± 0 |
| [0-0.2] | | | |
| Absolute Basophil cells [symbol] | 0 ± 0 | 0 ± 0 | 0 ± 0 |
| [0-0.2] | | | |
| Neutrophil % | [symbol] ± 0.72 | 57.17 ± 9.40 | 58.04 ± [symbol] |
| [symbol] | | | |
| Lymphocyte % | [symbol] ± 1.96 | [symbol] | 24.36 ± 2.93 |
| [55.8-91.6] | | | |
| Monocyte % | 14.01 ± [symbol] | 15.48 ± 4.35 | 17.24 ± [symbol] |
| [0-7.5] | | | |
| Eosinophil % | 0.58 ± 0.24 | 0.26 ± 0.08 | 0.32 ± 0.1 |
| [0-3.9] | | | |
| Basophil % | 0.18 ± 0.05 | 0.1 ± 0.04 | 0.04 ± 0.04 |
| [0-2] | | | |
| RBC (M/ul) | 8.86 ± 0.18 | 8.81 ± 0.15 | 8.36 ± 0.83 |
| [ [symbol] .36-9.42] | | | |
| [symbol] (g/dl) | [symbol] ± 0.15 | 11.07 ± 0.27 | 10.4 ± 0.9 |
| [11-15.1] | | | |
| Hematocrit % | [symbol] ± 1.16 | 47.13 ± 1.1 | 42.47 ± 4.04 |
| [ [symbol] -45.4] | | | |
| MCV [symbol] | 52.83 ± 0.34 | 53.5 ± 0.36 | 50.83 ± 0.35 |
| [symbol] | | | |
| MCH (pg) | 12.3 ± 0.1 | 12.57 ± 0.1 [symbol] | 12.5 ± [symbol] |
| [14.1-19.3] | | | |
| MCHC (g/dL) | 23.3 ± 0.29 | 23.5 [symbol] ± 0.38 | 24.53 ± 0.24 |
| [30.2-34.2] | | | |
| RDW % | 17.7 ± 0.46 | 18.07 ± 0.03 | 20.37 ± 0.37 |
| [12.4-27] | | | |
| [symbol] (K/uL) | [symbol] .67 ± 71.52 | 1202.33 ± 76.05 | 859 ± 322.66 |
| [ [symbol] -2972] | | | |
| MPV ( [symbol] ) | 4.97 ± [symbol] | 4.93 ± [symbol] | 5.27 ± 0.18 |
| [5-20] | | | |

[symbol]

indicates data missing or illegible when filed

### Toxicology Summary of the DLBCL3 Patient Derived NODscid Mouse Xenograft

Design: One group of mice were given vehicle and two groups were given PCLX-001 using the dose levels and dose regimens shown in the table below.

| Group | | Dose level | |
|---|---|---|---|
| no. | Group name | (mg/kg) | Regimen |
| 1 | Saline control | 0 | Daily for 21 days |
| 3 | Low-dose PCLX-001 | 20 | Daily for 21 days |
| 5 | High-dose PCLX-001 | 50 | Daily for 21 days |

Data collected were the same as in the previous two studies—clinical signs, body weight, tumor volume, blood samples from 3 mice/group for hematology and clinical chemistry (AST, CK, bilirubin, creatinine), and tissue samples collected and fixed from the same 3 mice.

Results: There were no clinical signs of toxicity, effects on clinical pathology parameters, or anatomic pathology findings related to PCLX-001.

Discussion/Conclusions: The absence of adverse effects is somewhat surprising, since it looked like there were effects on hematology parameters at 50 mg/kg/day in the study using DoHH-2 cells. Why there a difference here is not known. Why mice tolerated daily doses at 50 mg/kg for 3 weeks in this study but not in all studies is not known. Differences including NODscid clones, chow type or microbiota might account for this.

TABLE 9
List of cell lines used in this study

| Cell line | Histology | Site Primary | Hist. [symbol] |
|---|---|---|---|
| [symbol] | [symbol] | [symbol] | [symbol] |
| [symbol] | [symbol] | [symbol] | |
| [symbol] | [symbol] | [symbol] | [symbol] |
| [symbol] | [symbol] | [symbol] | |
| [symbol] | [symbol] | [symbol] | [symbol] |
| [symbol] | [symbol] | [symbol] | [symbol] |
| [symbol] | [symbol] | [symbol] | |
| [symbol] | [symbol] | [symbol] | [symbol] |
| [symbol] | [symbol] | [symbol] | [symbol] |

[symbol]

indicates data missing or illegible when filed

Dose ranging toxicology studies in rat and dog have been performed and reported⁵⁵, and formal GLP toxicology studies in these species are nearing completion in preparation for regulatory review for human clinical trials.

Altogether, our results demonstrate that PCLX-001 treatment inhibits the growth of lymphomas in vivo, including the complete regression of disease refractory to other clinically approved treatments and thus establishes the use of a bonafide NMT inhibitor such as PCLX-001 in cancer.

### Discussion

Herein, we report the discovery that hematological cancer cells, particularly B-cell lymphomas, are highly sensitive to myristoylation inhibition by the novel pan-NMT inhibitor PCLX-001. While the concept of killing cancer cells with a NMT inhibitor has been proposed and tested on small scales^{39, 43, 56, 57, 58, 59}, to our knowledge this work represents the original investigation of the breadth of efficacy of this approach across hundreds of cancer cell lines. We demonstrate that cancer cells can be selectively killed by a NMT inhibitor at concentrations lower than that required to kill and inhibit the proliferation of immortalized and normal cells (FIG. 1E-H, FIGS. 10 and 11). In the absence of additional cytotoxicity assays in more normal cell types, based on the benefit/risk for this therapeutic indication, it is acceptable and not unusual that some normal tissues (e.g. blood cells including PBMCs) are effected at efficacious doses. This indicates a large enough therapeutic window critical to support the development of PCLX-001 as a potential cancer treatment. In addition to inhibiting the myristoylation of a large number of myristoylated proteins in B lymphoma cells (FIG. 2A,B), we demonstrate that PCLX-001 is especially efficient at inhibiting BCR signaling, which is the main lymphoma pro-survival pathway in these cells^{4, 5, 6, 7, 8} In addition, the PCLX-001 BCR signaling inhibition is superior to that of clinically approved SFK inhibitor dasatinib and the BTK inhibitor ibrutinib. This may explain in part why PCLX-001 also has the broadest spectrum of potency against malignant lymphoma cell lines in vitro. We also show PCLX-001 inhibits the myristoylation of SFKs, HGAL and Arf1 and increases their degradation rates, but also unexpectedly promotes the degradation of non-myristoylated pro-survival BCR mediators including P-ERK, NFκB, c-Myc, CREB and perhaps even BTK (FIG. 4A). PCLX-001 treated cells still remained at least 75% viable at concentrations that are becoming cytotoxic. Whether the lower downstream signaling protein levels correspond to a reduction in gene transcription or increased protein degradation in dying cells is not known. Furthermore, PCLX-001 also reduces BCR-mediated calcium mobilization causing apoptosis selectively in B cell lymphoma cells (FIG. 5). The mechanism linking the loss of myristoylation to alterations in calcium homeostasis and inhibition of BCR mediated calcium release is not known.

Increased ER stress is a pro-apoptotic phenomenon previously shown in cells treated with another NMT inhibitor⁵⁹. We postulate the inhibition of myristoylation of the Arf1 GTPase, whether at its N-terminal glycine residue or nearby lysine residue^{36, 37}, interferes with its membrane targeting and impairs vesicle trafficking thereby detrimentally affecting chronic/tonic or antigen dependent BCR signaling. Loss of proper Arf1 functionality at the ER may also explain in part the increase in ER stress marker Bip⁶⁴ upon PCLX-001 treatment (FIG. 4C).

The loss of lipid raft localized myristoylated Lyn (and other SFKs) and HGAL proteins in PCLX-001 treated cells further highlights the importance of these membrane domains in proper BCR signaling^{9, 10, 12, 13, 14}, is (FIG. 5). Furthermore, PCLX-001-mediated myristoylation inhibition of SFKs not only abrogates their membrane targeting but also promotes their degradation via the ubiquitin-proteasome system as MG132 treatments resulted in near complete recovery of SFK levels (FIG. 3B). While ubiquitination and degradation of protein tyrosine kinases by the Casitas B lineage lymphoma (Cbl)-family of E3 ubiquitin ligases^{61, 62} is a normal part of the signal attenuation in B cells, an N-terminal glycine residue has also recently been shown to be a destabilizing factor for proteins, representing a highly selective novel class of N-degron⁶³. Indeed, in their report, Timms et al (2019) demonstrate that unmyristoylated proteins including Lyn, Fyn and Yes, exposing their N-terminal glycine residue are selectively degraded by the N-terminal glycine specific Cullin RING Ligase 2 (CRL2)-ZYGlIB/ZER1 N-degrons-ubiquitin-proteasome system⁶³. This system is highly selective for proteins with a N-terminal glycine residue since substitutions of glycine for any other amino acid led to a substantial stabilization of the resulting proteins⁶³. This newly described N-degron system^{63, 64} may therefore contribute to the faster degradation of unmyristoylated proteins seen in malignant lymphoma cell lines treated with PCLX-001 such as SFKs, HGAL and Arf1 (FIG. 4). It might also explain in part why non-myristoylatable Gly2Ala-Src tyrosine kinase mutant and Gly2Ala-HGAL were previously shown to be more stable than their myristoylated counterpart proteins⁶ 66 since the artificial N-terminal alanine (Ala) residue would prevent the promotion of degradation by the glycine (Gly) residue specific CRL2-ZYG11B or CRL2-ZER1 N-degrons. Thus, we propose a model for the mode of action of PCLX-001 in B-cell lymphoma whereby inhibition of myristoylation of SFKs (or other proteins including HGAL and Arf1) results not only in a loss of membrane targeting but also in a loss of their protein levels and thus function, via the ubiquitin-proteasome system (FIG. 3B), thereby dampening the propagation of BCR signals (FIG. 5). Interestingly, NMT1 was found to be phosphorylated by Lyn, Fyn and Lck SFKs and that phosphorylation of NMT1 was necessary for myristoylation activity since a non-phosphorylatable Y100F-NMT1 mutant lost 98% of its catalytic activity⁶⁷. Therefore, the PCLX-001 mediated loss of SFKs could further reduce NMT1 activity in B lymphoma cells thereby potentiating the loss of pro-survival signals and apoptosis.

In addition to the effects depending on myristoylated SFKs, HGAL and Arf1 proteins, given that there are hundreds of known myristoylated proteins, PCLX-001-mediated effects on lymphoma cell viability likely also occur via the loss of functionality of other myristoylated proteins. Although we still do not know why hematological cancer cells are more vulnerable to PCLX-001 than other cancer cell types, we think this is possibly related to altered expression of either NMT1 or NMT2. Analysis of CCLE NMT1 or NMT2 expression data (FIG. 22) reveals that in addition to be overexpressed in some cancers (aka the current dogma), NAMT expression levels are actually lower in other cancers, many of which are of hematological origin. Altogether, these observations suggest a possible link between the reduction in the number of NMT enzyme targets in hematological cancer cells and the sensitivity of these cells to PCLX-001. Whether altered NMT levels impact on the sensitivity of hematological cancer cells on their own or possibly work in combination with variations in the individual myristoylated proteomes of hematological cancer cells, and, the cell-specific reliance of these cells on various myristoylated proteins for survival is not known. While these possibilities are currently under further investigation in our laboratory, the potential importance of NMT activity to lymphoma cell survival was confirmed in a genome-wide Cas9-Crispr screen in which NMT1 ranked amongst the most critical survival factors in lymphoma cell lines⁶⁸. In addition, our cancer cell line screen results suggest potential for a broader application of PCLX-001 to treatment of leukemia and myeloma, as well as certain solid tumours such as breast and lung cancers.

While PCLX-001 is only marginally efficacious at the tolerated dose of 20 mpk [˜66% tumour reduction (FIG. 7)], we show that it effectively inhibits tumor cell growth in vivo resulting in either major or complete regression of disease in three human lymphoma xenograft models at the 50 mpk efficacious dose, including complete response in a lymphoma refractory to CHOP, Rituximab and other salvage therapies.

### Conclusion

We established that a small molecule NMT inhibitor, PCLX-001, potently and selectively inhibits the growth of a wide spectrum of cultured cancer cells in vitro, with particularly pronounced effects in cells derived from hematologic cancers including B-cell lymphoma due to the loss of BCR-mediated signaling events, their main source of pro-survival signals^{4, 5, 6, 7, 8}. Together with the striking efficacy of PCLX-001 in pre-clinical models of B-cell lymphoma in vivo, these findings support the ongoing development and potential clinical trials of PCLX-001 and related NMT inhibitors as therapies for B cell lymphoma and possibly other cancers.

### Methods

Rabbit anti-PARP-1 (1:500, affinity purified polyclonal #EU2005, lot 1), anti-GAPDH (1:5000, affinity purified polyclonal, #EU1000, lot 1) and anti-GFP (1:10000, affinity purified, #EUl, lot B3-1) were from laboratory stock and are available through Eusera (www.eusera.com). Our affinity purified rabbit anti-GFP is also available as Ab6556 from Abcam (Cambridge, MA). Rabbit monoclonal anti-Src (1:2000, clone 32G6, #2123, lot 5), Lyn (1:2000, clone C13F9, #2796, lot 4), P-Lyn Y567 (1:5000, polyclonal, #2731, lot 5), Fyn (1:2000, polyclonal, #4023, lot 3), Lck (1:2000, clone D88, #2984, lot 4), Hck (1:2000, clone ElJ7F, #14643, lot 1), c-Myc (1:10000, clone D3N8F, #13987, lot 5), ERK (1:2000, clone 4695, #9102, lot 27), P-ERK (1:5000, clone 3516, #9161, lot 36), P-SFK (1:10000, clone D49G4, #6943, lot 4), BTK (1:2000, clone D3H5, #8547, lot 13), P-BTK Y223 (1:5000, clone D9T6H, #87141, lot 1) SYK (1:2000, clone D3Z1E, #13198, lot 5), P-SYK Y525/526 (1:5000, clone C87C1, lot 18) and anti-cleaved caspase-3 (1:1000, clone 5AlE, #9664, lot 20) were purchased from Cell Signaling Technologies. Rabbit monoclonal anti-BIP (1:2000, polyclonal, ADI-SPA-826) was purchased from Enzo Life Sciences. Rabbit anti-Mcl-1 (1:2000, clone Y37, #32687, lot GR119342-5), NFκB (1:2000, clone E379, #32536, lot GR3199609-2), P-Lyn Y396 (1:5000, polyclonal, #226778, lot GR3195652-5) were purchased from Abcam (Cambridge, MA). Mouse monoclonal anti-p-Tyr (1:10000, PY99, sc-7020, lot 12118) antibody was purchased from Santa Cruz Biotechnology. Mouse anti human HGAL was purchased at eBioscience (1:10000, clone 1H1-A7, #14-9758-82, lot E24839-101). Rabbit polyclonal anti-ARF-1 antibody (1:2000, polyclonal, #PAl-127, lot TK 279638) was purchased from ThermoFisher Scientific. Enhanced chemiluminescence (ECL) Prime Western blotting detection kits were purchased from GE Healthcare. Clarity ECL western blotting substrate was from Bio-Rad. Goat anti-Human IgM (μ chain) (70-8028-M002, lot S728028002001) was purchased from Tonbo biosciences. Goat F(ab′)2 anti-human IgM was purchased from BioRad (STAR146, lot 152684). Rabbit Anti-human Src antibody from Sigma-Aldrich (polyclonal, Ab-529, lot 871521168) was used for immunoprecipitation. Doxorubicin hydrochloride was from Pfizer. Dasatinib and ibrutinib were from ApexBio Technology. PCLX-001 was identified as DDD86481 by Drs. David Gray and Paul Wyatt (University of Dundee, Scotland, UK)^{38, 69}. All chemicals were of the highest purity available and purchased from Sigma-Aldrich, unless indicated otherwise.

### Cell Culture

IM9, Ramos, SU-DHL-10 and COS-7 were purchased from ATCC. BL2, DOHH2, WSU-DLCL2 & BJAB were purchased from DSMZ (Germany). Ramos and BL2 were kind gifts of Drs. Jim Stone and Robert Ingham of University of Alberta. VDS isolation was described in Tosato G, et al. (reference 47). VDS, BJAB and SU-DHL-10 were kind gifts of Dr. Michael Gold of the University of British Columbia. HUVEC cells (pooled from up to 4 umbilical cords) were purchased from PromoCell. All cell lines identity was confirmed by STR profiling at The Genetic Analysis Facility, The Centre for Applied Genomics, The Hospital for Sick Children, Peter Gilgan Centre for Research and Learning, 686 Bay St., Toronto, ON, Canada M5G 0A4 (www.tcag.ca). Cell lines were tested regularly for mycoplasma contamination using MycoAlert Plus Mycoplasma Detection Kit (Lonza, ME, USA). All cell lines tested negative for mycoplasma contamination. All cell lines were maintained in RPMI or DMEM medium supplemented with 5-10% fetal bovine serum, 100 U/ml penicillin, 0.1 mg/ml streptomycin, 1 mM sodium pyruvate, and 2 mM L-glutamine. HUVEC cells (pooled from up to 4 umbilical cords) were purchased from PromoCell and cultured in Endothelial cell growth media with Insulin-like Growth Factor (Long R3 IGF) and Vascular Endothelial Growth Factor (VEGF) and maintained at passages lower than 7. All cell lines were maintained at 37° C. and 5% CO₂ in a humidified incubator and routinely checked for the presence of contaminating mycoplasma. Please see supplementary Table 3 for cell line names, types and histology. For transfections, adherent cells COS-7 cells were transfected using X-tremeGENE9 DNA (Roche) transfection reagent according to manufacturer's instructions. For BCR activation experiments, cells were incubated with 25 μg/ml of Goat F(ab′)₂ anti-human IgM (or anti-human IgM (μ chain) showing identical BCR activation properties) for 2 minutes and the activation was stopped by the addition of 1 mM vanadate (Bio Basic Inc) solution in PBS.

### Lysis of Cells

Cells were harvested, washed in cold PBS, and lysed in 6.1% SDS-RIPA buffer (50 mM Tris-HCl pH 8.0, 150 mM NaCl, 1% Igepal CA-630, 0.5% sodium deoxycholate, 2 mM MgCL₂, 2 mM EDTA with 1×complete protease inhibitor; (Roche Diagnostics) by rocking for 15 min at 4° C. The lysates were centrifuged at 16,000 g for 10 min at 4° C., and the post-nuclear supernatant was collected.

Immunoblotting, Immunoprecipitation and Metabolic Labelling of Cells with Alkyne-Myristate

Protein concentrations were determined by BCA assay (Thermo Scientific) according to manufacturer's instructions. Samples were prepared for electrophoresis by the addition of 5× loading buffer and boiled for 5 min. If not stated otherwise, 30 g of total protein per lane is loaded on a 12.5% acrylamide gels. After electrophoresis, gels are transferred onto 0.2 μM nitrocellulose membrane (Bio-Rad) thereafter probed with antibodies as described in materials section. Peroxidase activity is revealed following the procedure provided for the ECL Prime Western Blotting Detection Reagent (GE Healthcare, PA, USA).

Immunoprecipitation was performed as previously described in Yap et al.⁴⁷. Briefly, cells are washed with cold PBS, harvested, and lysed with cold EDTA-free RIPA buffer (0.1% SDS, 50 mM HEPES, pH 7.4, 150 mM NaCl, 1% Igepal CA-630, 0.5% sodium deoxycholate, 2 mM MgCl₂, EDTA-free complete protease inhibitor (Roche)) by rocking for 15 min at 4° C. Cell lysates are centrifuged at 16,000 g for 10 min at 4° C. and the post-nuclear supernatants are collected. EGFP fusion proteins or endogenous c-Src non-receptor tyrosine kinase (Src) were immunoprecipitated from approximately 1 mg of protein lysates with affinity purified goat anti-GFP (www.eusera.com) or rabbit anti-Src antibody (Sigma, Ab-529, lot 871521168) by rocking overnight at 4° C. Pure proteome protein G magnetic beads (Millipore) were incubated with immunoprecipitated proteins for 2 h and extensively washed with 6.1% SDS-RIPA, re-suspended in 1% SDS in 50 mM HEPES, pH 7.4 and heated for 15 min at 80° C. The supernatants containing the immunoprecipitated proteins were collected for Western blot analysis or click chemistry.

IM9, BL2 and COS-7 cells were treated with PCLX-001 for 1 h and cells were then labelled with 25 μM ω-alkynyl myristic acid 30 min before harvesting at each time point. Protein from the resulting cell lysates were reacted with 100 μM azido-biotin using click chemistry and processed as described in Yap et al.⁴⁷ and Perinpanayagam et al.³³

Viability of Cells Treated with PCLX-001, Dasatinib and Ibrutinib

IM9, VDS, BL2, Ramos, BJAB, DOHH2, WSU-DLCL2, and SU-DHL-10 cells (1×10⁵ cells) were grown in six-well plates in 4 ml media/well and incubated with increasing concentrations of PCLX-001, dasatinib and ibrutinib for up to 96 hrs. Viability of cells treated with PCLX-001 was measured by CellTiter-Blue Cell Viability Assay (Promega) or with calcein AM staining (Life Technologies) according to the manufacturer's instructions on a Cytation 5 plate reader (Biotek, Winooski, VT). Calcein assay consists of measuring the cell viability ratio (live cells/total cells and expressed as % viability). Cells were stained with the Nuclear-ID Blue/Red cell viability reagent (GFP-certified, Enzo Life Sciences) to identify total cells, and dead cells while live cells were stained with Calcein AM (Life Technologies) according to manufacturer's instructions. Cell count was performed using a Cytation 5 Cell Imaging Multi-Mode Reader (Biotek Instruments, Inc.) and analysed by Biotek Gen5 Data Analysis software (version 2.09).

Cell viability was also measured using the Horizon (St. Louis, MO) platform. Cells were seeded in growth media in black 384-well tissue culture treated plates at 500 cells per well. Cells are equilibrated in assay plates via centrifugation and placed in incubators at 37° C. for 24 h before treatment. At the time of treatment, a set of assay plates (which do not receive treatment) are collected and ATP levels are measured by adding ATPLite● (Perkin Elmer, Waltham, MA). These Tzero (To) plates are read using ultra-sensitive luminescence on Envision plate readers. Assay plates are incubated with compound for 96 h (except where noted in Analyzer) and are then analyzed using ATPLite●. All data points are collected via automated processes and are subject to quality control and analyzed using Horizon's Chalice Analyzer proprietary software (1.5). Assay plates were accepted if they passed the following quality control standards: relative raw values were consistent throughout the entire experiment, Z-factor scores were greater than 06.6 and untreated/vehicle controls behaved consistently on the plate. Horizon utilizes Growth Inhibition (GI) as a measure of cell growth. The GI percentages are calculated by applying the following test and equation:

$$If T < {V}_{0} : 100 * ( 1 - \frac{T - {V}_{0}}{{V}_{0}} ) \\ If T \geq {V}_{0} : 100 * ( 1 - \frac{T - {V}_{0}}{V - {V}_{0}} )$$

where T is the signal measure for a test article, V is the untreated/vehicle-treated control measure, and Vₒ is the untreated/vehicle control measure at time zero (also colloquially referred as T₀ plates). This formula is derived from the Growth Inhibition calculation used in the National Cancer Institute's NCI-60 high throughput screen. 100% GI therefore represents complete growth inhibition (cytostasis) while 200% GI represents complete cell death.

Cell viability was also measured using the Oncolines (Netherlands Translational Research Center B.V.) platform. Cells were diluted in the corresponding ATCC recommended medium and dispensed in a 384-well plate, depending on the cell line used, at a density of 200-6400 cells per well in 45 μl medium. For each used cell line the optimal cell density is used. The margins of the plate were filled with phosphate-buffered saline. Plated cells were incubated in a humidified atmosphere of 5% CO₂ at 37° C. After 24 hours, 5 μL of compound dilution was added and plates were further incubated. At t=end, 24 μL of ATPlite 1Step™ (PerkinElmer) solution was added to each well, and subsequently shaken for 2 minutes. After 10 minutes of incubation in the dark, the luminescence was recorded on an Envision multimode reader (PerkinElmer).

Finally, 3^{rd} breadth of PCLX-001 efficiency screen (FIG. 9) was performed using the ChemPartner platform (Shanghai, China). 131 cell lines were seeded in 96-well plate, black wall, tissue culture treated (from Corning, Cat.3904) and cultured following ATCC formulation. Cell viability after 72 hrs and 144 hrs was measured using Cell Titer Blue Viability Assay (from Promega, Cat. G8081, Lot. No. 0000190181) and fluorescence at 560/590 nm was recorded with Enspire (PerkinElmer). EC₅₀ was calculated using XLfit software (5.5).

### Cell Proliferation Assay

Proliferation of cells was measured by imaging and counting after digital phase contrast picture transformation for better accuracy. 2×10⁵ cells were cultured in six-well plates in 4 ml of culture media and incubated with increasing concentration of PCLX-001. After homogenization, 50 μl of culture was transferred into a high binding clear glass bottom ½ area 96 well plate (Greiner bio-one). Total well area was imaged in bright field (12 stitched pictures) using a Cytation 5 Cell Imaging Multi-Mode Reader (Biotek Instruments, Inc.) and transformed into a single digital phase contrast picture. Total cell counts were performed daily for up to 4 days (Biotek Gen5 Data Analysis software 2.09).

### Intracellular Calcium Measurements:

Cytosolic free calcium concentration measurements were performed in BL2 lymphoma cells incubated for 24 h or 48 h with 1 μM PCLX-001, dasatinib or ibrutinib using PTI fluorometer (Photon Technology International) using adapted previously described protocol⁵³ 10.10⁶ cells are suspended in fresh media with 8 μM Fura-2 AM (Molecular Probes) and 1 mM CaCl₂ for 30 minutes, washed and resuspended in media supplemented with calcium for an additional 15 minutes. Cells are then washed and resuspended in warm Krebs Ringer solution (10 mM HEPES pH 7.0, 140 mM NaCl, 4 mM KCl, ImM MgCl₂ and 10 mM glucose) and placed in a four-sided clear cuvette. Prior to activation, the free cytoplasmic calcium was chelated with 0.5 mM EGTA for 1 minute. BCR receptor dependent calcium release is activated by the addition of 10 μg/ml Goat F(ab′)₂ anti Human IgM (BioRad). Following, Thapsigargin (300 nM) was used to show BCR-independant and irreversible Ca2+ release from the endoplasmic reticulum. Ca2+ concentrations were calculated with the following equation:

[Ca⁺⁺]=Kd(R−Rmin)/(Rmax−R)

with R=Fluorescence Intensity at 340 nm divided by fluorescence intensity at 380 nm, Rmax=fluorescence measured following Ionomycin (7.5 μM) and CaCl2 (12 mM) addition, Rmin=fluorescence measured following EGTA (32 mM), Tris (24 mM) and Triton™ X-100 (0.4%) and Kd=224 (at 37^{C} for Fura-2 AM).

Results shown are representative of multiple replicates of the experiment (n=6 for PCLX-001 incubation, n=3 for Dasatinib and Ibrutinib).

### Isolation of PBMC and Lymphocytes and Cell Viability Assay

2 healthy human research volunteers were recruited for PBMC and lymphocytes isolation from a 20 ml blood collection (patient #1: male, 34 years old, no diagnosis, no treatment; patient #2: male, 54 years old, no diagnosis, no treatment). Study protocol was approved by the Health Research Ethics Board of Alberta Cancer Committee (Study title: Evaluations of Fatty AcylTransferases (FATs) in fresh blood and blood forming cells; HREBA.CC-17-0624).

Mononuclear cells were isolated from peripheral blood by density gradient centrifugation using Ficoll-Paque (GE Healthcare, PA, USA). Lymphocytes were isolated from whole blood samples using EasySep™ lymphocyte isolation kit (Stemcell Technologies, Vancouver, BC, Canada) as per manufacturer's instructions. PBMC and lymphocytes were cultured in RPMI medium with 10% FBS, 100 U/ml penicillin, 0.1 mg/ml streptomycin. Cells were plated at a concentration of 2×10⁶ cells/ml. After incubation with 0.001-0.1 μM PCLX-001 for 96 hrs, cell viability was measured by using CellTiter-Fluor™ viability assay (Promega, Madison, WI, USA).

### Immunohistochemistry

COS-7 cells were cultured plated on Poly-d-Lysine-coated 35-mm glass-bottom dishes (MatTek Corporation, Ashland, MA, USA) and transiently transfected with the indicated fluorescently tagged proteins using X-tremeGENE9 DNA (Roche) as recommended by the suppliers. Images were acquired using a Zeiss Observer Z1 microscope and Axiovision software (Axiovision, version 4.8). B-cell lymphomas were fixed in formalin, embedded in paraffin, cut into 5 mm sections with a microtome, mounted on Superfrost Plus slides (Fisher Scientific), deparaffinized with xylene (3 times for 10 min each), dehydrated in a graded series of ethanol (100%, 80% and 50%), and washed in running cold water for 10 min.

For antigen retrieval, slides were loaded in a slide holder and placed in a Nordicware microwave pressure cooker. 800 ml 10 mM citrate buffer pH 6.0 was added, and the pressure cooker was tightly closed and microwaved on high for 20 min. The slides were washed in cold running water for 10 min, soaked in 3% H₂O₂ in methanol for 10 min, and washed with warm running water for 10 min and with PBS for 3 min. Excess PBS was removed and a hydrophobic circle was drawn around the sample with a PAP pen (Sigma-Aldrich, St. Louis, MO). Anti-cleaved caspase 3 or anti-Ki-67 were diluted with Dako antibody diluent buffer (1:50, ˜400 μml per slide), and incubated in a humidity chamber overnight at 4° C. Slides were washed in PBS twice for 5 min each and ˜4 drops of EnVision+System-HRP labelled polymer (anti-rabbit) (Dako, Agilent Technologies, Santa Clara, CA) was added to each slide and incubated at room temperature for 30 min. Slides were washed again in PBS twice for 5 min each, and 4 drops of liquid diaminobenzidine+substrate chromogen (prepared according to manufacturer's instructions; Dako, Agilent Technologies) was added. The slides were developed for 5 min and rinsed under running cold water for 16 min. The slides were then soaked in 1% CuSO₄ for 5 min, rinsed briefly with running cold water, counterstained with haematoxylin for 60 sec, and rinsed with running cold water. Next, slides were dipped in lithium carbonate 3 times, rinsed, and dehydrated in a graded series of ethanol. Coverslips were added, and the slides were examined with a Nikon Eclipse 80i microscope and photographed with a QImaging camera.

### Ethics Approval

We have complied with all relevant ethical regulations for human, animal testing and research. All relevant experiments in this study have received the appropriate ethical approval. The name of board and/or institution that approved the study protocol are described below.

Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International, which assures compliance with accepted standards for the care and use of laboratory animals.

In Vivo Services at The Jackson Laboratory—Sacramento facility, an OLAW-assured and AAALAC-accredited organization conducted the DOHH2 mouse xenograft study. It was performed according to an IACUC-approved protocol and in compliance with the Guide for the Care and Use of Laboratory Animals (National Research Council, 2011).

For the study using DLBCL lymphocytes, all procedures were approved and carried out in accordance with the guiding ethical principles of the Institutional Review Board of the Singapore General Hospital (SGH). Written informed consent was obtained for use of these samples for the specific research purpose only. The experimental protocol (#130812) was approved by the Institutional Animal Care and Use Committee (IACUC) of the Biological Resource Center (BRC), A*STAR. All procedures involving human samples were approved by and performed in accordance with the ethics principles of the Sing Health Centralized Institutional Review Board. Written informed consent was obtained for use of these samples for the specific research purpose only.

### Xenograft Studies in Mice

DOHH2 xenograft study at Charles River's facility: Female severe combined immunodeficient mice (Fox Chase SCID®, C.B-17/Icr-Prkdcscid/IcrIcoCrl, Charles River) were nine weeks old on Day 1 of the study and had a BW range of 17.8-22.9 g. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. On Day 1 of the study, animals were given a rehydration solution ad libitum in an effort to reduce dehydration during the dosing phase of the study. The rehydration solution consisted of 0.45% NaCl: 2.5% glucose; and 0.075% KCl in sterile water. The mice were housed on irradiated Enrich-o'Cobs™ bedding in static microisolators on a 12-hour light cycle at 20-22° C. (68-72° F.) and 40-60% humidity.

BL2 xenograft study at Jackson Laboratory: One hundred five (105) 6 week old female NOD.CB17-Prkdc scid/J (NOD scid, Stock #001303) mice were transferred to the in vivo research laboratory in Sacramento, CA. The mice were ear notched for identification and housed in individually and positively ventilated polysulfone cages with HEPA filtered air at a density of 5 mice per cage. Initially cages were changed every two weeks. The animal room was lighted entirely with artificial fluorescent lighting, with a controlled 12 hour light/dark cycle (6 am to 6 pm light). The normal temperature and relative humidity ranges in the animal rooms were 20-26° C. and 30-70%, respectively. The animal rooms were set to have up to 15 air exchanges per hour. Filtered tap water, acidified to a pH of 2.5 to 3.0, and standard lab chow were provided ad libitum.

BL2 or DOHH-2 cells (1×10⁷) and a cell suspension containing neoplastic DLBCL lymphocytes isolated from the pleural fluid of consented patient DLBCL3 were subcutaneously injected into the flank of immuno-compromised, female, NODscid mice at the Jackson Laboratory's, Charles River's, and Singapore General Hospital's facilities, respectively. After tumors formed, mice were divided into groups of approximately 10 animals and given subcutaneous injections of vehicle daily, PCLX-001 daily at 10-60 mg/kg, or doxorubicin weekly at 3 mg/kg⁷⁰, as indicated in each figure. The dose volume was 10 mL/kg. At the end of the two-to three-week dosing period, mice were euthanized and three/group were necropsied. Mice that died or were euthanized early for humane reasons also were necropsied. In life, mice were monitored regularly and weighed daily, and tumors were measured with digital Vernier calipers (Mitutoyo) every other day. Tumor volume was calculated as length (mm)×width (mm)2/2; length and width were the longest and shortest diameters, respectively. At euthanasia, at the end of the dosing period blood samples were taken for hematology analyses and clinical chemistry analyses that included AST and CK activities and bilirubin and creatinine concentrations (plus ALT activity and BUN concentration in the Jackson Laboratory study). At necropsy, samples of femur, both kidneys, liver, small intestine, and injection site were collected and fixed. These were subsequently processed and examined by light microscopy for histopathologic findings. Also at necropsy, the tumors were removed and divided in two. One piece was fixed in 10% neutral buffered formalin for 24 h at room temperature and embedded in paraffin; the other was snap frozen for RNA and protein analysis. Tumor growth inhibition (TGI) for all xenograft experiments was calculated following the formula:

TGI (%)=(V_{control}−V_{treated})/(V_{control}−_{Vnitial})*100.

### Patient Derived Xenograft Mouse Studies:

### i) Patient Data

Patient DLBCL3 was a 58 year-old male who had been treated for Stage I diffuse large B-cell lymphoma at age 43 with cyclophosphamide, doxorubicin, vincristine, and prednisolone (CHOP), which resulted in complete remission (Supplementary Table 2). Patient DLBCL3 then presented to Singapore General Hospital 10 years subsequently with recurrent disease in the bone marrow and leptomeninges and pleural effusions. He received two courses of rituximab, ifosfamide, carboplatin, and etoposide and intrathecal methotrexate/cytarabine, followed by four courses of dexamethasone, cytarabine, and cisplatin and intrathecal methotrexate. His tissue was harvested for PDX propagation at this time. His disease continued to progress, and he died a year later.

### ii) Pathology

Cytological examination of the pleural fluid showed discohesive lymphomatous population featuring large cells with vesicular chromatin and conspicuous nucleoli. Neoplastic cells expressed pan-B markers (PAX5, CD20, CD22, CD79a), with aberrant expression of CD5, strong expression of bcl2, and a high proliferation fraction (70-80%). Neoplastic lymphocytes had a nongerminal centre phenotype (negative for CD10 and positive for bcl6, MUM1, FOXP1) but staining for c-Myc was low (20%). Interphase fluorescence in situ hybridization showed gains of BCL2 and rearrangements of BCL6 and IGH; normal patterns were seen for C-MYC. RNA in situ hybridization showed lack of NMT2 expression.

iii) Xenograft Construction and Treatment

The pleural fluid was collected in cold sterile 20% RPMI 1640 medium and neoplastic cells were isolated with Ficoll-Paque Plus (GE Healthcare) and re-suspended in RPMI 160 medium (Life Technologies) with 20% foetal bovine serum (Life Technologies, Carlsbad, CA). A representative part of the tumor sample was fixed in 10% neutral buffered formalin; the other part was used for xenotransplantation. The cell suspension was injected subcutaneously in the flank of 4-6-week-old NODscid mice. When the tumors reached a maximum of 1000 mm³, the mice were sacrificed, tumors were harvested, and a necropsy was performed. Xenograft tumors were immediately frozen, fixed in formalin, and stored in 90% foetal bovine serum, and 10% dimethyl sulfoxide or placed in RPMI 1640 medium. This process was repeated to produce subsequent generations of patient-derived xenograft models (P2, P3, P4, . . . ). To evaluate the maintenance of the morphology and main characteristics of the tumor of origin, formalin-fixed, paraffin-embedded tissue sections from patient tumor samples and xenografts of all established patient-derived xenograft models were stained with haematoxylin and eosin. These sections were also immunostained to measure the expression of various markers. A clinical pathologist reviewed all the slides. For the current study, tumor fragments (˜50 mg, P4) were implanted subcutaneously in the flank of 4-6-week-old female NODscid mice and allowed to grow to 200-300 mm³. The mice were then randomized into groups (n=8 per group) and injected subcutaneously with vehicle (10 ml/kg); PCLX-001, 20 mg/kg daily for 21 days; or PCLX-001, 50 mg/kg daily for 18 days, with a 3-day break after 9 days. Tumor measurements and growth inhibition calculations were performed as described above.

For the DLBCL3 PDX study, NODscid mice were purchased from InVivos, Singapore and fed with standard laboratory diet and distilled water ad libitum. The animals were kept on a 12 h light/dark cycle at 22±2° C. in BRC, A*STAR and maintained in accordance with the institutional guidelines.

### NMT Activity Assay

NMT activity assay was described in Perinpanayagam et al.³³. Briefly, cells were lysed and sonicated (10 sec) in sucrose buffer (50 mM NaH₂PO₄, pH 7.4, and 0.25M sucrose). Tumor samples were cut into small pieces, extracted by glass Dounce homogenization (12 full strokes) in sucrose buffer, and sonicated (10 sec). The protein lysates were incubated with 0.1 mM of myristoylatable or non-myristoylatable decapeptide corresponding to the N-terminal sequence of p60-Src and 12 pM of [³H]-myristoyl-CoA (Perkin Elmer, Waltham, MA) in NMT assay buffer (0.26M Tris-HCl pH 7.4, 3.25 mM EGTA, 2.92 mM EDTA and 29.25 mM 2-mercaptoethanol, 1% Triton X-100) in 25 μl reactions and incubated for 15 min at 30° C. The reaction was terminated by spotting 15 μl of the reaction mixture onto a P81 phosphocellulose paper disc (Whatman, Maidstone, UK), washed and processed for scintillation counting.

### Statistical Methods

Data were analyzed using Prism 8 software (GraphPad, version 8.4.1) and generally expressed as mean+s.e.m. Statistical significance was determined using Student t-test or one-way ANOVA when applicable. Analysis of the significance of drug treatments on tumor volume was assessed by 2-way ANOVA. P values higher than 0.05 were not considered statistically significant. (***) P≤0.001, (**) P≤0.01 and (*) P≤0.05.

Statistical analysis of NMT1 and NMT2 expression: NMT1 and NMT2 mRNA expression data were extracted on Mar. 26 2020 from the Broad Institute CCLE database⁵⁴ (https://portals.broadinstitute.org/ccle) and contained the mRNA expression data for 1269 cancer cell lines. The RNAseq TPM gene expression data (Expression Public 20Q1) were analyzed for protein coding genes using RSEM and are presented as Log₂ transformed values using a pseudo-count of 1 (FIG. 22).

T cell Receptor (TCR) Activation

Jurkat T cells were purchased from ATCC. Cells were maintained in RPMI medium supplemented with 5% fetal bovine serum, 100 U/ml penicillin, 0.1 mg/ml streptomycin at 37° C. and 5% CO₂ in a humidified incubator and routinely checked for the presence of contaminating mycoplasma. For TCR activation experiments, PCLX-001 pretreated cells were incubated with 2 μg/ml of CD3 and CD28 monoclonal antibodies (ThermoFisher Scientific, Cat #14-0037-82 and #14-0281-82 respectively) for various times (optimal activation after 15-60 minutes) and the activation was stopped by the addition of 1 mM vanadate (Bio Basic Inc) solution in PBS. Cells were harvested, washed in cold PBS, and lysed in 4.1% SDS-RIPA buffer (50 mM Tris-HCl pH 8.0, 150 mM NaCl, 1% Igepal CA-630, 0.5% sodium deoxycholate, 2 mM MgCl₂, 2 mM EDTA with 1× complete protease inhibitor; (Roche Diagnostics) by rocking for 15 min at 4 C. The lysates were centrifuged at 16,000 g for 10 min at 4° C., and the post-nuclear supernatant was collected and analyzed by immunoblotting.

### REFERENCES

1. WHO-IARC. Global Cancer Observatory.) (2019).
2. Noone A M H N, Krapcho M, Miller D, Brest A, Yu M, Ruhl J, Tatalovich Z, Mariotto A, Lewis D R, Chen H S, Feuer E J, Cronin K A. SEER Cancer Statistics Review.).
3. Beveridge R, et al. Economic impact of disease progression in follicular non-Hodgkin lymphoma. Leuk Lymphoma 52, 2117-2123 (2011).
4. Vaque J P, et al. B-cell lymphoma mutations: improving diagnostics and enabling targeted therapies. Haematologica 99, 222-231 (2014).
5. Burger J A, Wiestner A. Targeting B cell receptor signalling in cancer: preclinical and clinical advances. Nat Rev Cancer 18, 148-167 (2018).
6. Corso J, et al. Elucidation of tonic and activated B-cell receptor signaling in Burkitt's lymphoma provides insights into regulation of cell survival. Proceedings of the National Academy of Sciences of the United States of America 113, 5688-5693 (2016).
7. Schmitz R, et al. Burkitt lymphoma pathogenesis and therapeutic targets from structural and functional genomics. Nature 490, 116-120 (2012).
8. Young R M, Staudt L M. Targeting pathological B cell receptor signalling in lymphoid malignancies. Nat Rev Drug Discov 12, 229-243 (2013).
9. Cheng P C, Dykstra M L, Mitchell R N, Pierce S K. A role for lipid rafts in B cell antigen receptor signaling and antigen targeting. J Exp Med 190, 1549-1560 (1999).
10. Gupta N, DeFranco A L. Lipid rafts and B cell signaling. Semin Cell Dev Biol 18, 616-626 (2007).
11. Tauzin S, et al. Oncogenic association of the Cbp/PAG adaptor protein with the Lyn tyrosine kinase in human B-NHL rafts. Blood 111, 2310-2320 (2008).
12. Kovarova M, Tolar P, Arudchandran R, Draberova L, Rivera J, Draber P. Structure-function analysis of Lyn kinase association with lipid rafts and initiation of early signaling events after Fecepsilon receptor I aggregation. Mol Cell Biol 21, 8318-8328 (2001).
13. Kurosaki T, Hikida M. Tyrosine kinases and their substrates in B lymphocytes. Immunol Rev 228, 132-148 (2009).
14. Slupsky J R. Enhancing BCR signals at the cell membrane. Blood 125 , 586-587 (2615).
15. Natkunam Y, et al. Expression of the human germinal center-associated lymphoma (HGAL) protein, a new marker of germinal center B-cell derivation. Blood 105, 3979-3986 (2005).
16. Mano H. Tec family of protein-tyrosine kinases: an overview of their structure and function. Cytokine Growth Factor Rev 10, 267-280 (1999).
17. Scharenberg A M, Humphries L A, Rawlings D J. Calcium signalling and cell-fate choice in B cells. Nat Rev Immunol 7, 778-789 (2007).
18. Berridge M J, Bootman M D, Roderick H L. Calcium signalling: dynamics, homeostasis and remodelling. Nat Rev Mol Cell Biol 4, 517-529 (2003).
19. Scuoppo C, et al. Repurposing dasatinib for diffuse large B cell lymphoma. Proceedings of the National Academy of Sciences of the United States of America 116, 16981-16986 (2019).
20. Jerkeman M, Hallek M, Dreyling M, Thieblemont C, Kimby E, Staudt L. Targeting of B-cell receptor signalling in B-cell malignancies. J Intern Med 282, 415-428 (2017).
21. Duronio R J, Towler D A, Heuckeroth R O, Gordon J I. Disruption of the yeast N-myristoyl transferase gene causes recessive lethality. Science 243, 796-800 (1989).
22. Giang D K, Cravatt B F. A second mammalian N-myristoyltransferase. The Journal of biological chemistry 273, 6595-6598 (1998).
23. Martin D D, Beauchamp E, Berthiaume L G. Post-translational myristoylation: Fat matters in cellular life and death. Biochimie 93, 18-31 (2011).
24. Wright M H, Heal W P, Mann D J, Tate E W. Protein myristoylation in health and disease. J Chem Biol 3, 19-35 (2010).
25. Castrec B, et al. Structural and genomic decoding of human and plant myristoylomes reveals a definitive recognition pattern. Nat Chem Biol 14, 671-679 (2018).
26. Bologna G, Yvon C, Duvaud S, Veuthey A L. N-Terminal myristoylation predictions by ensembles of neural networks. Proteomics 4, 1626-1632 (2004).
27. Eisenhaber F, et al. Prediction of lipid posttranslational modifications and
localization signals from protein sequences: big-Pi, NMT and PTSl. Nucleic Acids Res 31, 3631-3634 (2003).
28. Maurer-Stroh S, Eisenhaber B, Eisenhaber F. N-terminal N-myristoylation of proteins: prediction of substrate proteins from amino acid sequence. J Mol Biol 317, 541-557 (2002).
29. Zha J, Weiler S, Oh K J, Wei M C, Korsmeyer S J. Posttranslational N-myristoylation of BID as a molecular switch for targeting mitochondria and apoptosis. Science 290, 1761-1765 (2000).
30. Vilas G L, Corvi M M, Plummer G J, Seime A M, Lambkin G R, Berthiaume L G. Posttranslational myristoylation of caspase-activated p21-activated protein kinase 2 (PAK2) potentiates late apoptotic events. Proceedings of the National Academy of Sciences of the United States of America 103, 6542-6547 (2006).
31. Martin D D, et al. Tandem reporter assay for myristoylated proteins post-translationally (TRAMPP) identifies novel substrates for post-translational myristoylation: PKCepsilon, a case study. FASEB J 26, 13-28 (2012).
32. Martin D D, et al. Rapid detection, discovery, and identification of post-translationally myristoylated proteins during apoptosis using a bio-orthogonal azidomyristate analog. FASEB J 22, 797-806 (2008).
33. Perinpanayagam M A, Beauchamp E, Martin D D, Sim J Y, Yap M C, Berthiaume L G. Regulation of co- and post-translational myristoylation of proteins during apoptosis: interplay of N-myristoyltransferases and caspases. FASEB J 27, 811-821 (2013).
34. Ducker C E, Upson J J, French K J, Smith C D. Two N-myristoyltransferase isozymes play unique roles in protein myristoylation, proliferation, and apoptosis. Mol Cancer Res 3, 463-476 (2005).
35. Haun R S, Tsai S C, Adamik R, Moss J, Vaughan M. Effect of myristoylation on GTP-dependent binding of ADP-ribosylation factor to Golgi. The Journal of biological chemistry 268, 7064-7068 (1993).
36. Kosciuk T, et al. NMT1 and NMT2 are lysine myristoyltransferases regulating the ARF6 GTPase cycle. Nat Commun 11, 1067 (2020).
37. Dian C, et al. High-resolution snapshots of human N-myristoyltransferase in action illuminate a mechanism promoting N-terminal Lys and Gly myristoylation. Nat Commun 11, 1132 (2020).
38. Frearson J A, et al. N-myristoyltransferase inhibitors as new leads to treat sleeping sickness. Nature 464, 728-732 (2010).
39. Kallemeijn W W, et al. Validation and Invalidation of Chemical Probes for the Human N-myristoyltransferases. Cell Chem Biol 26, 892-900 e894 (2019).
46. Magnuson B A, Raju R V, Moyana T N, Sharma R K. Increased N-myristoyltransferase activity observed in rat and human colonic tumors. J Natl Cancer Inst 87 , 1630-1635 (1995).
41. Raju R V, Moyana T N, Sharma R K. N-Myristoyltransferase overexpression in human colorectal adenocarcinomas. Experimental cell research 235, 145-154 (1997).
42. Selvakumar P, Smith-Windsor E, Bonham K, Sharma R K. N-myristoyltransferase 2 expression in human colon cancer: cross-talk between the calpain and caspase system. FEBS Lett 580, 2021-2026 (2006).
43. Selvakumar P, Lakshmikuttyamma A, Shrivastav A, Das S B, Dimmock J R, Sharma R K. Potential role of N-myristoyltransferase in cancer. Prog Lipid Res 46, 1-36 (2007).
44. Rajala R V, Radhi J M, Kakkar R, Datla R S, Sharma R K. Increased expression of N-myristoyltransferase in gallbladder carcinomas. Cancer 88, 1992-1999 (2000).
45. Shrivastav A, Varma S, Senger A, Khandelwal R L, Carlsen S, Sharma R K. Overexpression of Akt/PKB modulates N-myristoyltransferase activity in cancer cells. J Pathol 218, 391-398 (2009).
46. Tosato G, et al. Epstein-Barr virus immortalization of normal cells of B cell lineage with nonproductive, rearranged immunoglobulin genes. J Immunol 137, 2637-2642 (1986).
47. Yap M C, et al. Rapid and selective detection of fatty acylated proteins using omega-alkynyl-fatty acids and click chemistry. J Lipid Res 51, 1566-1580 (2010).
48. McCabe J B, Berthiaume L G. Functional roles for fatty acylated amino-terminal domains in subcellular localization. Mol Biol Cell 10, 3771-3786 (1999).
49. Davis R E, et al. Chronic active B-cell-receptor signalling in diffuse large B-cell lymphoma. Nature 463, 88-92 (2010).
50. Ku M, et al. Src family kinases and their role in hematological malignancies. Leuk Lymphoma 56, 577-586 (2015).
51. Nijhawan D, et al. Elimination of Mcl-1 is required for the initiation of apoptosis following ultraviolet irradiation. Genes Dev 17, 1475-1486 (2003).
52. Nguyen P H, Niesen E, Hallek M. New roles for B cell receptor associated kinases: when the B cell is not the target. Leukemia 33, 576-587 (2019).
53. Prins D, Groenendyk J, Touret N, Michalak M. Modulation of STIM1 and capacitative Ca2+ entry by the endoplasmic reticulum luminal oxidoreductase ERp57. EMBO Rep 12, 1182-1188 (2011).
54. Barretina J, et al. The Cancer Cell Line Encyclopedia enables predictive modelling of anticancer drug sensitivity. Nature 483, 603-607 (2012).
55. Weickert M, et al. Initial Characterization and Toxicology of an Nmt Inhibitor in Development for Hematologic Malignancies. Blood 134, 3362-3362 (2019). https://doi.org/16.1182/blood-2019-124934
56. Das U, Kumar S, Dimmock J R, Sharma R K. Inhibition of protein N-myristoylation: a therapeutic protocol in developing anticancer agents. Curr Cancer Drug Targets 12, 667-692 (2012).
57. Thinon E, Morales-Sanfrutos J, Mann D J, Tate E W. N-Myristoyltransferase Inhibition Induces E R-Stress, Cell Cycle Arrest, and Apoptosis in Cancer Cells. ACS Chem Biol 11, 2165-2176 (2016).
58. Kim S, et al. Blocking Myristoylation of Src Inhibits Its Kinase Activity and Suppresses Prostate Cancer Progression. Cancer Res 77, 6950-6962 (2017).
59. Kim S, et al. Myristoylation of Src kinase mediates Src induced and high fat diet accelerated prostate tumor progression in mice. The Journal of biological chemistry, (2017).
60. Citterio C, Vichi A, Pacheco-Rodriguez G, Aponte A M, Moss J, Vaughan M. Unfolded protein response and cell death after depletion of brefeldin A-inhibited guanine nucleotide-exchange protein GBF1. Proceedings of the National Academy of Sciences of the United States of America 105, 2877-2882 (2008).
61. Shao Y, Yang C, Elly C, Liu Y C. Differential regulation of the B cell receptor-mediated signaling by the E3 ubiquitin ligase Cbl. The Journal of biological chemistry 279, 43646-43653 (2004).
62. Mohapatra B, et al. Protein tyrosine kinase regulation by ubiquitination:
critical roles of Cbl-family ubiquitin ligases. Biochimica et biophysica acta 1833, 122-139 (2013).
63. Timms R T, Zhang Z, Rhee D Y, Harper J W, Koren I, Elledge S J. A glycine-specific N-degron pathway mediates the quality control of protein N-myristoylation. Science 365, (2019).
64. Eldeeb M, Esmaili M, Fahlman R. Degradation of proteins with N-terminal glycine. Nat Struct Mol Biol 26, 761-763 (2019).
65. Patwardhan P, Resh M D. Myristoylation and Membrane binding regulate c-Src Stability and Kinase Activity. Mol Cell Biol, (2010).
66. Lu X, et al. HGAL localization to cell membrane regulates B-cell receptor signaling. Blood 125, 649-657 (2015).
67. Rajala R V, et al. Phosphorylation of human N-myristoyltransferase by N-myristoylated SRC family tyrosine kinase members. Biochemical and biophysical research communications 288, 233-239 (2001).
68. Phelan J D, et al. A multiprotein supercomplex controlling oncogenic signalling in lymphoma. Nature 560, 387-391 (2018).
69. Brand S, et al. Lead optimization of a pyrazole sulfonamide series of Trypanosoma brucei N-myristoyltransferase inhibitors: identification and evaluation of CNS penetrant compounds as potential treatments for stage 2 human African trypanosomiasis. J Med Chem 57, 9855-9869 (2014).
70. Jones L W, et al. Effects of exercise training on antitumor efficacy of doxorubicin in MDA-MB-231 breast cancer xenografts. Clin Cancer Res 11, 6695-6698 (2005).

### Example 2

FIG. 23 PCLX-001 treatment attenuates TCR dependent P-ERK activation in Jurkat T cells. Jurkat T cells were activated with CD3/CD28 antibodies for up to 60 minutes (2 ug/ml). Immunoblotting analysis shows that PCLX-001 incubated for 24/48 h (1 μM) inhibit P-ERK activation.

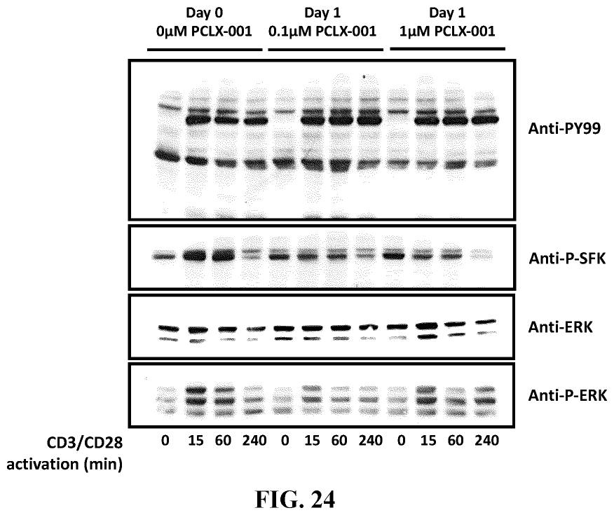

FIG. 24 PCLX-001 treatment (24 h) attenuates TCR dependent P-ERK and P-SFK activation in Jurkat T cells. Jurkat T cells were activated with CD3/CD28 antibodies for up to 4 hours (2 ug/ml). Immunoblotting analysis shows that PCLX-001 incubated for 24 h (0.1 and 1 μM) P-ERK activation and phosphorylation of Src family kinases (P-SFK).

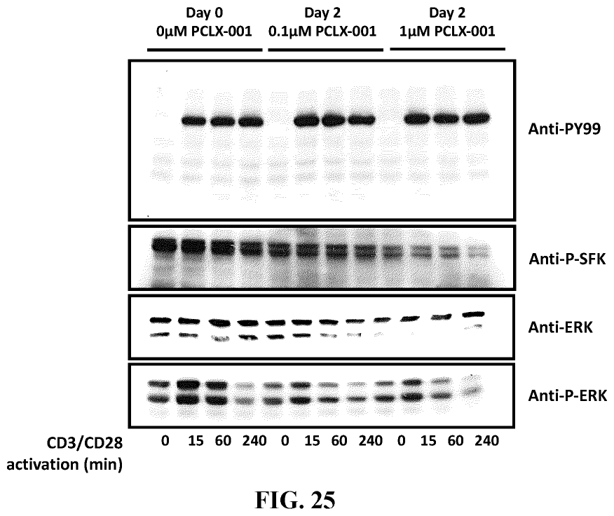

FIG. 25 PCLX-001 treatment (48 h) attenuates TCR dependent P-ERK and P-SFK activation in Jurkat T cells. Jurkat T cells were activated with CD3/CD28 antibodies for up to 4 hours (2 ug/ml). Immunoblotting analysis shows that PCLX-001 incubated for 48 h (0.1 and 1 μM) inhibit P-ERK activation and phosphorylation of Src family kinases (P-SFK).

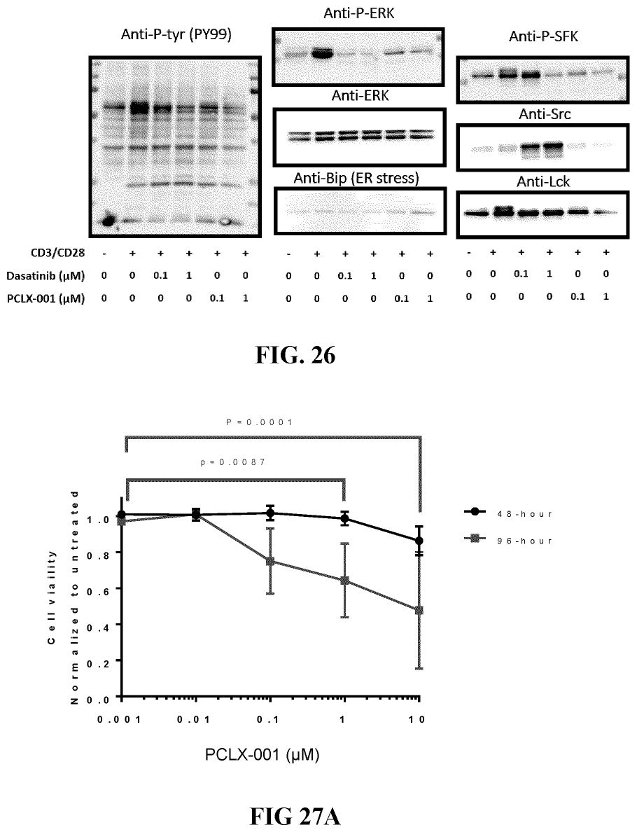

FIG. 26 PCLX-001 and Dasatinib treatment attenuates TCR downstream signaling events and induce ER stress in primary cultured T cells. 90% αb primary T cells were activated with CD3/CD28 antibodies for 30 min (2 ug/ml). Immunoblotting analysis shows that PCLX-001 and Dasatinib inhibit P-tyrosine phosphorylation (PY99), P-ERK activation, phosphorylation of Src family kinases (P-SFK). In addition, PCLX-001 reduced the protein level of Src and Lyn significantly and increased Bip protein content (ER stress marker).

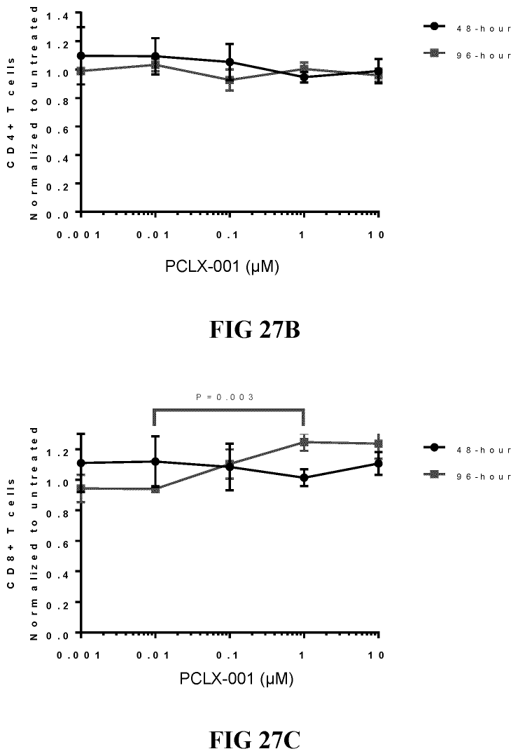

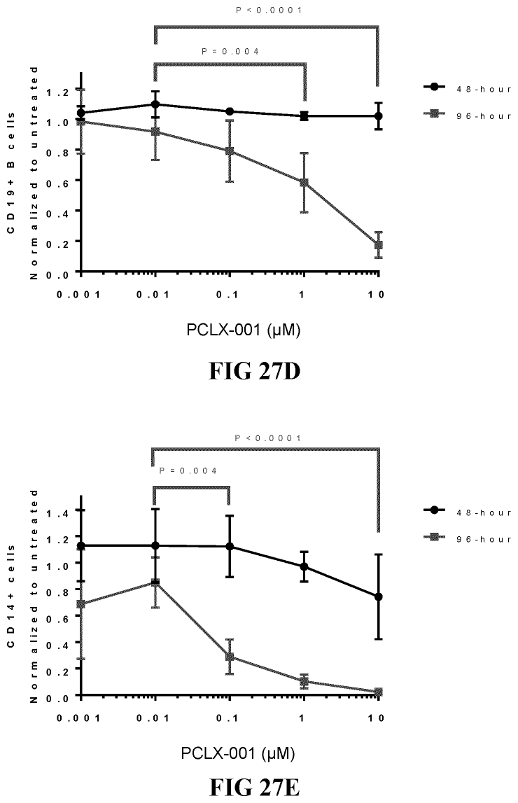

FIG. 27A-E PCLX-001 reduces the viability of PBMC, B cells and monocytes but not T cells. PBMC were cultured for 4 days in the presence of increasing concentrations of PCLX-001 (0-10 ug/ml). the viability and the abundance of cell subset were tested using flow cytometry. The viability of PBMC was markedly reduced (A). Although the frequency of CD4+ and CD8+ T cells was not changed by the drug treatment (B and C). However, B cells (D) and monocyte CD14+ (E) numbers were significantly decreased after 96 hours of PCLX-001 treatment.

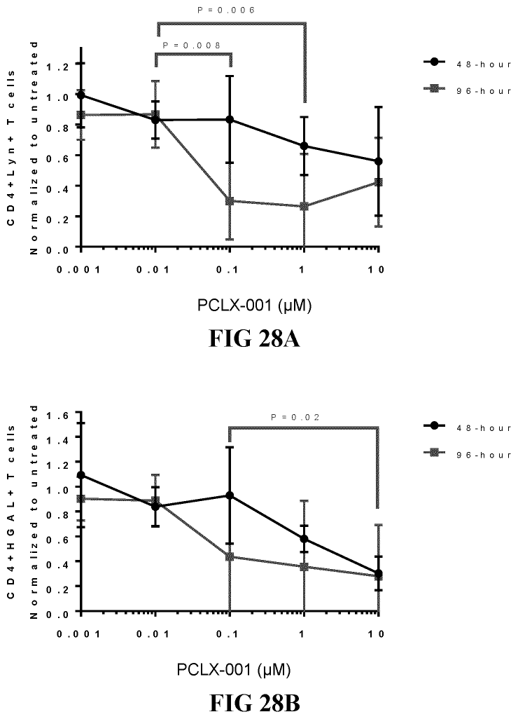

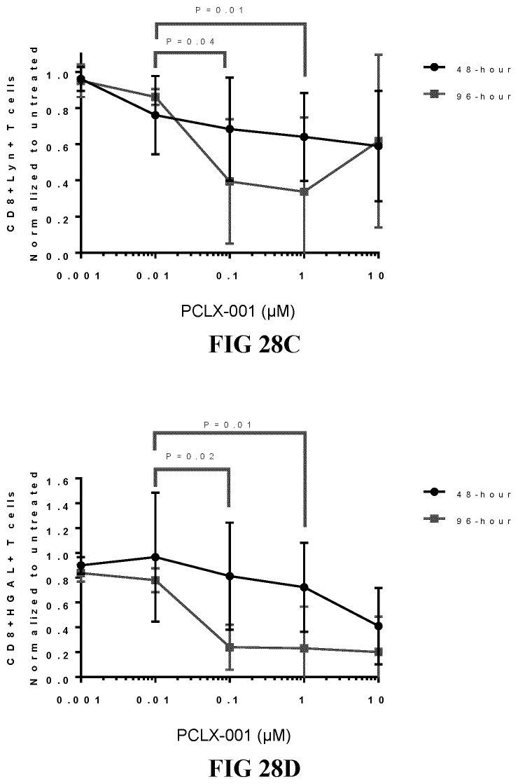

FIG. 28A-D PCLX-001 reduces the expression of Lyn and HGAL in T cells.

PBMC were cultured for 4 days in the presence of increasing concentrations of PCLX-001 (0-10 ug/ml). The expression of Lyn and HGAL in T cell subset were tested using intracellular staining through flow cytometry. The expression of Lyn (A) and HGAL (B) in CD4+ T cells were both decreased. In addition, PCLX-001 also reduced the expression of both Lyn (C) and HGAL (D) in CD8+ T cells.

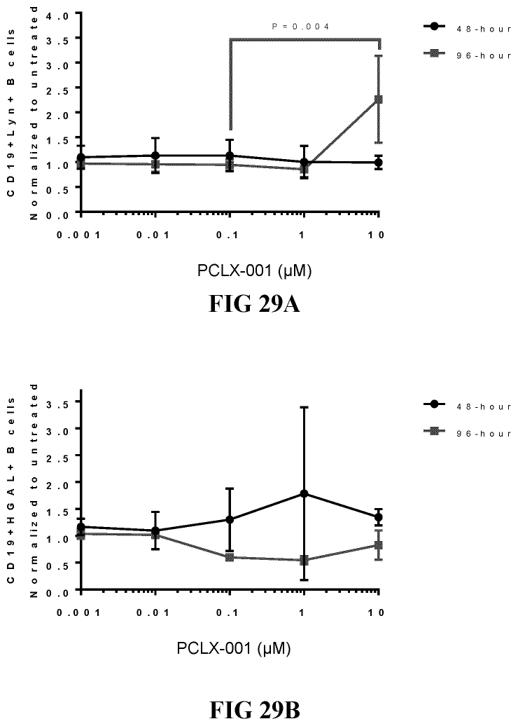

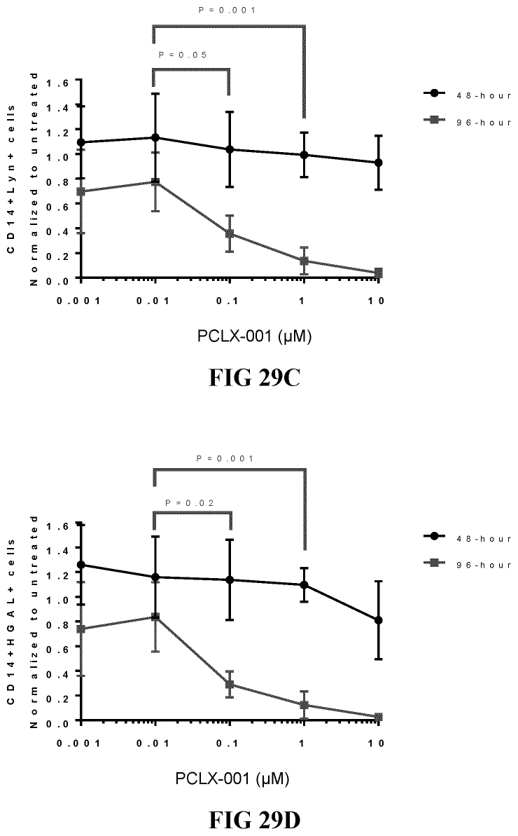

FIG. 29A-D PCLX-001 reduces the expression of Lyn and HGAL in monocytes but not in B cells. PBMC were cultured for 4 days in the presence of increasing concentrations of PCLX-001 (0-10 ug/ml). The expression of Lyn and HGAL in B cells and monocyte subset were tested using intracellular staining through flow cytometry. Although PCLX-001 couldn't reduce the expression of Lyn (A) and HGAL (B) in B cells, both protein markers were significantly reduced in monocytes (C and D).

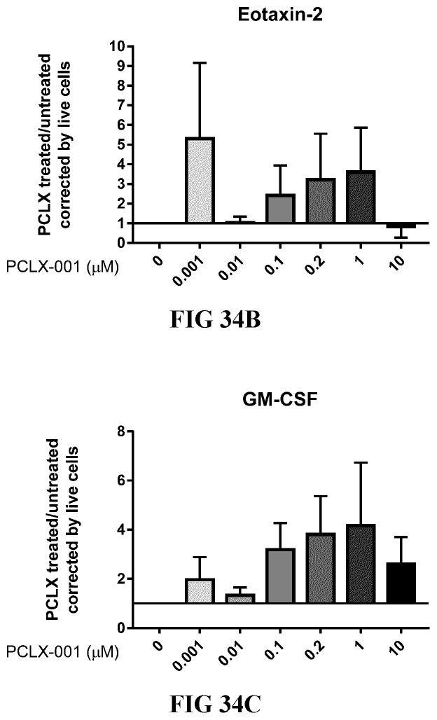

FIG. 34A-E PCLX-001 induces the production of inflammatory cytokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory cytokines IL-6 (A), TNF-α (B), IL-8 (C), IFN-γ (D), and IL-17a (E) in live PBMC.

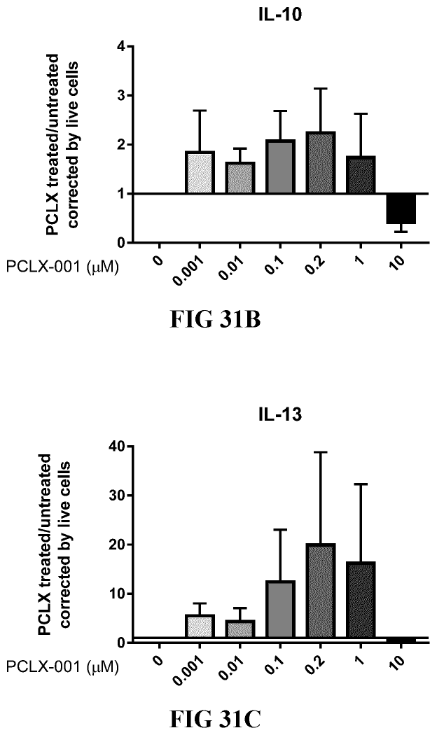

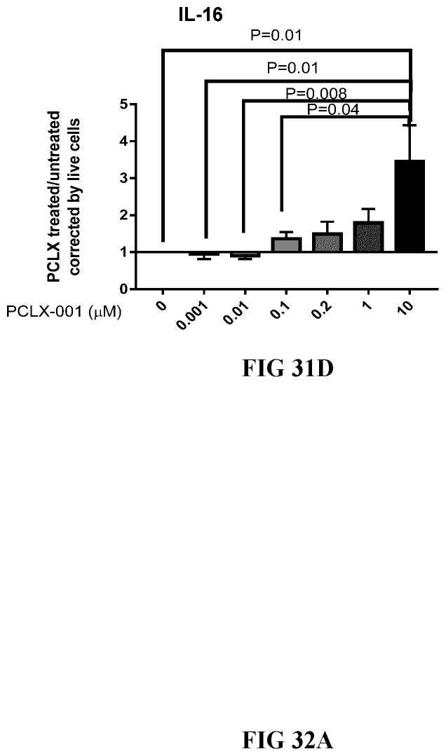

FIG. 31A-D PCLX-001 induce the production anti-inflammatory cytokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the anti-inflammatory cytokines IL-IRA (A), IL-10 (B), IL-13 (C), and IL-16 (D) in live PBMC.

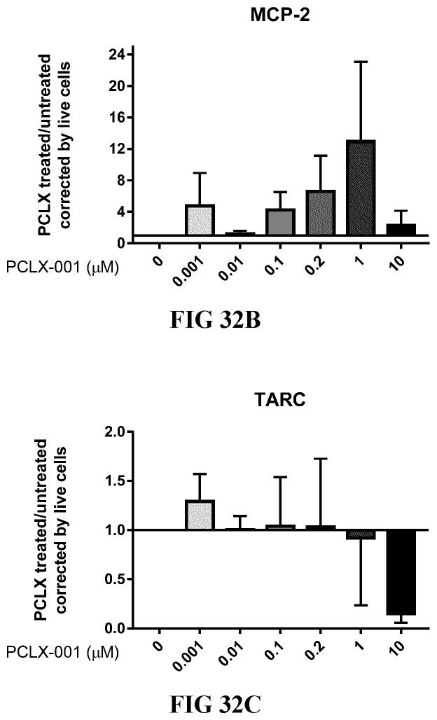

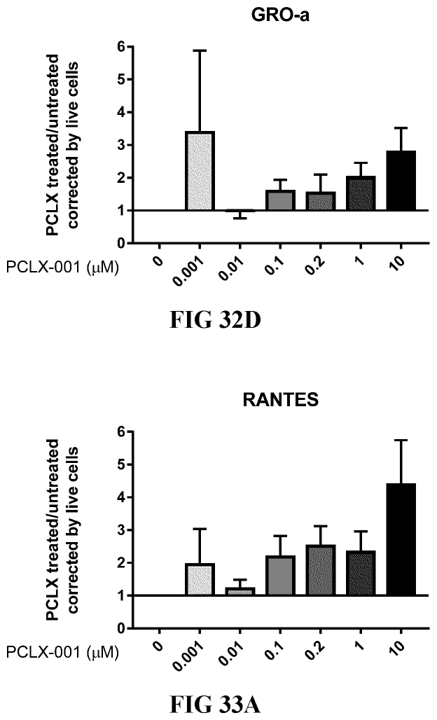

FIG. 32 A-D PCLX-001 induce the production of inflammatory chemokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory chemokines MIP-1α (A), MCP-2 (B), TARC (C), and GRO-α (D) in live PBMC.

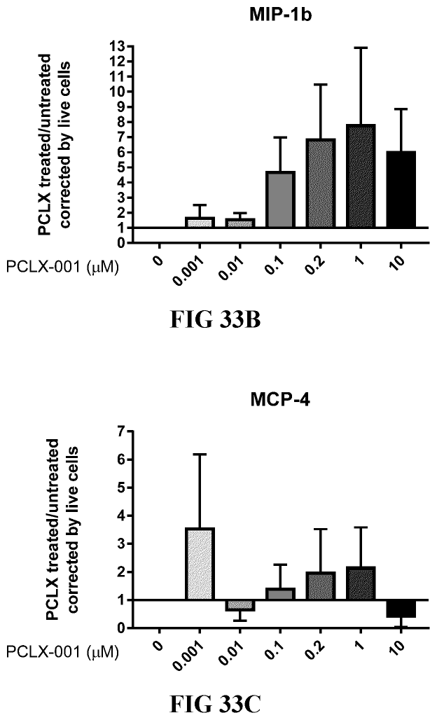

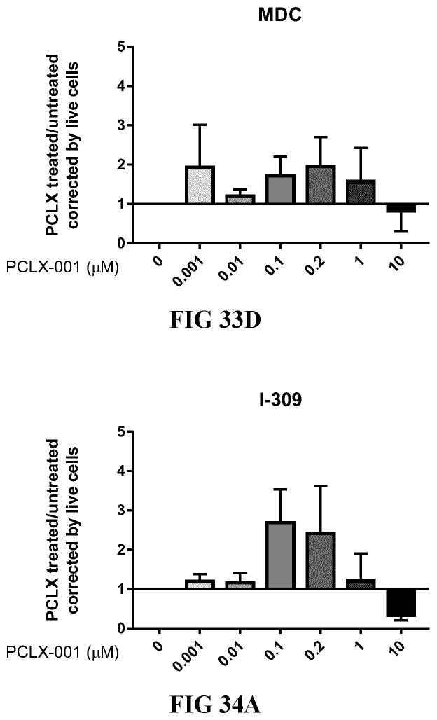

FIG. 33A-D PCLX-001 induce the production of inflammatory chemokines. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the inflammatory chemokines RATES (A), MIP-10 (B), MCP-4 (C), and MDC (D) live PB<C.

FIG. 34A-C PCLX-001 induce the production of T helper 2-mediated chemokines and GM-CSF. PBMC were cultured for 4 days in the presence of increasing concentrations of PCLX-001 (0-10 ug/ml). After 4 days the cell culture supernatant was analysed for various biomarkers using multiplex cytokine array (Eve Technologies Discovery assay, Calgary, CA) human cytokine/chemokine 71-Plex (HD71). PCLX-001 induce the production of the granulocyte-monocyte colony stimulating factor I-309 (A), Eotaxin-2 (B) as T helper 2 mediated chemokines and GM-CSF (C) in live PBMC.

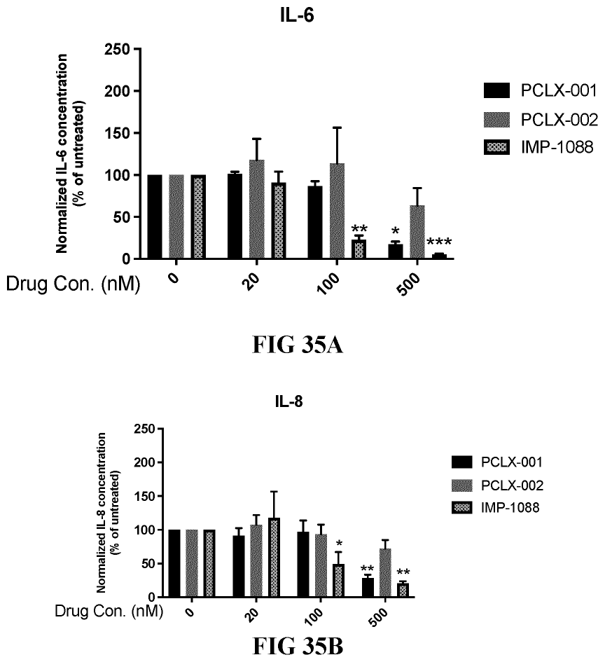

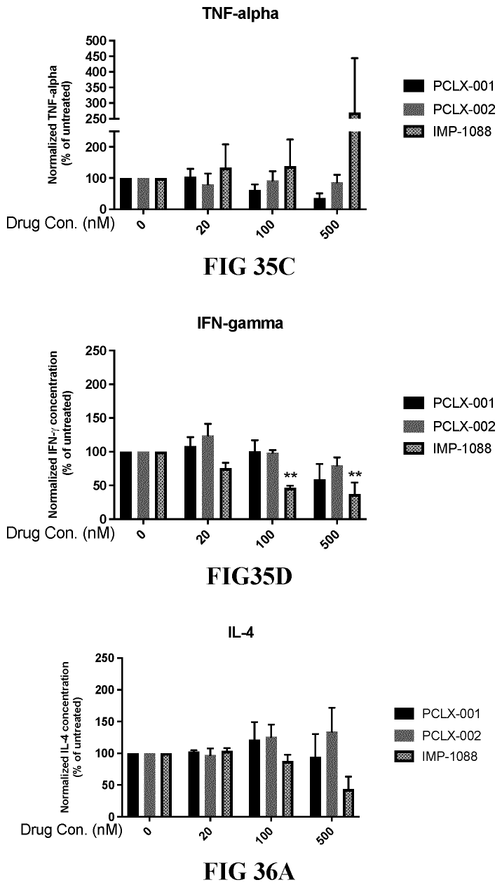

FIG. 35A-D NMT inhibitors (PCLX-001, PCLX-002, IMP-1088) reduce the normalized secretion of pro-inflammatory cytokines; IL-6 (A), IL-8 (B), TNF-α (C), and IFN-γ (D). T cells were incubated for 48 h with increasing concentration of NMT inhibitors, then induced by T cell activator (STEMCELLS) in the presence of the drugs for 2 more days. NMT inhibitors significantly reduced the level of IL-6, IL-8 and IFN-gamma. (Two-way ANOVA, P value against untreated: *<0.05-0.01 **<0.01-0.001 *<0.001-0.0001. It is noteworthy to mention that reduction of cytokine secretion is stronger in the more potent NMT inhibitor PCLX-001 than PCLX-002 and that the survival of cells after 4 days of treatment was within 10% of untreated samples.

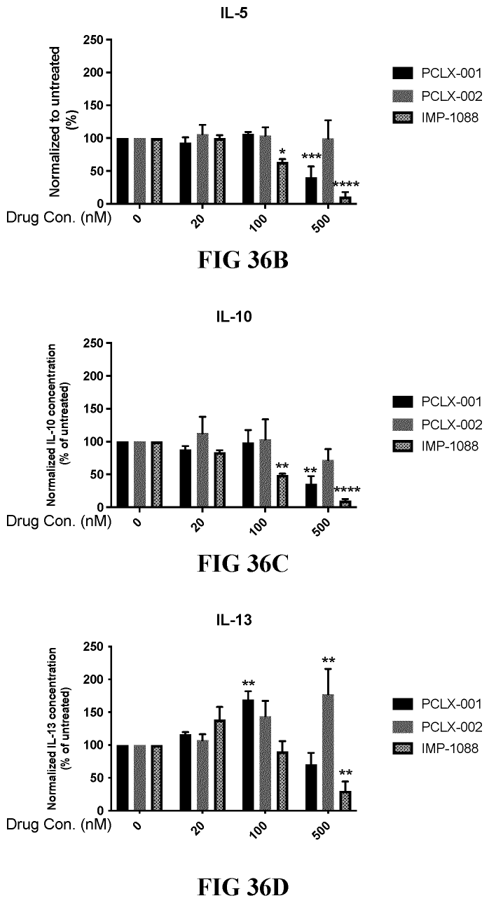

FIG. 36A-D NMT inhibitors (PCLX-001, PCLX-002, IMP-1088) reduce the normalized secretion of anti-inflammatory cytokines; IL-4 (A), IL-5 (B), IL-14 (C), and IL-13 (D). T cells were incubated for 48 h with increasing concentration of NMT inhibitors, then induced by T cell activator (STEMCELLS) in the presence of the drugs for 2 more days. NMT inhibitors significantly reduced the level of IL-5, IL-14 and IL-13.(Two-way ANOVA, P value against untreated: *<0.05-0.01 **<0.01-0.001 *<0.001-0.0001. It is noteworthy to mention that reduction of cytokine secretion is stronger in the more potent NMT inhibitor PCLX-001 than PCLX-002 and that the survival of cells after 4 days of treatment was within 10% of untreated samples.

FIG. 23-25 depicts the effect of PCLX-001 and Dasatinib on TCR pathway in Jurkat T cells activated with CD3/CD28 antibodies for 30 min (2 ug/ml).

FIG. 26 depicts the effect of PCLX-001 and Dasatinib on TCR pathway in 90% ab primary T cells activated with CD3/CD28 antibodies for 30 min (2 ug/ml).

FIG. 27 depict viability of different hematological cells subset in presence of increasing amount of PCLX-001.

FIGS. 28-29 depict the amount of myristoylated proteins Lyn and HGAL in different hematological cells subset in presence of increasing amount of PCLX-001.

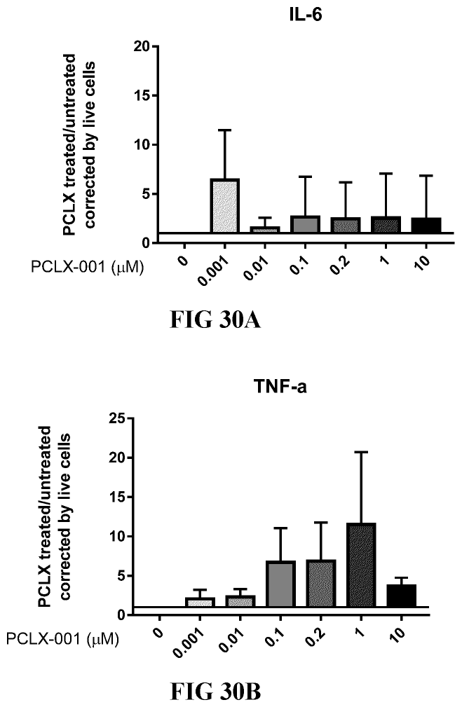

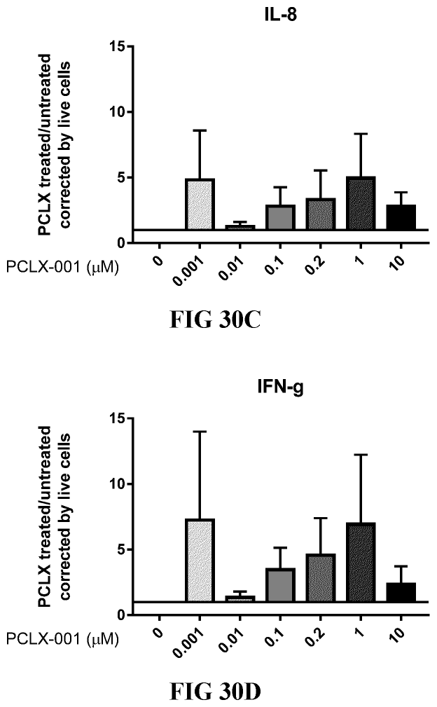

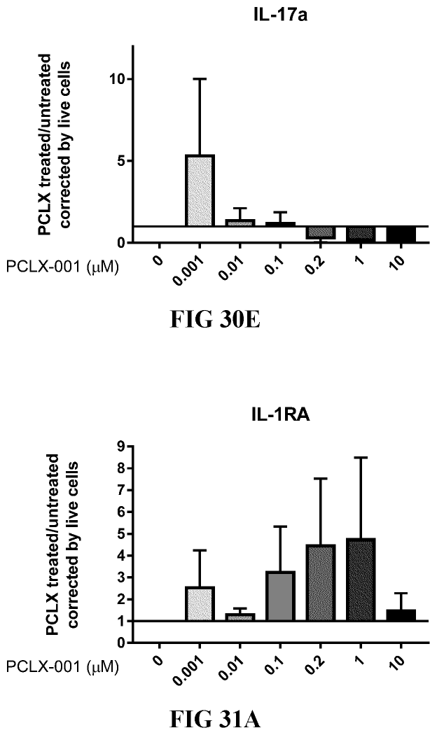

FIGS. 30 to 34 depict cytokine and chemokine production in cultured PBMC in presence of increasing amount of PCLX-001.

FIG. 35-36 depict pro and anti-inflamatory cytokine production in cultured T cells in presence of increasing amount of PCLX-001.

The following table lists chemokines and chemokine receptors.

| | | | |
|---|---|---|---|
| | Original | Chemokine | |
| Chemokine | Name | Receptor | Major Function |

CC Chemokines

| | | | |
|---|---|---|---|
| CCL1 | I-309 | CCR8 | Monocyte recruitment and endothelial |
| | | | cell migration |
| CCL2 | MCP-1 | CCR2 | Mixed leukocyte recruitment |
| CCL3 | MIP-1α | CCR1, CCR5 | Mixed leukocyte recruitment |
| CCL4 | MIP-1β | CCR5 | T cell, dendritic cell, monocyte, and |
| | | | NK recruitment; HIV coreceptor |
| CCL5 | RANTES | CCR1, CCR3, | Mixed leukocyte recruitment |
| | | CCR5 | |
| CCL7 | MCP-3 | CCR1, CCR2, | Mixed leukocyte recruitment |
| | | CCR3 | |
| CCL8 | MCP-2 | CCR3, CCR5 | Mixed leukocyte recruitment |
| CCL9 | MIP-1γ | CCR1 | DC recruitment, osteoclast |
| | | | differentiation |
| CCL11 | Eotaxin | CCR3 | Eosinophil, basophil, and TH2 |
| | | | recruitment |
| CCL 12 | MCP-5 | CCR2 | Mixed leukocyte recruitment |
| CCL13 | MCP-4 | CCR2, CCR3 | Mixed leukocyte recruitment |
| CCL 14 | HHC-1 | CCR1, CCR5 | |
| CCL 15 | MIP-18 | CCRI, CCR3 | Mixed leukocyte recruitment |
| CCL16 | HHC-4 | CCR1, CCR2 | Lymphocyte and monocyte recruitment |
| CCL17 | TARC | CCR4 | T cell recruitment |
| CCL18 | DC-CK1 | CCR8 | Lymphocyte and dendritic cell homing |
| CCL19 | MIP-3β/ELC | CCR7 | T cell and dendritic cell migration into |
| | | | parafollicular zones of lymph nodes |
| CCL20 | MIP-3α | CCR6 | Th17 recruitment, DC positioning in |
| | | | tissue |
| CCL21 | SLC | CCR7 | T cell and dendritic cell migration into |
| | | | parafollicular zones of lymph nodes |
| CCL22 | MDC | CCR4 | NK cell, T cell recruitment |
| CCL23 | MPIF-1 | CCR1 | Monocyte, neutrophil, T cell migration |
| CCL24 | Eotaxin-2 | CCR3 | Eosinophil, basophil, and T_{H}2 |
| | | | recruitment |
| CCL25 | TECK | CCR9 | Lymphocyte recruitment into intestine |
| CCL26 | Eotaxin-3 | CCR3 | Eosinophil, basophil, and T_{H}2 |
| | | | recruitment |
| CCL27 | CTACK | CCR10 | T cell recruitment into skin |
| CCL28 | MEC | CCR10 | T and B cell homing to mucosa |

CXC Chemokines

| | | | |
|---|---|---|---|
| CXCL1 | GROα | CXCR2 | Neutrophil recruitment |
| CXCL2 | GROβ | CXCR2 | Neutrophil recruitment |
| CXCL3 | GROγ | CXCR2 | Neutrophil recruitment |
| CXCL4 | PF4 | CXCR3B | Platelet aggregation |
| CXCL5 | ENA-78 | CXCR2 | Neutrophil recruitment |
| CXCL6 | GCP-2 | CXCR1, CXCR2 | Neutrophil recruitment |
| CXCL7 | NAP-2 | CXCR2 | Neutrophil recruitment |
| CXCL8 | IL-8 | CXCR1, CXCR-2 | Neutrophil recruitment |
| CXCL9 | Mig | CXCR3 | Effector T cell recruitment |
| CXCL10 | IP-10 | CXCR3, | Effector T cell recruitment |
| | | CXCR3B | |
| CXCL11 | I-TAC | CXCR3, CXCR7 | Effector T cell recruitment |
| CXCL12 | SDF-1αβ | CXCR4 | Mixed leukocyte recruitment; HIV |
| | | | coreceptor |
| CXCL13 | BCA-1 | CXCR5 | B cell migration into follicles; T |
| | | | follicular helper cell migration into |
| | | | follicles |
| CXCL 14 | BRAK | | Monocyte and dendritic cell migration |
| CXCL 16 | — | CXCR6 | Macrophage scavenger receptor |

C Chemokines

| | | | |
|---|---|---|---|
| XCL1 | Lymphotactin | XCR1 | T cell and NK cell recruitment |
| XCL2 | SCM-1β | XCL1 | |

CX₃C Chemokines

| | | | |
|---|---|---|---|
| CX₃CL1 | Fractalkine | CX₃CR1 | T cell, NK cell, and monocyte |
| | | | recruitment; CTL and NK cell activation |

FIGS. 35 and 36 show a deep reduction in pro inflammatory cytokine secretions from T cells in cells treated with -001 or -002. The effects are proportional to the potency of PCLX-001 vs PCLX-002 used. IMP-1088 also has significant inhibitory effects on cytokine secretion throughout other than for TNFa where it is increasing the secretion.

It is shown herein that that NMT inhibitors inhibit cytokine secretion and may be used as immunomodulator to reduce the activity of the T cells likely via the inhibition of the TCR with implication in auto-immune disease such as rhumatoid arthritis, Lupus, Sjogren's syndrome, type I diabetes, psoriasis, and in anti-inflammatory diseases (see lists below).

FIG. 35 NMT inhibitors (PCLX-001, PCLX-002, IMP-1088) reduce the normalized secretion of pro-inflammatory cytokines; IL-6 (A), IL-8 (B), TNF-α (C), and IFN-γ (D). T cells (n=3, from 3 independent donors) were incubated for 48 h with increasing concentration of NMT inhibitors, then induced by T cell activator (STEMCELLS Inc.) in the presence of the drugs for 2 more days. (Two-way ANOVA, P value against untreated: *<0.05-0.01 **<0.01-0.001 ***<0.001-0.0001. Reduction of cytokine secretion is stronger in the more potent NMT inhibitor PCLX-001 than PCLX-002 and that the survival of cells after 4 days of treatment was within 10% of untreated samples.

FIG. 36 NMT inhibitors (PCLX-001, PCLX-002, IMP-1088) reduce the normalized secretion of anti-inflammatory cytokines; IL-4 (A), IL-5 (B), IL-10 (C), and IL13 (D). T cells (n=3, from 3 independent donors) were incubated for 48 h with increasing concentration of NMT inhibitors, then induced by T cell activator (STEMCELLS) in the presence of the drugs for 2 more days. (Two-way ANOVA, P value against untreated: *<0.05-0.01 **<0.01-0.001 ***<0.001-0.0001. Reduction of cytokine secretion is stronger in the more potent NMT inhibitor PCLX-001 than PCLX-002 and that the survival of cells after 4 days of treatment was within 10% of untreated samples.

TABLE shoing T cell viability

| | | | |
|---|---|---|---|
| Drug Name | Sample # | Drug (nM) | Viabilty (%) |

T cells viability (#1)

| | | | |
|---|---|---|---|
| PCLX-001 | S17 | 0 | 9 |
| PCLX-001 | S18 | 20 | 9 |
| PCLX-001 | S19 | 100 | 8 |
| PCLX-001 | S20 | 500 | 10 |
| PCLX-002 | 522 | 0 | 10 |
| PCLX-002 | S23 | 20 | 9 |
| PCLX-002 | S24 | 100 | 9 |
| PCLX-002 | S25 | 500 | 9 |
| IMP-1088 | S27 | 0 | 8 |
| IMP-1088 | S28 | 20 | 9 |
| IMP-1088 | S29 | 100 | 10 |
| IMP-1088 | S30 | 500 | 6 |

T cells viability (#2)

| | | | |
|---|---|---|---|
| PCLX-001 | S47 | 0 | 77 |
| PCLX-001 | S48 | 20 | 66 |
| PCLX-001 | S49 | 100 | 60 |
| PCLX-001 | S50 | 500 | 62 |
| PCLX-002 | S52 | 0 | 67 |
| PCLX-002 | S53 | 20 | 65 |
| PCLX-002 | S54 | 100 | 65 |
| PCLX-002 | S55 | 500 | 65 |
| 1088 | S57 | 0 | 67 |
| 1088 | S58 | 20 | 61 |
| 1088 | S59 | 100 | 59 |
| 1088 | S60 | 500 | 59 |

T cells viability (#3)

| | | | |
|---|---|---|---|
| PCLX-001 | S77 | 0 | 79 |
| PCLX-001 | S78 | 20 | 76 |
| PCLX-001 | S79 | 100 | 71 |
| PCLX-001 | S80 | 500 | 68 |
| PCLX-002 | S82 | 0 | 75 |
| PCLX-002 | S83 | 20 | 75 |
| PCLX-002 | S84 | 100 | 75 |
| PCLX-002 | S85 | 500 | 69 |
| IMP-1088 | S87 | 0 | 74 |
| IMP-1088 | S88 | 20 | 74 |
| IMP-1088 | S89 | 100 | 70 |
| IMP-1088 | S90 | 500 | 70 |

### Materials and Method

### Jurkat T Cell Culture

Jurkat T cells were originally purchased from ATCC (https://www.atcc.org/products/tib-152). Cell lines were tested regularly for mycoplasma contamination using MycoAlert Plus Mycoplasma Detection Kit (Lonza, ME,USA). Jurkat T cells tested negative for mycoplasma contamination. Jurkat T cells were maintained in RPMI medium supplemented with 5% fetal bovine serum, 100 U/ml penicillin, 0.1 mg/ml streptomycin, 1 mM sodium pyruvate, and 2 mM L-glutamine.

### Primary Cell Culture

Primary human T αβT cells were derived from healthy donor blood as described (Siegers G M, Ribot E J, Keating A, Foster P J. Extensive expansion of primary human gamma delta T cells generates cytotoxic effector memory cells that can be labeled with Feraheme for cellular MRI. Cancer Immunol Immunother. (2013) 62:571-83. doi: 10.1007/s00262-012-1353-y). In brief, peripheral blood mononuclear cells were isolated and cultured in media containing 1 μg/ml Concanavalin A and 10 ng/ml IL-2 and IL-4. T cells expanded together for 6-8 days, and then conventional αβTc were depleted by magnetic cell separation. Viability and fold expansion were routinely assessed via Trypan Blue exclusion and cell counting. When fed, cells were diluted to one million cells/ml with complete medium (RPMI 1640 with 10% FBS, heat-inactivated, 1×MEM NEAA, 10 mM HEPES, 1 mM sodium pyruvate, 50 U/ml penicillin-streptomycin, and 2 mM L-glutamine-all from Invitrogen™, Thermo Fisher Scientific, Waltham, Massachusetts, USA) supplemented with 10 ng/ml IL-2 and IL-4 (Siegers G M, Dutta I, Kang E Y, Huang J, Kdbel M and Postovit L-M (2020) Aberrantly Expressed Embryonic Protein NODAL Alters Breast Cancer Cell Susceptibility to γδ T Cell Cytotoxicity. Front. Immunol. 11:1287. doi: 10.3389/fimmu.2020.01287).

The vial of primary mixed T cells for this experiments was thawed and five days post-thaw, cells were stained for flow cytometry, and then acquired seven days after thawing.

Incubation with Dasatinib and PCLX-001

Dasatinib was from Apex Bio Technology. PCLX-001 was identified as DDD86481 by Drs. David Gray and Paul Wyatt (University of Dundee, Scotland, UK) and provided by Pacylex Pharmaceuticals. Jurkat T cells were grown in six-well plates in 4 ml media/well and incubated with increasing concentrations of PCLX-001, dasatinib for up to 48 h.

### Activation of the T Cell Receptor

For TCR activation experiments, cells were incubated with 2 μg/ml a mix of human CD3 Monoclonal Antibody (OKT3), eBioscience™ and mouse CD28 Monoclonal Antibody (37.51), eBioscience™ (purchased from ThermoFisher Scientific) for up to 4 hours 2 min and the activation was stopped by the addition of 1 mM vanadate (Bio Basic Inc) solution in PBS.

### Immunoblotting

Rabbit anti-GAPDH (1:5000, affinity purified polyclonal, #EU1000,lot 1), was from laboratory stock and are available through Eusera (www.eusera.com). Rabbit monoclonal anti-Src (1:2000, clone 32G6, #2123, lot 5), Lck (1:2000, clone D88, #2984, lot 4 ERK (1:2000, clone 4695, #9102, lot 27), P-ERK (1:5000, clone 3510, #9101, lot 30),P-SFK (1:10,000, clone D49G4, #6943, lot 4) were purchased from Cell Signaling. echnologies. Rabbit monoclonal anti-BIP (1:2000, polyclonal, ADI-SPA-826) was purchased from Enzo Life Sciences. Mouse monoclonal anti-p-Tyr (1:10,000, PY99, sc-7020, lotI2118) antibody was purchased from Santa Cruz Biotechnology. Enhanced chemiluminescence (ECL) Prime Western blotting detection kits were purchased from GE Healthcare. Clarity ECL western blotting substrate was from Bio-Rad. Goat anti-human IgM (g chain) (70-8028-M002, lot S728028002001) was purchased from Tonbo biosciences.

Cells were harvested, washed in cold PBS, and lysed in 0.1% SDSRIPA buffer (50 mM Tris-HCl pH 8.0, 150 mM NaCl, 1% Igepal CA-630, 0.5% sodium deoxycholate, 2 mM MgCl2, 2 mM EDTA with 1× complete protease inhibitor; (Roche Diagnostics) by rocking for 15 min at 4° C. The lysates were centrifuged at 16,000 g for 10 min at 4° C., and the post-nuclear supernatant was collected.

Protein concentrations were determined by BCA assay (Thermo Scientific) according to manufacturer's instructions. Samples were prepared for electrophoresis by the addition of 5× loading buffer and boiled for 5 min. If not stated otherwise, 30 μg of total protein per lane is loaded on a 12.5% acrylamide gels. After electrophoresis, gels are transferred onto 0.2 M nitrocellulose membrane (Bio-Rad) thereafter probed with antibodies as described in materials section. Peroxidase activity is revealed following the procedure provided for the ECL Prime Western Blotting Detection Reagent (GE Healthcare, PA, USA).

### Human Cell Culture

Human peripheral blood mononuclear cells (PBMC) and purified T cells from healthy donors were purchased from STEMCELL Technologies (CA). Cells (7.5×10⁶/ml) were cultured in the RPMI supplementing with 10% heat-inactivated fetal bovine serum (vWR), 1% penicillin/streptomycin (SigmaMillipore), 1% sodium pyruvate, and 1% non-essential amino acids (Gibco) in 24 well plates in the presence of 100 IU/ml interleukin 2 (STEMCELL Technologies or HIV reagent program (managed by ATCC).

For PBMC samples, cells were treated with various concentrations of PCLX-001 (0-10 ug/ml) for 2 and/or 4 days. Harvested cells were first stained for viability using Zombie aqua, blocked FC receptors using human Trustain FcX Fc receptor blocking solution, and labeled with fluorophore conjugated monoclonal antibodies (CD3, CD4, CD8, CD19, and CD14) all from Biolegend, respectively. Then, cells were permeabilized with fix/perm buffer kit (eBiosciences) and intracellularly stained with anti-Lyn and HGAL antibodies. The samples were acquired using LSRFortessa X20 (BD Biosciences) and analyzed by FlowJo software.

For purified T cells, the PCLX-001, PCLX-002 (PACYLEX), and IMP-1088(?) were added in various concentrations (0-500 nM) for 2 days. Then, cells were induced with T cell activator (STEMCELLS) in the presence of the drugs for 2 more days. After 4 days, the viability of treated T cells was analyzed using Flow cytometry. The supernatants for both PBMC and T cells were collected for further analysis.

### Multiplex Array Assay

The collected supernatants were analyzed for various biomarkers using multiplex cytokine array (Eve Technologies Discovery assay, Calgary, CA) either human cytokine/chemokine 71-Plex (HD71) for PBMC or human proinflammatory focused 15-Plex (HDF15) for T cells samples.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1-44. (canceled)

45. A method of treating an autoimmune disorder in a subject, comprising: administering a therapeutically effective amount of PCLX-001, a therapeutically effective amount of DDD85646, a therapeutically effective amount of IMP 1008, or a therapeutically effective amount of an NMT inhibitor, wherein the subject is a human.

46-48. (canceled)

49. The method of claim 45, wherein said autoimmune disorder is rheumatoid arthritis, asthma, multiple sclerosis, myasthenia gravis, lupus erythematosus, insulin-dependent diabetes (type 1), gastritis, colitis, and insulin-dependent autoimmune diabetes, graft transplant/inhibition of rejection, psoriasis, Sjogren's syndrome or graft vs host disease.

50. (canceled)

51. A method of treating an inflammatory disorder in a subject, comprising: administering a therapeutically effective amount of PCLX-001, a therapeutically effective amount of DDD85646, a therapeutically effective amount of IMP 1008, or a therapeutically effective amount of an NMT inhibitor, wherein the subject is a human.

52-54. (canceled)

55. The method of claim 51, wherein said inflammatory disorder is acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, ulcerative inflammation, a gastrointestinal disorder, a peptic ulcer, a regional enteritis, diverticulitis, gastrointestinal bleeding, eosinophilic, eosinophilic esophagitis, eosinophilic gastritis, eosinophilic gastroenteritis, eosinophilic colitis, gastritis, diarrhea, gastroesophageal reflux disease (GORD, or GERD), inflammatory bowel disease (IBD), Crohn's disease, ulcerative colitis, collagenous colitis, lymphocytic colitis, ischaemic colitis, diversion colitis, Behcet's syndrome, indeterminate colitis, or inflammatory bowel syndrome (IBS), or a disorder of the lung selected from the group consisting of pleurisy, alveolitis, vasculitis, pneumonia, chronic bronchitis, bronchiectasis, diffuse panbronchiolitis, hypersensitivity pneumonitis, asthma, idiopathic pulmonary fibrosis (IPF), and cystic fibrosis.

56. (canceled)

57. A method of reducing a BCR protein level or activity and/or TCR protein level or activity in a cell of a subject, comprising: contacting said cell with PCLX-001, DDD85646, or an NMT inhibitor, wherein said subject is a human.

58-60. (canceled)

61. The method of claim 57, wherein said contacting is in vitro or in vivo.

62. The method of claim 57, comprising a plurality of said cells.

63. A method reducing the activity of an immune cell from a subject or of reducing the activity of a T-cell and/or a B-cell from a subject, comprising: contacting said T-cell and/or said B-cell with an NMT inhibitor, wherein the subject is a human.

64. (canceled)

65. The method of claim 63, wherein said NMT inhibitor is PCLX-001, DDD85646, or IMP 1088.

66-68. (canceled)

69. The method of claim 63, wherein said contacting is in vitro or in vivo.

70-94. (canceled)

95. A method reducing the activity of a monocyte cell in a subject or reducing the number of monocyte cells in a subject, comprising: contacting said monocyte with an NMT inhibitor.

96. The method of claim 95, wherein said NMT inhibitor is PCLX-001, DDD85646, or IMP 1088, and wherein the subject is a human.

97-99. (canceled)

100. The method of claim 95, wherein said contacting is in vitro or in vivo.

101-106. (canceled)

107. A method of reducing the amount of cytokine secretion in a T-cell in a subject, comprising: administering an NMT inhibitor, wherein said cytokine is IL-6, IL-8, IFN-gamma, IL-5, IL-10, or IL-13, and wherein the subject is a human.

108. (canceled)

109. The method of claim 107, wherein said NMT inhibitor is PCLX-001, DDD85646, or IMP-1088.

110-112. (canceled)

113. The method of claim 107, wherein said contacting is in vitro or in vivo.

114-120. (canceled)
